(19) United States
(12) Patent Application Publication
(10) Pub. No.: US 2010/0035866 A1
(43) Pub. Date: Feb. 11, 2010

(54) NOVEL SUBSTITUTED-1, 1-DIOXO-BENZO[1,2,4]THIADIAZIN-3ONES, PREPARATION METHOD THEREOF, AND PHARMACEUTICAL COMPOSITION CONTAINING THE SAME

(75) Inventors: Churlmin Seong, Daejeon (KR); Jinil Choi, Daejeon (KR); Chul Min Park, Daejeon (KR); Wookyu Park, Chungcheongbuk-do (KR); Jaeyang Kong, Daejeon (KR); Sunhee Kang, Daejeon (KR); Chimin Park, Daejeon (KR)

Correspondence Address: LUCAS & MERCANTI, LLP, 475 PARK AVENUE SOUTH, 15TH FLOOR, NEW YORK, NY, 10016, US

(73) Assignee: KOREA RESEARCH INSTITUTE OF CHEMICAL TECHNOLOGY, Daejeon (KR)

(21) Appl. No.: 12/293,965

(22) PCT Filed: Mar. 28, 2006
(86) PCT No.: PCT/KR2006/001127

§ 371 Date: Sep. 22, 2008

(30) Foreign Application Priority Data
Mar. 23, 2006 (KR) 10-2006-0026492

(51) Int. Cl. A61K 31/5415; C07D 285/24; A61P 25/28; A61P 25/30; A61P 25/24; A61P 25/22; A61P 25/32

(52) U.S. Cl. 514/223.2; 544/12

(57) ABSTRACT

The present invention relates to compounds of substituted-1,1-dioxo-benzo[1,2,4]thiadiazin-3-ones acting as a 5HT6 receptor antagonist, a preparation method thereof, and a pharmaceutical composition containing the same for treatment of the central nervous system disorders. The compounds of substituted-1,1-dioxo-benzo[1,2,4]thiadiazin-3-ones according to the present invention have excellent binding affinity for the 5HT6 receptor and excellent selectivity for the 5HT6 receptor over other receptors. Also, the compounds reverse a disruption of PPI by apomorphine and don't show rotatod deficit in mice. Therefore the compounds according to the present invention may be valuably used for treatment of a 5HT6 receptor relating disorders.

### TECHNICAL FIELD

The present invention relates to substituted-1,1-dioxo-benzo[1,2,4]thiadiazin-3-ones acting as a 5-HT6 receptor antagonist, a preparation method thereof, and a pharmaceutical composition containing the same for treatment of central nervous system disorders.

### BACKGROUND ART

Although the function of serotonin (5-HT) in the central nervous system is still being clarified, various studies have indicated 5-HT has been implicated in the aetiology of many disease states and may be particularly important in mental illness, such as depression, anxiety, schizophrenia, eating disorders, obsessive compulsive disorder (OCD), migraine and panic disorder. Recent advances in pharmacology, molecular biology, and genetics on the serotonin system hold out the promise of the development of improved pharmacological treatment for some aspects of neurological diseases. Indeed, many currently used treatments of these disorders are thought to act by modulating serotonergic neurons. During the last decade, multiple 5-HT receptor subtypes have been characterized. Initially, receptor subtypes were characterized using pharmacological tools only. On the basis of the receptor binding profiles, common secondary messenger coupling and the functional activity of ligands, four main subgroups of 5-HT receptors, termed 5-HT1, 5-HT2, 5-HT3 and 5-HT4, were identified. More recently, molecular biological techniques have both confirmed this classification, in that each subgroup has been found to have relatively dissimilar protein structures, and led to the identification of novel 5-HT receptors (5-HT1F, 5-HT5, 5-HT6 and 5-HT7) enabling them to be cloned, expressed in cultured cell lines [Hoyer, D. et al., Pharmacol. Biochem. Behav., 2002, 71, 533-554; Kroeze, W. K. et al., Curr. Top. Med. Chem., 2002, 2, 507-528].

Most recently, the 5-HT6 receptor has been cloned from rat cDNA based on its homology to previously cloned G-protein-coupled receptors. The rat receptor consists of 438 amino acids with seven transmembrane domains and is positively coupled to adenylyl cyclase via the Gs G-protein [Monsma, F. J. et al., Mol. Pharmacol., 1993, 43, 320-327]. Human 5-HT6 receptors, a 440 amino acid polypeptide, display 89% overall sequence homology with the rat receptors and is positively coupled to an adenylate cyclase second messenger system [Kohen, R. et al., J. Neurochem., 1996, 66, 47-56]. Rat and human 5-HT6 mRNA is located in the striatum, amygdala, nucleus accumbens, hippocampus, cortex and olfactory tubercle, but has not been found in peripheral organs studied. In pharmacological studies, tritiated 5-HT, tritiated LSD, and [125I]-2-iodo LSD have been used to radiolabel 5-HT6 receptors. 5-HT binds with moderately high affinity (Ki=50-150 nM). Tricyclic antipsychotic agents and some antidepressants bind with significant affinity. A related investigation examined antipsychotics in greater detail and found that representative members of several classes of antipsychotics bind with high affinity. Examples include phenothiazine chlorpromazine, thioxanthene chlorprothixene, diphenylbutylpiperidine pimozide, heterocyclic antipsychotic agent loxapine and clozapine [Roth, B. L. et al., J. Pharmacol. Exp. Ther., 1994, 268, 1403-1410]. These results led to suggestions that 5-HT6 receptors might play a role in certain types of psychoses and that they might represent significant targets for the atypical antipsychotics in particular.

Until selective ligands were developed, exploration of 5-HT6 pharmacology was largely dependent on the use of nonselective agents. In the absence of selective ligands for the receptor, functional studies have been carried out using an antisense approach. 5-HT6 specific antisense produced a specific behavioural syndrome of yawning, stretching and chewing, but had no other discernable action in rats. The non-selective ligands were useful for investigating the pharmacology of 5-HT6 systems in preparations where other 5-HT receptors were absent (e.g., cAMP assays); however, owing to their lack of selectivity, they were of limited value for most other pharmacological studies.

Recent advent of selective agents has greatly benefited 5-HT6 studies, and this field of research has recently exploded. The development of more selective ligands may therefore lead to treatments with increased efficacy and reduced side effects. Alternatively, selective ligands may form completely novel therapies. It was not until 1998 that the first 5-HT6-selective antagonist was described, and this prompted others to quickly report their efforts in this area. Sleight et al. at Hoffman-La Roche Co. identified the bisaryl sulfonamides Ro 04-6790 (1, Ki=55 nM), Ro 63-0563 (2, Ki=12 nM) as very selective 5-HT6 antagonists [Sleight, A. J. et al., Br. J. Pharmacol., 1998, 124, 556-562]. Shortly thereafter, MS-245 (3, Ki=2.3 nM) was reported. Interestingly, although they represented independent discoveries, all three were identified by random screening methods and all three possess a sulfonamide moiety.

One problem associated with these antagonists was their low penetration of the CNS. At the time, Smith-Kline Beecham Co. also pinched out compound 4 via high-throughput screening. It displayed high affinity (Ki=5 nM) for 5-HT6 receptors and >50-fold selectivity over 10 other 5-HT receptors and no measurable affinity for 50 other receptor/binding sites. It was a pure antagonist of cAMP accumulation (pKb=7.8) [Bromidge, S. M. et al., J. Med. Chem., 1999, 42, 202-205]. It was moderately brain penetrant (25%) but subject to rapid blood clearance resulting in low bioavailability.

An ensuing structure activity study identified SB-271046 (5, Ki=1 nM; >200 selectivity over 50 other receptors) retained antagonist activity, and although less brain-penetrant (10%), it showed excellent (>80%) oral bioavailability.

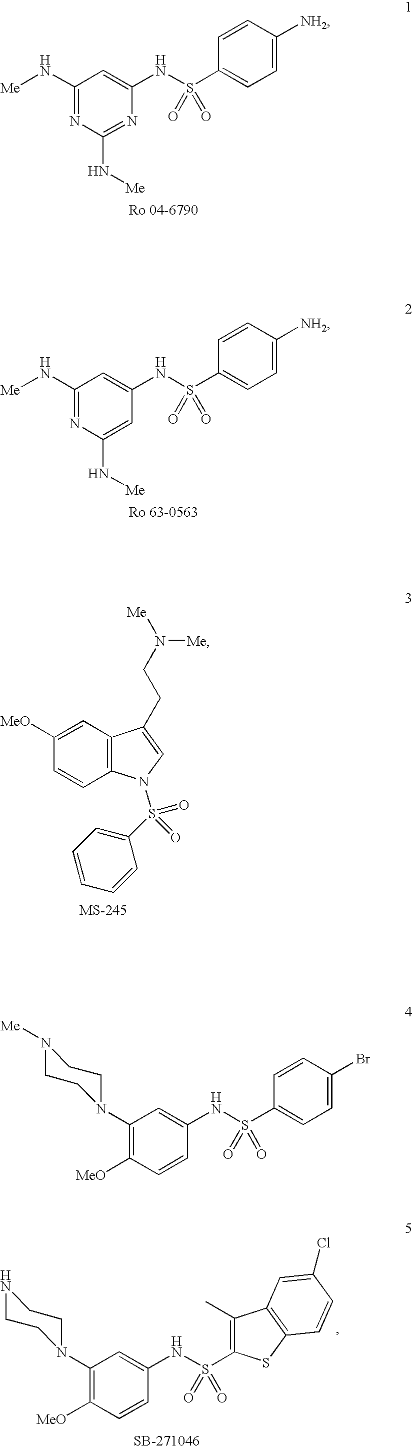

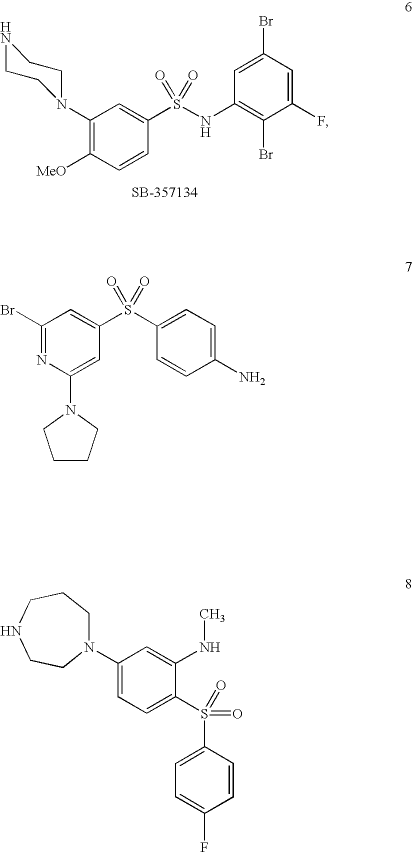

Subsequent studies by this group showed that SB-357134 (6, Ki=3 nM) with a low clearance rate and excellent oral bioavailability. In 1999, Glennon et al. undertook a structure affinity investigation of the binding of tryptamine derivatives at human 5-HT6 receptors [Glennon, R. A. et al., J. Med. Chem., 2000, 43, 1011-1018]. MS-245 was found as an antagonist (pA2=8.88) with high affinity (Ki=2.3 nM). In contrast to the above-mentioned sulfonamides or tryptamine derivatives, Hoffmann-LaRoche (7) and Pharmacia-Upjohn (8, Ki=1.4 nM) recently revealed several sulfones [Slassi, A. et al., Expert Opin. Ther. Pat., 2002, 12, 513-527]. Newer agents continue to be developed in attempts to improve pharmacokinetic and pharmacodynamic properties. Now that some tools are available, attention is focusing more and more on the function of 5-HT6 receptors.

Atypical antipsychotics, in particular, display high affinity at these receptors (vide supra). In addition, the tritiated atypical antipsychotic agent [3H]clozapine was shown to label two populations of receptors in rat brain and one population was thought to represent 5-HT6 receptors [Glatt, C. E. et al., Mol. Med., 1995, 1, 398-406]. Vogt et al. performed a systematic mutation scan of the coding region of the 5-HT6 receptor gene of 137 individuals (including schizophrenic and depressed patients) and concluded that the gene might be involved in bipolar affective disorder [Vogt, I. R. et al., Am. J. Med. Genet., 2000, 96, 217-221].

Prior to the identification of 5-HT6-selective agents, Bourson et al. demonstrated that intracerebroventricular administration of antisense oligonuceotides produced in rats a specific behavior of yawning, stretching, and chewing, which could be antagonized by atropine [Bourson, A. et al., J. Pharmacol. Exp. Ther., 1995, 274, 173-180]. Sleight et al. demonstrated that Ro 04-6790 (1) was capable of inducing this same effect. Owing to a relationship between cholinergic function and cognition, this led to speculation that 5-HT6 receptors might be involved in memory and cognitive dysfunction [Sleight, A. J. et al., Neuropharmacology, 2001, 41, 210-219; Rogers, D. C. et al., Psychopharmacology (Berlin), 2001, 158, 114-119].

In addition, because antisense oligonucleotide pretreatment and Ro 04-6790 administration both led to decreased food intake by rats, it was suggested that 5-HT6 receptors might be involved in the regulation of feeding. Furthermore, Russell and Dias have questioned the postulate that 5-HT6 antagonists increase cholinergic transmission [Russell, M. G. N.; Dias, R., Curr. Top. Med. Chem., 2002, 2, 643-654].

Despite the mechanistic disagreement, there is evidence for the involvement of 5-HT6 receptors in learning and memory. When a water maze was used with rats as subjects, SB-271046 (5) and SB-357134 (6) showed significant improvement in retention of a previously learned task. Furthermore, SB-271046 (5) increased extracellular glutamate levels in frontal cortex and dorsal hippocampus by several fold, leading to the conclusion that selective enhancement of excitatory neurotransmission by SB-271046 supports a role for 5-HT6 receptor antagonists in the treatment of cognitive disorders and memory dysfunction [Dawson, L. A. et al., Neuropsychopharmacology, 2001, 25, 662-668].

In addition, SB-357134 (6) produced a potent and dose-dependent increase in seizure threshold (rat maximal electroseizure threshold) following oral administration, suggesting possible therapeutic utility in convulsive disorders [Stean, T. O. et al., Pharmacol. Biochem. Behav., 2002, 71, 645-654]. These findings are consistent with an earlier finding that SB-271046 (5) and Ro 04-6790 (1) possess anticonvulsant activity.

Overall, there is some evidence to suggest that 5-HT6 receptors could be involved in psychosis. There is still more evidence that these receptors are involved in cognition and learning and additional evidence that they might have a role in convulsive disorders and appetite control. Although additional studies are certainly warranted, particularly with some of the newer 5-HT6 antagonists that are more brain-penetrant than the earlier agents, the future of 5-HT6 receptor ligands as potential therapeutic agents is quite exciting.

The inventors made an effort to develop a 5-HT6 antagonist having excellent binding affinity and selectivity, and has completed the present invention by discovering that benzothiadiazine derivatives are 5-HT6 antagonists having very excellent binding strength and selectivity compared to sulfonamide or sulfonic structures disclosed in the prior art.

### DISCLOSURE OF INVENTION

### Technical Problem

The present invention provides substituted-1,1-dioxo-benzo[1,2,4]thiadiazin-3-ones and a pharmaceutically acceptable salt thereof.

Additionally, the present invention provides a preparation method for substituted-1,1-dioxo-benzo[1,2,4]thiadiazin-3-ones.

Additionally, the present invention provides a pharmaceutical composition including substituted-1,1-dioxo-benzo[1,2,4]thiadiazin-3-ones, a pharmaceutically acceptable salt thereof or prodrug thereof for treatment of the central nervous system disorders.

### ADVANTAGEOUS EFFECTS

The compounds of substituted-1,1-dioxo-benzo[1,2,4]thiadiazin-3-ones according to the present invention have excellent binding affinity to the 5-HT6 receptor, excellent selectivity to the 5-HT6 receptor over other receptors, inhibition of the serotonin (5-HT)-stimulated cAMP accumulation and an effect on apomorphine (2 □/□, i.p.)-induced disruption of prepulse inhibition (PPI) in rats. Also, the compounds of the present invention of effective dose don't show any rotarod deficits in mice.

### BRIEF DESCRIPTION OF THE DRAWINGS

FIG. 1 is a graph showing an inhibitory effect of compounds according to the example (Example 44) of the present invention and methiothepin on cAMP accumulation mediated by 5-HT6 receptor of human HeLa cell.

FIG. 2 is a graph showing an inhibitory effect of compounds according to the example (Example 1, Example 44) of the present invention (50 □/□, i.p.) on hyperactivity of a rat induced by apomorphine (2□/□, i.p.).

### BEST MODE FOR CARRYING OUT THE INVENTION

The present invention provides substituted-1,1-dioxo-benzo[1,2,4]thiadiazin-3-ones represented by Formula 1, a pharmaceutically acceptable salt and prodrug thereof.

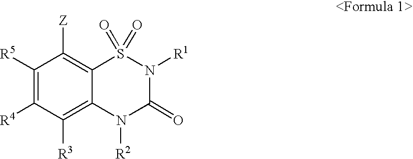

wherein,

R¹ represents hydrogen, C₁˜C₁₀ alkyl, C₃˜C₁₀ aryl, C₃˜C₇ cycloalkyl, arylalkyl, heteroaryl or heteroarylalkyl,

R² represents hydrogen, C₁˜C₁₀ alkyl, C₃˜C₁₀ aryl, heteroaryl, aralkyl, heteroarylalkyl, amino or cyclic amino,

R³, R⁴ and R⁵ independently represent hydrogen, halogen, amino, cyclic amino, nitro, cyano, C₁˜C₁₀ alkyl, haloalkyl, C₁˜C₇ alkoxy, haloalkoxy or piperazinyl or N-methyl piperazinyl, and

Z represents saturated mono-, bi-, tricyclic amines containing 1 to 3 nitrogen atoms and 5 to 12 carbon atoms in the ring.

Term “alkyl” as used herein means straight and branched chain containing from 1 to 10 carbon atoms and includes methyl, ethyl, propyl, isopropyl, butyl, sec-butyl, and tert-butyl, pentyl, hexyl, octyl, decyl, cyclopropylmethyl, cyclohexylmethyl and the like.

The term “cycloalkyl” refers to carbocyclic ring containing from 3 to 7 carbon atoms, includes cyclopropyl, cyclobutyl, cyclopentyl cyclohexyl, cycloheptyl.

Term “alkoxy” as used herein means straight and branched alkoxy groups containing from 1 to 7 carbon atoms, includes methoxy, ethoxy, propyloxy, iso-propyloxy, butoxy, sec-butoxy, and tert-butoxy, pentoxy, hexyloxy, cyclo-hexylmethoxy and the like.

Term “haloalkyl” means alkyl groups substituted by one or more fluorine, chlorine atoms, e.g. fluoromethyl, difluoromethyl, trifluoromethyl, pentafluoroethyl, 1,1-difluoroethyl and trichloromethyl.

The term “aryl” refers to carbocylic aromatic group, include phenyl, naphthyl, phenanthryl, anthracyl, indenyl, biphenyl and fluorenyl etc.

The term “heteroaryl” refers to an aryl group containing from 1 to 3 selected from O, N and S, and includes pyridyl, quinolinyl, isoquinolinyl, pyridazinyl, pyrimidinyl, pyrazinyl, pyrrolyl, indolyl, pyranyl, furyl, benzimidazolyl, benzofuryl, thienyl, benzthienyl, imidazolyl, oxadiazolyl, thiazolyl and thiadiazolyl.

The aryl and heteroaryl groups are optionally substituted by 1, 2 or 3 independently selected substituents which include halogen, nitro, amino, cyano, cyclic amino, hydroxy, carboxylic acid, thiol, alkyl, aryl, heteroalkyl, heteroaryl, alkoxy, aryloxy, acyloxy, acylamino, arylsulfonylamino, arylsulfonylureido, heteroaryl, alkylthio, arylthio, alkylcarboxylate, arylcarboxylate, aralkylcarboxylate, alkylureido, arylureido, alkylamindino or arylamidino etc.

The term “heteroarylalkyl” refers to C₁˜C₄ alkyl groups containing above-mentioned heteroaryl groups. As the same way, the term “arylalkyl” refers to alkyl groups containing above-mentioned aryl groups.

The term “amino” include NH, NHR⁷ and NR⁷R⁸, wherein R⁷ and R⁸ are C₁˜C₄ alkyl groups. The term “cyclic amino” include piperidinyl, piperazinyl and morpholinyl groups.

Typical halogen atoms include fluorine, chlorine, bromine and iodine.

Preferably,

R¹ represents C₁˜C₁₀ alkyl, C₃˜C₇ cycloalkyl; phenyl, benzyl, naphtalenyl, pyridinyl, furanyl; C₃˜C₇ cycloalkyl, phenyl, benzyl, naphtalenyl, pyridinyl or furanyl substituted by 1 or 2 substituent selected from a group comprising of C₁˜C₄ alkyl, C₁ ˜C₄ alkoxy, halogen, nitro, amino, cyano, hydroxy and methyl carboxylate,

R² represents C₁˜C₄ alkyl, phenyl or benzyl,

R³, R⁴ and R⁵ independently represent hydrogen, halogen or methoxy, and

Z presents piperazinyl, piperazinyl substituted by C₁˜C₄ alkyl or amine, morpholinyl, pyrrolyl, pyridinyl or diazabicycloalkyl.

More preferably,

R¹ presents methyl, ethyl, propyl, n-butyl, octyl, decyl; phenyl, benzyl, furanyl; cyclohexylmethyl, phenethyl, (R)-1-phenyl-ethyl, (S)-1-phenyl-ethyl, phenylpropyl, methoxyphenyl, dimethylphenylpropyl, fluorobenzyl, chlorobenzyl, bromobenzyl, methylbenzyl, methoxybenzyl, iodobenzyl, hydroxybenzyl, nitrobenzyl, cyanobenzyl, methyl carboxylatebenzyl, naphtalenylmethyl or pyridinylmethyl,

R² presents benzyl,

R³, R⁴ and R⁵ independently present hydrogen, chlorine, bromine or methoxy, and

Z presents piperazinyl, methylpiperazinyl, pyridinyl-piperazinyl, morpholinyl, diazabicyclononyl, diazabicyclodecyl or diazabicyclooctyl.

Salts of the compounds of Formula 1 according to the present invention should be a pharmaceutically accepted non-toxic salt in order to be used as a medicine, and other salts may, however, be useful in the preparation of the compounds according to the invention or of their non-toxic pharmaceutically acceptable salts.

The pharmaceutically acceptable salts include alkali metal salts such as lithium, sodium or potassium salts; alkaline earth metal such as calcium or magnesium salts; and salts formed with suitable organic ligands such as quaternary ammonium salts. In the case of acid addition salt, for example, a solution of the compound according to the present invention may be mixed with pharmaceutically acceptable non-toxic acid solution such as hydrochloric acid, fumaric acid, maleic acid, succinic acid, acetic acid, citric acid, tartaric acid, carbonic acid or phosphoric acid.

The compounds according to the present invention include prodrugs of the compounds of Formula 1. Generally, such prodrugs will be functional derivatives of the compounds of Formula 1 which are readily converted in vivo into the required compounds. The suitable prodrugs according to the present invention may be selected and prepared by a conventional method [“Design of Prodrugs”, ed. H. Bundgaard, Elsevier, 1985].

The compounds according to the present invention include various tautomers of the compounds of Formula 1.

Where the compounds according to the invention have at least one asymmetric center, they may accordingly exist as enantiomers. Where the compounds according to the invention possess two or more asymmetric centres, they may additionally exist as diastereoisomers. It is to be understood that all such isomers and mixtures thereof are encompassed within the scope of the present invention.

More preferably, the compounds of Formula 1 according to the present invention, a pharmaceutically acceptable salts and prodrug thereof are selected from the group consisting of:
(1) 2,4-Dibenzyl-6-chloro-8-(4-methyl-piperazin-1-yl)-1,1-dioxo-1,4-dihydro-2H-1λ⁶-benzo[1,2,4]thiadiazin-3-one,
(2) 4-Benzyl-6-chloro-2-(2-fluoro-benzyl)-8-(4-methyl-piperazin-1-yl)-1,1-dioxo-1,4-dihy dro-2H-1λ⁶-benzo[1,2,4]thiadiazin-3-one,
(3) 4-Benzyl-6-chloro-2-(3-fluoro-benzyl)-8-(4-methyl-piperazin-1-yl)-1,1-dioxo-1,4-dihy dro-2H-1λ⁶-benzo[1,2,4]thiadiazin-3-one,
(4) 4-Benzyl-6-chloro-2-(4-fluoro-benzyl)-8-(4-methyl-piperazin-1-yl)-1,1-dioxo-1,4-dihy dro-2H-1λ⁶-benzo[1,2,4]thiadiazin-3-one,
(5) 4-Benzyl-6-chloro-2-(2-chloro-benzyl)-8-(4-methyl-piperazin-1-yl)-1,1-dioxo-1,4-dihy dro-2H-1λ⁶-benzo[1,2,4]thiadiazin-3-one,
(6) 4-Benzyl-6-chloro-2-(3-chloro-benzyl)-8-(4-methyl-piperazin-1-yl)-1,1-dioxo-1,4-dihy dro-2H-1λ⁶-benzo[1,2,4]thiadiazin-3-one,
(7) 4-Benzyl-6-chloro-2-(4-chloro-benzyl)-8-(4-methyl-piperazin-1-yl)-1,1-dioxo-1,4-dihy dro-2H-1λ⁶-benzo[1,2,4]thiadiazin-3-one,
(8) 4-Benzyl-2-(2-bromo-benzyl)-6-chloro-8-(4-methyl-piperazin-1-yl)-1,1-dioxo-1,4-dihy dro-2H-1λ⁶-benzo[1,2,4]thiadiazin-3-one,
(9) 4-Benzyl-2-(3-bromo-benzyl)-6-chloro-8-(4-methyl-piperazin-1-yl)-1,1-dioxo-1,4-dihy dro-2H-1λ⁶-benzo[1,2,4]thiadiazin-3-one,
(10) 4-Benzyl-2-(4-bromo-benzyl)-6-chloro-8-(4-methyl-piperazin-1-yl)-1,1-dioxo-1,4-dihy dro-2H-1λ⁶-benzo[1,2,4]thiadiazin-3-one,
(11) 4-Benzyl-6-chloro-2-(3-iodo-benzyl)-8-(4-methyl-piperazin-1-yl)-1,1-dioxo-1,4-dihydro-2H-1λ⁶-benzo[1,2,4]thiadiazin-3-one,
(12) 4-Benzyl-6-chloro-2-(4-iodo-benzyl)-8-(4-methyl-piperazin-1-yl)-1,1-dioxo-1,4-dihydro-2H-1λ⁶-benzo[1,2,4]thiadiazin-3-one,
(13) 4-Benzyl-6-chloro-2-(2-methyl-benzyl)-8-(4-methyl-piperazin-1-yl)-1,1-dioxo-1,4-dihydro-2H-1λ⁶-benzo[1,2,4]thiadiazin-3-one,
(14) 4-Benzyl-6-chloro-2-(3-methyl-benzyl)-8-(4-methyl-piperazin-1-yl)-1,1-dioxo-1,4-dihydro-2H-1λ⁶-benzo[1,2,4]thiadiazin-3-one,
(15) 4-Benzyl-6-chloro-2-(4-methyl-benzyl)-8-(4-methyl-piperazin-1-yl)-1,1-dioxo-1,4-dihydro-2H-1λ⁶-benzo[1,2,4]thiadiazin-3-one,
(16) 4-Benzyl-6-chloro-2-(2-methoxy-benzyl)-8-(4-methyl-piperazin-1-yl)-1,1-dioxo-1,4-dihydro-2H-1λ⁶-benzo[1,2,4]thiadiazin-3-one,
(17) 4-Benzyl-6-chloro-2-(3-methoxy-benzyl)-8-(4-methyl-piperazin-1-yl)-1,1-dioxo-1,4-dihydro-2H-1λ⁶-benzo[1,2,4]thiadiazin-3-one,
(18) 4-Benzyl-6-chloro-2-(4-methoxy-benzyl)-8-(4-methyl-piperazin-1-yl)-1,1-dioxo-1,4-dihydro-2H-1λ⁶-benzo[1,2,4]thiadiazin-3-one,
(19) 4-Benzyl-8-chloro-2-(3-hydroxy-benzyl)-6-(4-methyl-piperazin-1-yl)-1,1-dioxo-1,4-dihydro-2H-1λ⁶-benzo[1,2,4]thiadiazin-3-one,
(20) 4-Benzyl-6-chloro-8-(4-methyl-piperazin-1-yl)-2-(2-nitro-benzyl)-1,1-dioxo-1,4-dihydro-2H-1λ⁶-benzo[1,2,4]thiadiazin-3-one,
(21) 4-Benzyl-6-chloro-8-(4-methyl-piperazin-1-yl)-2-(3-nitro-benzyl)-1,1-dioxo-1,4-dihydro-2H-1λ⁶-benzo[1,2,4]thiadiazin-3-one,
(22) 4-Benzyl-6-chloro-8-(4-methyl-piperazin-1-yl)-2-(4-nitro-benzyl)-1,1-dioxo-1,4-dihydro-2H-1λ⁶-benzo[1,2,4]thiadiazin-3-one,
(23) 4-[4-Benzyl-6-chloro-8-(4-methyl-piperazin-1-yl)-1,1,3-trioxo-3,4-dihydro-1H-1λ⁶-benzo[1,2,4]thiadiazin-2-ylmethyl]-benzoic acid methyl ester,
(24) 4-[4-Benzyl-6-chloro-8-(4-methyl-piperazin-1-yl)-1,1,3-trioxo-3,4-dihydro-2H-1λ⁶-benzo[1,2,4]thiadiazin-2-ylmethyl]-benzonitrile,
(25) 4-Benzyl-6-chloro-8-(4-methyl-piperazin-1-yl)-1,1-dioxo-2-[(R)-1-phenyl-ethyl]-1,4-dihydro-2H-1λ⁶-benzo[1,2,4]thiadiazin-3-one,
(26) 4-Benzyl-6-chloro-8-(4-methyl-piperazin-1-yl)-1,1-dioxo-2-[(S)-1-phenyl-ethyl]-1,4-dihydro-2H-1λ⁶-benzo[1,2,4]thiadiazin-3-one,
(27) 4-Benzyl-6-chloro-8-(4-methyl-piperazin-1-yl)-1,1-dioxo-2-phenyl-1,4-dihydro-2H-1λ⁶-benzo[1,2,4]thiadiazin-3-one,
(28) 4-Benzyl-6-chloro-2-(2-methoxy-phenyl)-8-(4-methyl-piperazin-1-yl)-1,1-dioxo-1,4-dihydro-2H-1λ⁶-benzo[1,2,4]thiadiazin-3-one,
(29) 4-Benzyl-6-chloro-2-(3-methoxy-phenyl)-8-(4-methyl-piperazin-1-yl)-1,1-dioxo-1,4-dihydro-2H-1λ⁶-benzo[1,2,4]thiadiazin-3-one,
(30) 4-Benzyl-6-chloro-2-(4-methoxy-phenyl)-8-(4-methyl-piperazin-1-yl)-1,1-dioxo-1,4-dihydro-2H-1λ⁶-benzo[1,2,4]thiadiazin-3-one,
(31) 6-Chloro-2,4-dimethyl-8-(4-methyl-piperazin-1-yl)-1,1-dioxo-1,4-dihydro-2H-1λ⁶-benzo[1,2,4]thiadiazin-3-one,
(32) 4-Benzyl-6-chloro-2-methyl-8-(4-methyl-piperazin-1-yl)-1,1-dioxo-1,4-dihydro-2H-1λ⁶-benzo[1,2,4]thiadiazin-3-one,
(33) 4-Benzyl-6-chloro-2-ethyl-8-(4-methyl-piperazin-1-yl)-1,1-dioxo-1,4-dihydro-2H-1λ⁶-benzo[1,2,4]thiadiazin-3-one,
(34) 4-Benzyl-6-chloro-8-(4-methyl-piperazin-1-yl)-2-propyl-1,1-dioxo-1,4-dihydro-2H-1λ⁶-benzo[1,2,4]thiadiazin-3-one,
(35) 4-Benzyl-2-butyl-6-chloro-8-(4-methyl-piperazin-1-yl)-1,1-dioxo-1,4-dihydro-2H-1λ⁶-benzo[1,2,4]thiadiazin-3-one,
(36) 4-Benzyl-6-chloro-2-cyclohexylmethyl-8-(4-methyl-piperazin-1-yl)-1,1-dioxo-1,4-dihydro-2H-1λ⁶-benzo[1,2,4]thiadiazin-3-one,
(37) 4-Benzyl-6-chloro-8-(4-methyl-piperazin-1-yl)-1,1-dioxo-2-phenethyl-1,4-dihydro-2H-1λ⁶-benzo[1,2,4]thiadiazin-3-one,
(38) 4-Benzyl-6-chloro-2-[3-(3,5-dimethyl-phenyl)-propyl]-8-(4-methyl-piperazin-1-yl)-1,1-dioxo-1,4-dihydro-2H-1λ⁶-benzo[1,2,4]thiadiazin-3-one,
(39) 4-Benzyl-6-chloro-8-(4-methyl-piperazin-1-yl)-2-octyl-1,1-dioxo-1,4-dihydro-2H-1λ⁶-benzo[1,2,4]thiadiazin-3-one,
(40) 4-Benzyl-6-chloro-2-decyl-8-(4-methyl-piperazin-1-yl)-1,1-dioxo-1,4-dihydro-2H-1λ⁶-benzo[1,2,4]thiadiazin-3-one,
(41) 4-Benzyl-6-chloro-8-(4-methyl-piperazin-1-yl)-2-naphthalen-1-ylmethyl-1,1-dioxo-1,4-dihydro-2H-1λ⁶-benzo[1,2,4]thiadiazin-3-one,
(42) 4-Benzyl-6-chloro-8-(4-methyl-piperazin-1-yl)-1,1-dioxo-2-pyridin-4-ylmethyl-1,4-dihydro-2H-1λ⁶-benzo[1,2,4]thiadiazin-3-one,
(43) 4-Benzyl-6-chloro-2-(5-methyl-furan-2-ylmethyl)-8-(4-methyl-piperazin-1-yl)-1,1-dioxo-1,4-dihydro-2H-1λ⁶-benzo[1,2,4]thiadiazin-3-one,
(44) 2,4-Dibenzyl-6-chloro-1,1-dioxo-8-piperazin-1-yl-1,4-dihydro-2H-1-benzo[1,2,4]thiadiazin-3-one,
(45) 4-Benzyl-6-chloro-2-(2-fluoro-benzyl)-1,1-dioxo-8-piperazin-1-yl-1,4-dihydro-2H-1λ⁶-benzo[1,2,4]thiadiazin-3-one,
(46) 4-Benzyl-6-chloro-2-(3-fluoro-benzyl)-1,1-dioxo-8-piperazin-1-yl-1,4-dihydro-2H-1λ⁶-benzo[1,2,4]thiadiazin-3-one,
(47) 4-Benzyl-6-chloro-2-(2-chloro-benzyl)-1,1-dioxo-8-piperazin-1-yl-1,4-dihydro-2H-1λ⁶-benzo[1,2,4]thiadiazin-3-one,
(48) 4-Benzyl-2-(2-bromo-benzyl)-6-chloro-1,1-dioxo-8-piperazin-1-yl-1,4-dihydro-2H-1λ⁶-benzo[1,2,4]thiadiazin-3-one,
(49) 4-Benzyl-6-chloro-2-(4-iodo-benzyl)-1,1-dioxo-8-piperazin-1-yl-1,4-dihydro-2H-1λ⁶-benzo[1,2,4]thiadiazin-3-one,
(50) 4-Benzyl-6-chloro-2-(2-methyl-benzyl)-1,1-dioxo-8-piperazin-1-yl-1,4-dihydro-2H-1λ⁶-benzo[1,2,4]thiadiazin-3-one,
(51) 4-Benzyl-6-chloro-2-(2-methoxy-benzyl)-1,1-dioxo-8-piperazin-1-yl-1,4-dihydro-2H-1λ⁶-benzo[1,2,4]thiadiazin-3-one,
(52) 4-Benzyl-6-chloro-2-(3-methoxy-benzyl)-1,1-dioxo-8-piperazin-1-yl-1,4-dihydro-2H-1λ⁶-benzo[1,2,4]thiadiazin-3-one,
(53) 4-Benzyl-6-chloro-2-(4-methoxy-benzyl)-1,1-dioxo-8-piperazin-1-yl-1,4-dihydro-2H-1λ⁶-benzo[1,2,4]thiadiazin-3-one,
(54) 4-Benzyl-6-chloro-2-(3-hydroxy-benzyl)-1,1-dioxo-8-piperazin-1-yl-1,4-dihydro-2H-1λ⁶-benzo[1,2,4]thiadiazin-3-one,
(55) 4-Benzyl-6-chloro-2-(2-nitro-benzyl)-1,1-dioxo-8-piperazin-1-yl-1,4-dihydro-2H-1λ⁶-benzo[1,2,4]thiadiazin-3-one,
(56) 4-(4-Benzyl-6-chloro-1,1,3-trioxo-8-piperazin-1-yl-3,4-dihydro-1H-1×-benzo[1,2,4]thiadiazin-2-ylmethyl)-benzoic acid methyl ester,
(57) 4-(4-Benzyl-6-chloro-1,1,3-trioxo-8-piperazin-1-yl-3,4-dihydro-1H-1λ⁶-benzo[1,2,4]thiadiazin-2-ylmethyl)-benzonitrile,
(58) 4-Benzyl-6-chloro-1,1-dioxo-2-[(R)-1-phenyl-ethyl]-8-piperazin-1-yl-1,4-dihydro-2H-1λ⁶-benzo[1,2,4]thiadiazin-3-one,
(59) 4-Benzyl-6-chloro-1,1-dioxo-2-[(S)-1-phenyl-ethyl]-8-piperazin-1-yl-1,4-dihydro-2H-1λ⁶-benzo[1,2,4]thiadiazin-3-one,
(60) 4-Benzyl-6-chloro-1,1-dioxo-2-phenyl-8-piperazin-1-yl-1,4-dihydro-2H-1λ⁶-benzo[1,2,4]thiadiazin-3-one,
(61) 4-Benzyl-6-chloro-2-(2-methoxy-phenyl)-1,1-dioxo-8-piperazin-1-yl-1,4-dihydro-2H-1λ⁶-benzo[1,2,4]thiadiazin-3-one,
(62) 4-Benzyl-6-chloro-2-(3-methoxy-phenyl)-1,1-dioxo-8-piperazin-1-yl-1,4-dihydro-2H-1λ⁶-benzo[1,2,4]thiadiazin-3-one,
(63) 4-Benzyl-6-chloro-2-(4-methoxy-phenyl)-1,1-dioxo-8-piperazin-1-yl-1,4-dihydro-2H-1λ⁶-benzo[1,2,4]thiadiazin-3-one,
(64) 4-Benzyl-6-chloro-2-ethyl-1,1-dioxo-8-piperazin-1-yl-1,4-dihydro-2H-1λ⁶-benzo[1,2,4]thiadiazin-3-one,
(65) 4-Benzyl-6-chloro-2-propyl-1,1-dioxo-8-piperazin-1-yl-1,4-dihydro-2H-1λ⁶-benzo[1,2,4]thiadiazin-3-one,
(66) 4-Benzyl-6-chloro-2-[3-(3,5-dimethyl-phenyl)-propyl]-1,1-dioxo-8-piperazin-1-yl-1,4-dihydro-2H-1λ⁶-benzo[1,2,4]thiadiazin-3-one,
(67) 4-Benzyl-6-chloro-2-decyl-1,1-dioxo-8-piperazin-1-yl-1,4-dihydro-2H-1λ⁶-benzo[1,2,4]thiadiazin-3-one,
(68) 4-Benzyl-6-chloro-2-naphthalen-1-ylmethyl-1,1-dioxo-8-piperazin-1-yl-1,4-dihydro-2H-1λ⁶-benzo[1,2,4]thiadiazin-3-one,
(69) 4-Benzyl-6-chloro-2-(2-chloro-benzyl)-8-morpholin-4-yl-1,1-dioxo-1,4-dihydro-2H-1λ⁶-benzo[1,2,4]thiadiazin-3-one,
(70) 4-Benzyl-6-chloro-2-(2-chloro-benzyl)-1,1-dioxo-8-(4-pyridin-2-yl-piperazin-1-yl)-1,4-dihydro-2H-1λ⁶-benzo[1,2,4]thiadiazin-3-one,
(71) 4-Benzyl-6-chloro-2-(2-chloro-benzyl)-8-[(R)-3-methyl-piperazin-1-yl]-1,1-dioxo-1,4-dihydro-2H-1λ⁶-benzo[1,2,4]thiadiazin-3-one,
(72) 4-Benzyl-6-chloro-2-(2-chloro-benzyl)-8-[(S)-3-methyl-piperazin-1-yl]-1,1-dioxo-1,4-dihydro-2H-1λ⁶-benzo[1,2,4]thiadiazin-3-one,
(73) 4-Benzyl-6-chloro-2-(2-chloro-benzyl)-8-[(6S)-1,4-diazabicyclo[4.3.0]non-4-yl]-1,1-dioxo-1,4-dihydro-2H-1λ⁶-benzo[1,2,4]thiadiazin-3-one,
(74) 4-Benzyl-6-chloro-2-(2-chloro-benzyl)-8-[(6R)-1,4-diazabicyclo[4.3.0]non-4-yl]-1,1-dioxo-1,4-dihydro-2H-1λ⁶-benzo[1,2,4]thiadiazin-3-one, and
(75) 4-Benzyl-6-chloro-2-(2-chloro-benzyl)-8-[(6R)-1,4-diazabicyclo[4.4.0]dec-4-yl]-1,1-dioxo-1,4-dihydro-2H-1λ⁶-benzo[1,2,4]thiadiazin-3-one.

However, the compounds of Formula 1 according to the present invention are not limited to the above-listed compounds.

Additionally, the present invention provides a preparation method of substituted-1,1-dioxo-benzo[1,2,4]thiadiazin-3-ones represented by Scheme 1 including the steps of:

(a) preparing an intermediate I by reaction of the compounds 2 and amine in the presence of a base;

(b) preparing an intermediate II by cyclization of the intermediate I;

(c) preparing an intermediate III by substitution reaction on intermediate II in the presence of a base; and

(d) preparing a compound of Formula 1 by nucleophilic substitution reaction of the intermediate III using a amine.

Additionally, depending on the R¹- and R²-substituents of the intermediate III or Formula 1, specific functional group transformations may be followed next the step (d) of the Scheme 1.

Hereinafter, a preparation method for the substituted-1,1-dioxo-benzo[1,2,4]thiadiazin-3-ones according to the present invention will be described in detail.

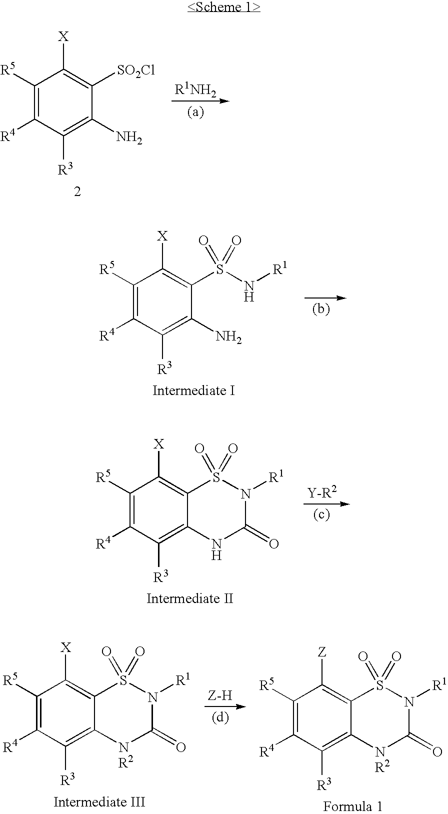

(wherein,

R¹˜R⁵ and Z are same as the aforementioned definition in Formula 1, and X is a fluorine, chlorine, bromine, iodine or trifluoroacetate, and Y is chlorine, bromine, iodine, methanesulfonate or p-toluenesulfonate.)

First, in the step (a), the intermediate I may be obtained by reaction of the compound 2 and a suitable amine in the presence of a base.

The compound 2 used as starting material, 2-aminosulfonylchlorides may be commercially available or where they are not commercially available, may be prepared by the procedure described herein or by the analogous procedures for known compounds from the art of organic synthesis. The base is preferably triethylamine and, the reaction is conveniently conducted in an inert solvent, such as 1,4-dioxane or tetrahydrofuran, at the room temperature.

Then, in the step (b), the cyclization of the intermediates I prepared in the step (a) provides the corresponding intermediates II (1,1-dioxo-1,4-dihydro-benzo[1,2,4]thiadiazin-3-ones) with high yield.

The cyclization is conducted by reaction of the intermediates I and phosgen (COCl₂), preferably, di-, tri-phosgen. The reaction is conveniently carried out in an inert solvent such as 1,4-dioxane or tetrahydrofuran under the refluxing condition.

Then, in the step (c), the intermediate III (2,4-substituted-benzo[1,2,4]thiadiazin-3-ones) is obtained by substitution on the intermediate II prepared in the above step (b) in the presence of a base.

Introduction of the substituent R² on N(4) of the intermediate II is usually carried out in the presence of a suitable base such as Na₂CO₃, K₂CO₃ or NaH in aprotic solvent such as acetonitrile, tetrahydrofuran, N,N-dimethylformamide etc. at ambient temperature. In this process, the leaving group Y preferably represents chlorine, bromine, iodine, methanesulfonate, or p-toluenesulfonate.

Subsequently, in the step (d), substituted-1,1-dioxo-benzo[1,2,4]thiadiazin-3-ones represented by formula 1 is obtained by neucleophilic substitution reaction of the intermediate III prepared in the step (c) and a appropriate amine.

Introduction of the amine moiety Z on C(8) of the formula I is carried out by a nucleophilic substitution reaction of the intermediate III, using appropriate amine such as piperazine, N-methylpiperazine, morpholine, 2-methylpiperazine, 1,4-diazepan-1-yl, octahydro-pyrido[1,2-a]pyrazine or octahydro-pyrrolo[1,2-a]pyrazine. This displacement is done using Na₂CO₃, K₂CO₃, triethylamine in aprotic solvent such as acetonitrile, N,N-dimethylformamide, in only basic solvent like pyridine, or in neat condition at reflux temperature.

And then, after the step (d), depending on the R¹- and R²-substituents of the intermediate III or Formula 1, specific functional group transformations may be performed.

A methoxy group may be transformed into a hydroxy group by treatment with a boron tribromide. A nitro (NO₂) group may be reduced to amino group using tin(II) dihydrate in refluxing protic solvent such as MeOH, EtOH and acetic acid or catalytic hydrogenation on palladium.

Where the above described processes for the preparation of the compounds according to the invention give rise to mixtures of stereoisomers, these isomers may be separated by conventional techniques such as preparative chromatography. The compounds may be prepared in racemic form, or individual enantiomers may be prepared either by asymmetric synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. The present invention extends to cover all structural and optical isomers of the various compounds as well as racemic mixture thereof.

Additionally, the present invention provides a pharmaceutical composition of a 5-HT6 antagonist including the compound of Formula 1, pharmaceutically acceptable salts thereof or a prodrug thereof.

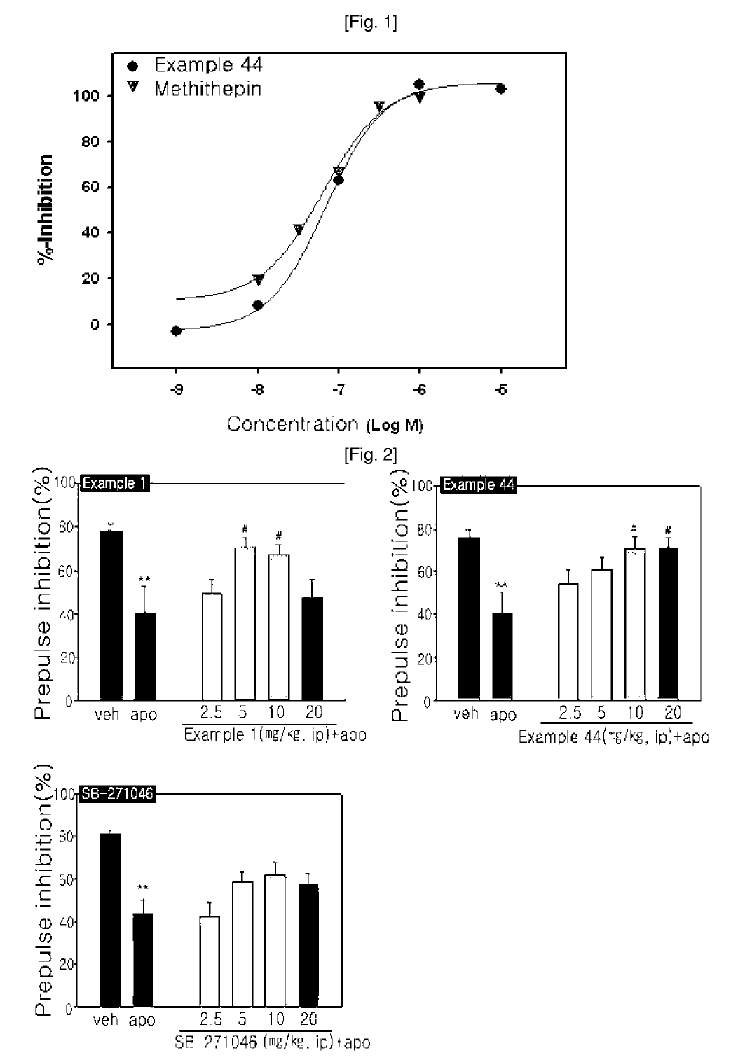

The compounds according to the present invention has excellent binding affinity to a serotonin 5-HT6 receptor (Refer to Table 2), excellent selectivity to a 5-HT6 receptor with respect to other receptors (Table 4), and the inhibitory effect on intracellular serotonin(5-HT)-induced cAMP accumulation (FIG. 1) and hyperactivity in rats induced by apomorphine (2 mg/kg, i.p.) (FIG. 2). In addition to, the compound according to the present invention don't show any rotarod deficit in effective dose. Therefore, it may be effectively used as a 5-HT6 antagonist.

The 5-HT6 receptor is known to be positively coupled to the adenylyl cyclase system, so agonists of the receptor would increase in a significant way the levels of intracellular cAMP. Thus a substance inhibiting the intracellular serotonin(5-HT)-induced cAMP accumulation may be determined as 5-HT6 receptor antagonist.

The 5-HT6 receptor is known to be positively coupled to the adenylyl cyclase system, so agonists of receptor would increase in a significant way the levels of intracellular cAMP. Thus, a substance inhibiting the intracellular serotonin(5-HT)-induced cAMP accumulation may be determined as a 5HT6 receptor antagonist.

Prepulse inhibition (PPI) of acoustic startle in animals for study the inhibitory effect on hyperactivity in rats is one of the most intensively studied behavioral models with predictive validity for antipsychotic properties of drugs. PPI is an occurrence that reduction or cease of the amplitude of the startle reaction when the main startle stimulus is preceded by the presentation of a weaker stimulus. PPI deficits have been reported in schizophrenic and presumably psychosis-prone subjects [Braff et al., 1992; Simons and Giardina, 1992].

Accordingly, a pharmaceutical composition according to the present invention may be used for treatment 5-HT6 receptor related disorders of the central nervous system, and particularly for cognitive disorders, Alzheimer disease, anxiety, depression, schizophrenia, stress disorder, panic disorder, phobic disorder, obsessive compulsive disorder, post-traumatic-stress syndrome, immune system depression, psychosis, paraphrenia, mania, convulsive disorder, migraine, drug addition, alcoholism, obesity, eating disorder, or sleep disorder.

The pharmaceutical compositions of this invention are preferably in unit dosage forms such as tablets, pills, capsules, powders, granules, sterile solutions or suspensions, or suppositories, for oral, intravenous, parenteral or rectal administration. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 □ of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixir and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

In the treatment of neurodegeneration, a suitable dosage level is about 0.01 to 250 □/□ per day, preferably about 0.05 to 100 □/□ per day, and especially about 0.05 to 5 □/□ per day. The compounds may be administered on a regimen of 1 to 4 times per day. In a particular embodiment, the compounds may be conveniently administered by intravenous infusion.

The following examples are illustrative, but not limiting, of the method and compositions of the present invention. Other suitable modification and adaption of the variety of conditions and parameters normally encountered in clinical therapy and which are obvious to those skilled in the art are within the spirit and scope of the invention.

### MODE FOR THE INVENTION

The following Intermediates and Examples illustrate the preparation of compounds of the invention.

### Preparation Example 1

### Preparation of 2-amino-4,6-dichloro-benzenesulfonyl chloride

A mixture of 3,5-dichloro aniline (1.00 g, 6.20 mmol) and chlorosulfonic acid (6□). was stirred at reflux temperature. After the reaction was completed, the mixture was poured into ice water. The resulting solid was filtered and washed with cold water several times to provide the crude benzenesulfonyl chloride as a gray solid in 74% (1.19 g) yield: ¹H NMR (200 MHz, CDCl₃) δ 6.71 (d, J=2.0 Hz, 1H, ArH), 6.87 (d, J=2.0 Hz, 1H, ArH), 8.21 (br s, 2H, NH₂); MS(EI) m/e 259[M⁺], 160, 124.

### Preparation Example 2

### General Procedure for the synthesis of N-substituted-benzenesulfonamides (Intermediate I)

To a solution of 2-amino-4,6-dichloro-benzenesulfonyl chloride (2.0 mmol) in 1,4-dioxane (25□) was added the suitable amine (2.4 mmol) and triethylamine (3.0 mmol) at room temperature. The resulting mixture was stirred for 5 hours at ambient temperature. After the starting benzenesulfonyl chloride disappeared, the solvent was removed under reduced pressure. The residues was dissolved with ethyl acetate and washed with 0.5 M HCl aqueous solution, water and brine. The organic layer was dried over anhydrous MgSO₄ and concentrated in vacuo. The crude was purified by a flash column chromatography (eluent; a mixture of n-hexane and ethyl acetate) to give the correspoding N-substituted-benzenesulfonamides (Intermediate I).

### Preparation Example 2-1

### 2-Amino-N-benzyl-4,6-dichloro-benzenesulfonamide (Intermediate I-1)

(yield, 73%), white solid; m.p. 125-126° C.; ¹H NMR (200 MHz, CDC₃) δ 4.14 (d, J=6.6 Hz, 2H, NCH₂Ar), 5.44 (t, J=6.6 Hz, 1H, NH), 5.72 (br s, 2H, NH₂), 6.61 (d, J=2.0 Hz, 1H, ArH), 6.73 (d, J=2.0 Hz, 1H, ArH), 7.25-7.30 (m, 5H, ArH); MS(EI) m/e 330 [M⁺].

### Preparation Example 2-2

### 2-Amino-4,6-dichloro-N-(2-fluoro-benzyl)-benzenesulfonamide (Intermediate I-2)

(yield, 97%), bright yellow solid; m.p. 97-98° C.; ¹H NMR (200 MHz, CDCl₃) δ 4.22 (d, J=6.4 Hz, 2H, NCH₂Ar), 5.66-5.68 (m, 3H, NH & NH), 6.50 (d, J=2.0 Hz, 1H, ArH), 6.57 (d, J=2.0 Hz, 1H, ArH), 6.91-7.00 (m, 2H, ArH), 7.16-7.27 (m, 2H, ArH); MS(EI) m/e 348 [M⁺].

### Preparation Example 2-3

### 2-Amino-4,6-dichloro-N-(3-fluoro-benzyl)-benzenesulfonamide (Intermediate I-3)

(yield, 98%), pale yellow solid; m.p. 82-83° C.; ¹H NMR (200 MHz, CDCl₃) δ 4.11 (d, J=6.0 Hz, 2H, NCH₂Ar), 5.65 (t, J=6.0 Hz, 1H, NH), 5.70 (br s, 2H, NH₂), 6.57 (d, J=2.0 Hz, 1H, ArH), 6.66 (d, J=2.0 Hz, 1H, ArH), 6.89-7.03 (m, 3H, ArH), 7.22 (m, 1H, ArH); MS(EI) m/e 348 [M⁺].

### Preparation Example 2-4

### 2-Amino-4,6-dichloro-N-(4-fluoro-benzyl)-benzenesulfonamide (Intermediate I-4)

(yield, 97%), pale yellow solid; m.p. 98-99° C.; ¹H NMR (200 MHz, CDC₃) δ 4.09 (d, J=6.2 Hz, 2H, NCH₂Ar), 5.40 (t, J=6.2 Hz, 1H, NH), 5.67 (br s, 2H, NH₂), 6.58 (d, J=2.0 Hz, 1H, ArH), 6.71 (d, J=2.0 Hz, 1H, ArH), 6.92-7.02 (m, 2H, ArH), 7.18-7.23 (m, 2H, ArH); MS(EI) m/e 348 [M⁺].

### Preparation Example 2-5

### 2-Amino-4,6-dichloro-N-(2-chloro-benzyl)-benzenesulfonamide (Intermediate I-5)

(yield, 98%), pale brown solid; m.p. 93-94° C.; ¹H NMR (200 MHz, CDCl₃) δ 4.30 (d, J=6.6 Hz, 2H, NCH₂Ar), 5.69 (br s, 2H, NH₂), 5.79 (br t, J=6.6 Hz, 1H, NH), 6.54 (d, J=2.2 Hz, 1H, ArH), 6.59 (d, J=2.2 Hz, 1H, ArH), 7.06-7.35 (m, 4H, ArH); MS(EI) m/e 365 [M⁺+1].

### Preparation Example 2-6

### 2-Amino-4,6-dichloro-N-(3-chloro-benzyl)-benzenesulfonamide (Intermediate I-6)

(yield, 98%), pale yellow solid; m.p. 108-109° C.; ¹H NMR (200 MHz, CDCl₃) δ 4.12 (d, J=6.4 Hz, 2H, NCH₂Ar), 5.49 (br t, J=6.4 Hz, 1H, NH), 5.67 (br s, 2H, NH₂), 6.56 (d, J=2.0 Hz, 1H, ArH), 6.68 (d, J=2.0 Hz, 1H, ArH), 7.11-7.22 (m, 4H, ArH); MS(EI) m/e 365 [M⁺+1].

### Preparation Example 2-7

### 2-Amino-4,6-dichloro-N-(4-chloro-benzyl)-benzenesulfonamide (Intermediate I-7)

(yield, ˜quantitative), yellow solid; m.p. 84-85° C.; ¹H NMR (200 MHz, CDCl₃) δ 4.09 (d, J=6.2 Hz, 2H, NCH₂Ar), 5.44 (br t, J=6.2 Hz, 1H, NH), 5.67 (br s, 2H, NH₂), 6.58 (d, J=2.0 Hz, 1H, ArH), 6.71 (d, J=2.0 Hz, 1H, ArH), 7.16-7.27 (m, 4H, ArH); MS(EI) m/e 364 [M⁺].

### Preparation Example 2-8

### 2-Amino-N-(2-bromo-benzyl)-4,6-dichloro-benzenesulfonamide (Intermediate I-8)

(yield, 96%), yellow solid; m.p. 109-110° C.; ¹H NMR (200 MHz, CDCl₃) δ 4.29 (d, J=6.8 Hz, 2H, NCH₂Ar), 5.71 (br s, 2H, NH₂), 5.84 (br t, J=6.8 Hz, 1H, NH), 6.55 (d, J=2.0 Hz, 1H, ArH), 6.58 (d, J=2.0 Hz, 1H, ArH), 7.11-7.18 (m, 2H, ArH), 7.22 (m, 1H, ArH), 7.50 (m, 1H, ArH); MS(EI) m/e 409 [M⁺+1].

### Preparation Example 2-9

### 2-Amino-N-(3-bromo-benzyl)-4,6-dichloro-benzenesulfonamide (Intermediate I-9)

(yield, 98%), yellow solid; m.p. 94-96° C.; ¹H NMR (200 MHz, CDCl₃) δ 4.11 (d, J=6.4 Hz, 2H, NCH₂Ar), 5.53 (br t, J=6.4 Hz, 1H, NH), 5.66 (br s, 2H, NH₂), 6.56 (d, J=2.0 Hz, 1H, ArH), 6.67 (d, J=2.0 Hz, 1H, ArH), 7.12-7.16 (m, 2H, ArH), 7.35-7.39 (m, 2H, ArH); MS(EI) m/e 409 [M⁺+1].

### Preparation Example 2-10

### 2-Amino-N-(4-bromo-benzyl)-4,6-dichloro-benzenesulfonamide (Intermediate I-10)

(yield, 97%), yellow solid; m.p. 108-109° C.; ¹H NMR (200 MHz, CDCl₃) δ 4.07 (d, J=6.2 Hz, 2H, NCH₂Ar), 5.49 (br t, J=6.2 Hz, 1H, NH), 5.68 (br s, 2H, NH₂), 6.58 (d, J=2.0 Hz, 1H, ArH), 6.70 (d, J=2.0 Hz, 1H, ArH), 7.10-7.15 (m, 2H, ArH), 7.36-7.43 (m, 2H, ArH); MS(EI) m/e 409 [M⁺+1].

### Preparation Example 2-11

### 2-Amino-4,6-dichloro-N-(3-iodo-benzyl)-benzenesulfonamide (Intermediate I-11)

(yield, ˜quantitative), yellow solid; m.p. 97-98° C.; ¹H NMR (200 MHz, CDCl₃) δ 4.09 (d, J=6.6 Hz, 2H, NCH₂Ar), 5.49 (br t, J=6.6 Hz, 1H, NH), 5.65 (br s, 2H, NH₂), 6.55 (d, J=2.0 Hz, 1H, ArH), 6.66 (d, J=2.0 Hz, 1H, ArH), 6.94-7.02 (m, 1H, ArH), 7.18-7.26 (m, 1H, ArH), 7.54-7.58 (m, 2H, ArH); MS(EI) m/e 455 [M⁺].

### Preparation Example 2-12

### 2-Amino-4,6-dichloro-N-(4-iodo-benzyl)-benzenesulfonamide (Intermediate I-12)

(yield, 95%), white solid; m.p. 105-108° C.; ¹H NMR (200 MHz, CDCl₃CD₃OD) δ 4.07 (s, 2H, NCH₂Ar), 6.63 (d, J=2.0 Hz, 1H, ArH), 6.99-7.08 (m, 2H, ArH), 7.03 (d, J=2.0 Hz, 1H, ArH), 7.60-7.69 (m, 2H, ArH); MS(EI) m/e 457 [M⁺+1].

### Preparation Example 2-13

### 2-Amino-4,6-dichloro-N-(2-methyl-benzyl)-benzenesulfonamide (Intermediate I-13)

(yield, 97%), white solid; m.p. 134-136° C.; ¹H NMR (200 MHz, CDCl₃) δ 2.34 (s, 3H, CH₃), 4.09 (d, J=6.2 Hz, 2H, NCH₂Ar), 5.22 (br t, J=6.2 Hz, 1H, NH), 5.71 (br s, 2H, NH₂), 6.61 (d, J=2.0 Hz, 1H, ArH), 6.73 (d, J=2.0 Hz, 1H, ArH), 7.10-7.22 (m, 4H, ArH); MS(EI) m/e 344 [M⁺], 161, 224.

### Preparation Example 2-14

### 2-Amino-4,6-dichloro-N-(3-methyl-benzyl)-benzenesulfonamide (Intermediate I-14)

(yield, 95%), white solid; m.p. 131-134° C.; ¹H NMR (200 MHz, CDC₃) δ 2.29 (s, 3H, CH₃), 4.09 (d, J=6.2 Hz, 2H, NCH₂Ar), 5.40 (br t, J=6.2 Hz, 1H, NH), 5.68 (br s, 2H, NH₂), 6.57 (d, J=2.0 Hz, 1H, ArH), 6.68 (d, J=2.0 Hz, 1H, ArH), 7.02-7.21 (m, 4H, ArH); MS(EI) m/e 344 [M⁺], 105, 161.

### Preparation Example 2-15

### 2-Amino-4,6-dichloro-N-(4-methyl-benzyl)-benzenesulfonamide (Intermediate I-15)

(yield, 91%), m.p. 102-105° C.; ¹H NMR (200 MHz, CDCl₃) δ 2.31 (s, 3H, CH₃), 4.07 (d, J=6.2 Hz, 2H, NCH₂Ar), 5.35 (br t, J=6.2 Hz, 1H, NH), 5.68 (br s, 2H, NH₂), 6.57 (d, J=2.0 Hz, 1H, ArH), 6.70 (d, J=2.0 Hz, 1H, ArH), 7.05-7.15 (m, 4H, ArH); MS(EI) m/e 344 [M⁺], 161.

### Preparation Example 2-16

### 2-Amino-4,6-dichloro-N-(2-methoxy-benzyl)-benzenesulfonamide (Intermediate I-16)

(yield, 82%), sticky oil; ¹H NMR (200 MHz, CDCl₃) δ 3.80 (s, 3H, OCH₃), 4.19 (d, J=6.6 Hz, 2H, NCH₂Ar), 5.64 (br s, 2H, NH₂), 5.99 (t, J=6.6 Hz, 1H, NH), 6.42 (d, J=2.2 Hz, 1H, ArH), 6.46 (d, J=2.2 Hz, 1H, ArH), 6.66-6.74 (m, 2H, ArH), 7.02 (m, 1H, ArH), 7.17 (m, 1H, ArH); MS(EI) m/e 362 [M⁺+2], 360 [M⁺].

### Preparation Example 2-17

### 2-Amino-4,6-dichloro-N-(3-methoxy-benzyl)-benzenesulfonamide (Intermediate I-17)

(yield, 76%), pale violet solid; ¹H NMR (200 MHz, CDCl₃) δ 3.77 (s, 3H, OCH₃), 4.09 (d, J=6.6 Hz, 2H, NCH₂Ar), 5.44 (t, J=6.6 Hz, 1H, NH), 5.69 (br s, 2H, NH₂), 6.57 (d, J=1.2 Hz, 1H, ArH), 6.68 (d, J=1.2 Hz, 1H, ArH), 6.77-6.83 (m, 3H, ArH), 7.15 (dd, J=7.8, 8.0 Hz, 1H, ArH).

### Preparation Example 2-18

### 2-Amino-4,6-dichloro-N-(4-methoxy-benzyl)-benzenesulfonamide (Intermediate I-18)

(yield, 87%), sticky oil; ¹H NMR (200 MHz, CDC₃) δ 3.81 (s, 3H, OCH₃), 4.10 (s, 2H, NCH₂Ar), 5.42 (br s, 1H, NH), 5.72 (br s, 2H, NH₂), 6.59 (d, J=2.2 Hz, 1H, ArH), 6.72 (d, J=2.2 Hz, 1H, ArH), 6.80-6.84 (m, 2H, ArH), 7.15-7.19 (m, 2H, ArH).

### Preparation Example 2-19

### 2-Amino-4,6-dichloro-N-(2-nitro-benzyl)-benzenesulfonamide (Intermediate I-19)

(yield, 98%), yellow solid; m.p. 116-117° C.; ¹H NMR (200 MHz, CDCl₃) δ 4.49 (d, J=7.0 Hz, 2H, NCH₂Ar), 5.66 (br s, 2H, NH₂), 6.22 (br t, J=7.0 Hz, 1H, NH), 6.50 (d, J=2.0 Hz, 1H, ArH), 6.52 (d, J=2.0 Hz, 1H, ArH), 7.38-7.45 (m, 3H, ArH), 8.05 (m, 1H, ArH); MS(EI) m/e 374 [M⁺].

### Preparation Example 2-20

### 2-Amino-4,6-dichloro-N-(3-nitro-benzyl)-benzenesulfonamide (Intermediate I-20)

(yield, 96%), yellow solid; m.p. 102-106° C.; ¹H NMR (200 MHz, CDCl₃) δ 4.27 (d, J=6.6 Hz, 2H, NCH₂Ar), 5.66 (br s, 1H, NH), 6.55 (d, J=2.0 Hz, 1H, ArH), 6.67 (d, J=2.0 Hz, 1H, ArH), 7.48 (m, 1H, ArH), 7.63 (m, 1H, ArH), 8.10-8.13 (m, 2H, ArH); MS(EI) 151, 161 m/e 375[M⁺].

### Preparation Example 2-21

### 2-Amino-4,6-dichloro-N-(4-nitro-benzyl)-benzenesulfonamide (Intermediate I-21)

(yield, ˜quantitative), yellow solid; m.p. 103-105° C.; ¹H NMR (200 MHz, CDCl₃+CD₃OD) δ 4.20 (s, 2H, NCH₂Ar), 6.57 (d, J=2.0 Hz, 1H, ArH), 6.67 (d, J=2.0 Hz, 1H, ArH), 7.42-7.46 (m, 2H, ArH), 8.08-8.14 (m, 2H, ArH); MS(EI) m/e 375 [M⁺].

### Preparation Example 2-22

### 4-[(2-Amino-4,6-dichloro-benzenesulfonylamino)-methyl]-benzoic acid methyl ester (Intermediate I-22)

(yield, 89%), white solid; m.p. 151-157° C.; ¹H NMR (200 MHz, CDC₃) δ 3.91 (s, 3H, OCH₃), 4.18 (d, J=6.4 Hz, 2H, NCH₂Ar), 5.49 (br t, J=6.2 Hz, 1H, NH), 5.66 (br s, 2H, NH₂), 6.57 (d, J=2.0 Hz, 1H, ArH), 6.71 (d, J=2.0 Hz, 1H, ArH), 7.31-7.35 (m, 2H, ArH), 7.93-7.97 (m, 2H, ArH); MS(EI) m/e 388 [M⁺].

### Preparation Example 2-23

### 2-Amino-4,6-dichloro-N-(4-cyano-benzyl)-benzenesulfonamide (Intermediate I-23)

(yield, 98%), white solid; m.p. 168-169° C.; ¹H NMR (200 MHz, CDC₃) δ 4.18 (d, J=6.6 Hz, 2H, NCH₂Ar), 5.54 (br t, J=6.6 Hz, 1H, NH), 5.66 (br s, 2H, NH₂), 6.59 (d, J=2.0 Hz, 1H, ArH), 6.73 (d, J=2.0 Hz, 1H, ArH), 7.38-7.42 (m, 2H, ArH), 7.58-7.62 (m, 2H, ArH); MS(EI) m/e 355 [M⁺].

### Preparation Example 2-24

### 2-Amino-4,6-dichloro-N—((R)-1-phenyl-ethyl)-benzenesulfonamide (Intermediate I-24)

(yield, 72%), pale yellow solid; ¹H NMR (200 MHz, CDCl₃) δ 1.50 (d, J=7.0 Hz, 3H, CH₃), 4.45 (m, 1H, NCHMeAr), 5.52 (d, J=7.2 Hz, 1H, NH), 5.70 (br s, 2H, NH2), 6.48 (d, J=2.2 Hz, 1H, ArH), 6.62 (d, J=2.2 Hz, 1H, ArH), 7.24 (m, 5H, ArH); MS(EI) m/e 345 [M⁺+1].

### Preparation Example 2-25

### 2-Amino-4,6-dichloro-N—((S)-1-phenyl-ethyl)-benzenesulfonamide (Intermediate I-25)

(yield, 74%), pale yellow solid; ¹H NMR (200 MHz, CDCl₃) δ 1.52 (d, J=6.8 Hz, 3H, CH₃), 4.45 (m, 1H, NCHMeAr), 5.48 (d, J=6.8 Hz, 1H, NH), 5.68 (br s, 2H, NH₂), 6.47 (d, J=2.2 Hz, 1H, ArH), 6.68 (d, J=2.2 Hz, 1H, ArH), 7.21 (m, 4H, ArH), 7.34 (m, 1H, ArH); MS(EI) m/e 344 [M⁺].

### Preparation Example 2-26

### 2-Amino-4,6-dichloro-N-phenyl-benzenesulfonamide (Intermediate I-26)

(yield, 75%), white solid; ¹H NMR (200 MHz, CDC₃) δ 5.39 (br s, 2H, NH₂), 6.51 (d, J=2.0 Hz, 1H, ArH), 6.70 (d, J=2.0 Hz, 1H, ArH), 7.09-7.17 (m, 3H, ArH), 7.18 (br s, 1H, NH), 7.23-7.31 (m, 2H, ArH); MS(EI) m/e 317 [M⁺+1].

### Preparation Example 2-27

### 2-Amino-4,6-dichloro-N-(2-methoxy-phenyl)-benzenesulfonamide (Intermediate I-27)

(yield, 67%), white solid; ¹H NMR (200 MHz, CDCl₃) δ 3.79 (s, 3H, OCH₃), 5.66 (br s, 2H, NH₂), 6.51 (d, J=2.0 Hz, 1H, ArH), 6.69 (d, J=2.0 Hz, 1H, ArH), 6.80-6.90 (m, 2H, ArH), 7.05 (m, 1H, ArH), 7.35 (m, 1H, ArH), 7.73 (br s, 1H, NH); MS(EI) m/e 346 [M⁺].

### Preparation Example 2-28

### 2-Amino-4,6-dichloro-N-(3-methoxy-phenyl)-benzenesulfonamide (Intermediate I-28)

(yield, 83%), pale yellow solid; m.p. 134-135° C.; ¹H NMR (200 MHz, CDCl₃) δ 3.75 (s, 3H, OCH₃), 5.65 (br s, 2H, NH₂), 6.52 (d, J=2.0 Hz, 1H, ArH), 6.62-6.69 (m, 2H, ArH), 6.71 (d, J=2.0 Hz, 1H, ArH), 7.11-7.19 (m, 2H, ArH); MS(EI) m/e 346 [M⁺], 284, 160.

### Preparation Example 2-29

### 2-Amino-4,6-dichloro-N-(4-methoxy-phenyl)-benzenesulfonamide (Intermediate I-29)

(yield, 72%), white solid; ¹H NMR (200 MHz, CDCl₃) δ 3.76 (s, 3H, OCH₃), 6.48 (m, 1H, ArH), 6.75-6.80 (m, 2H, ArH), 6.98-7.08 (m, 3H, ArH); MS(EI) m/e 346 [M⁺⁺1].

### Preparation Example 2-30

### 2-Amino-4,6-dichloro-N-methyl-benzenesulfonamide (Intermediate I-30)

(yield, 86%), white solid; ¹H NMR (200 MHz, CDC₃) δ 3.04 (d, J=6.4 Hz, 3H, NCH₃), 5.12 (br s, 1H, NH), 5.62 (br s, 2H, NH₂), 6.49 (d, J=2.2 Hz, 1H, ArH), 6.70 (d, J=2.2 Hz, 1H, ArH); MS(EI) m/e 254 [M⁺], 240.

### Preparation Example 2-31

### 2-Amino-4,6-dichloro-N-ethyl-benzenesulfonamide (Intermediate I-31)

(yield, 94%), white solid; m.p. 175-177° C.; ¹H NMR (200 MHz, CDC₃) δ 1.03 (t, J=6.8 Hz, 3H, CH₃), 2.95 (m, 2H, NCH₂), 5.10 (br t, J=5.4 Hz, 1H, NH), 5.70 (br s, 2H, NH₂), 6.56 (d, J=2.0 Hz, 1H, ArH), 6.66 (d, J=2.0 Hz, 1H, ArH); MS(EI) m/e 268 [M⁺], 176, 161.

### Preparation Example 2-32

### 2-Amino-4,6-dichloro-N-propyl-benzenesulfonamide (Intermediate I-32)

(yield, 94%), white solid; ¹H NMR (200 MHz, CDC₃) δ 0.88 (t, J=7.2 Hz, 3H, CH₃), 1.45-1.63 (m, 2H, CH₂), 2.86-2.97 (m, 2H, NCH₂), 5.19 (br t, J=7.2 Hz, 1H, NH), 5.76 (br s, 2H, NH₂), 6.64 (d, J=2.0 Hz, 1H, ArH), 6.75 (d, J=2.0 Hz, 1H, ArH); MS(EI) m/e 282 [M⁺], 224, 162.

### Preparation Example 2-33

### 2-Amino-N-butyl-4,6-dichloro-benzenesulfonamide (Intermediate I-33)

(yield, 94%), white solid; m.p. 117-119° C.; ¹H NMR (200 MHz, CDC₃) δ 0.85 (t, J=7.0 Hz, 3H, CH₃), 1.32 (m, 2H, CH₂), 1.48 (m, 2H, CH₂), 2.96 (m, 2H, NCH₂), 5.08 (br t, J=6.8 Hz, 1H, NH), 5.70 (br s, 2H, NH₂), 6.62 (d, J=2.0 Hz, 1H, ArH), 6.76 (d, J=2.0 Hz, 1H, ArH); MS(EI) m/e 296 [M⁺], 224, 176.

### Preparation Example 2-34

### 2-Amino-4,6-dichloro-N-cyclo-hexylmethyl-benzenesulfonamide (Intermediate I-34)

(yield, 80%), pale yellow solid; m.p. 103-106° C.; ¹H NMR (200 MHz, CDCl₃) δ 0.81-1.74 (m, 11H, CH₂×5 and CH of cyclohexyl group), 2.70 (t, J=6.6 Hz, 2H, NHCH₂), 5.12 (br t, J=6.0 Hz, 1H, NH), 5.70 (s, 2H, NH 2) 6.62 (d, J=2.0 Hz, 1H, ArH), 6.76 (d, J=2.0 Hz, 1H, ArH); MS(EI) m/e 336 [M⁺], 253, 240, 161.

### Preparation Example 2-35

### 2-Amino-4,6-dichloro-N-phenethyl-benzenesulfonamide (Intermediate I-35)

(yield, 99%), white solid; ¹H NMR (200 MHz, CDC₃) δ 2.83 (t, J=6.9 Hz, 2H, CH₂Ar), 3.22 (m, 2H, NCH₂), 5.15 (m, 1H, NH), 5.70 (s, 2H, NH₂), 6.60 (d, J=2.2 Hz, 1H, ArH), 6.69 (d, J=2.2 Hz, 1H, ArH), 7.11-7.15 (m, 2H, ArH), 7.23-7.34 (m, 3H, ArH); MS(EI) m/e 344 [M⁺].

### Preparation Example 2-36

### 2-Amino-4,6-dichloro-N-[3-(3,5-dimethyl-phenyl)-propyl]-benzenesulfonamide (Intermediate I-36)

(yield, 84%), pale yellow solid; ¹H NMR (200 MHz, CDC₃) δ 1.76-1.87 (m, 2H, CH2), 2.26 (s, 6H, CH₃×2), 2.55 (t, J=6.5 Hz, 2H, CH₂Ar), 2.96 (m, 2H, NCH₂), 5.15 (t, J=6.4 Hz, 1H, NH), 5.67 (br s, 2H, NH₂), 6.60 (d, J=2.2 Hz, 1H, ArH), 6.71 (d, J=3.0 Hz, 2H, ArH), 6.76 (d, J=2.2 Hz, 1H, ArH), 6.82 (d, J=3.0 Hz, 1H, ArH).

### Preparation Example 2-37

### 2-Amino-4,6-dichloro-N-octyl-benzenesulfonamide (Intermediate I-37)

(yield, 94%), pale yellow solid; ¹H NMR (200 MHz, CDCl₃) δ 0.83 (t, J=6.6 Hz, 3H, CH₃), 1.23-1.34 (m, 10H, CH₂×5), 1.48 (m, 2H, CH₂), 2.94 (t, J=6.2 Hz, 2H, NCH₂) 5.20 (br t, J=6.2 Hz, 1H, NH), 5.79 (br s, 2H, NH₂), 6.64 (d, J=2.0 Hz, 1H, ArH), 6.73 (d, J=2.0 Hz, 1H, ArH); MS(EI) m/e 352[M⁺], 240, 224.

### Preparation Example 2-38

### 2-Amino-4,6-dichloro-N-decyl-benzenesulfonamide (Intermediate I-38)

(yield, 97%), dark brown oil; ¹H NMR (200 MHz, CDC₃) δ 0.84 (t, J=6.4 Hz, 3H, CH₃ of decyl), 1.23-1.53 (m, 16H, CH₂×8), 2.93 (m, 2H, NCH₂), 5.07 (br t, J=6.2 Hz, 1H, NH), 5.70 (br s, 2H, NH₂), 6.61 (d, J=2.0 Hz, 1H, ArH), 6.75 (d, J=2.0 Hz, 1H, ArH); MS(EI) m/e 380 [M⁺].

### Preparation Example 2-39

### 2-Amino-4,6-dichloro-N-(naphthalen-1-ylmethyl)-benzenesulfonamide (Intermediate I-39)

(yield, 96%), pale yellow solid; m.p. 180-181° C.; ¹H NMR (200 MHz, CDCl₃+CD₃OD) δ 4.58 (s, 2H, NCH₂Ar), 6.52 (d, J=2.0 Hz, 1H, ArH), 6.59 (d, J=2.0 Hz, 1H, ArH), 7.34-7.55 (m, 4H, ArH), 7.76-7.87 (m, 2H, ArH), 8.02 (m, 1H, ArH); MS(EI) m/e 380 [M⁺].

### Preparation Example 2-40

### 2-Amino-4,6-dichloro-N-pyridin-4-ylmethyl-benzenesulfonamide (Intermediate I-40)

(yield, 79%), yellow solid; m.p. 251-252° C.; ¹H NMR (200 MHz, DMSO) δ 4.34 (d, J=6.6 Hz, 2H, NCH₂Ar), 6.74 (d, J=2.0 Hz, 1H, ArH), 6.85 (d, J=2.0 Hz, 1H, ArH), 7.85-7.88 (m, 2H, ArH), 7.78-8.81 (m, 2H, ArH), 6.73-6.87 (m, 3H, NH₂ & NH); MS(EI) m/e 331[M⁺].

### Preparation Example 2-41

### 2-Amino-4,6-dichloro-N-(5-methyl-furan-2-ylmethyl)-benzenesulfonamide (Intermediate I-41)

(yield, 95%), yellow solid; m.p. 109-110° C.; ¹H NMR (200 MHz, CDC₃) δ 2.24 (s, 3H, CH₃), 4.14 (d, J=6.2 Hz, 2H, NCH₂Ar), 5.60 (br t, J=6.2 Hz, 1H, NH), 5.69-5.71 (m, 3H, NH₂, CH of furanyl), 5.97 (d, J=2.8 Hz, 1H, CH of furanyl), 6.53 (d, J=2.0 Hz, 1H, ArH), 6.65 (d, J=2.0 Hz, 1H, ArH); MS(EI) m/e 334 [M⁺].

### Preparation Example 3

### General Procedure for the synthesis of 2-Substituted-1,1-dioxo-1,4-dihydro-2H-1λ⁶-benzo[1,2,4]thiadiazin-3-ones (Intermediate II)

The appropriate intermediate I (1.0 mmol) in 1,4-dioxane (20□) was treated with triphosgen (0.4 mmol) at reflux temperature for 2 hours. The solvent was removed under reduced pressure. The residues was dissolved with ethyl acetate and washed with 0.5 M HCl aqueous solution, water and brine. The organic layer was dried over anhydrous MgSO₄ and concentrated in vacuo. The crude was purified by a flash column chromatography (eluent; a mixture of n-hexane and ethyl acetate) to produce the correspoding 2-substituted-benzo[1,2,4]thiadiazin-3-ones (Intermediate II).

### Preparation Example 3-1

### 2-Benzyl-6,8-dichloro-1,1-dioxo-1,4-dihydro-2H-1λ⁶-benzo[1,2,4]thiadiazin-3-one (Intermediate II-1)

(yield, 78%), light gray solid; m.p. 198-200° C.; ¹H NMR (200 MHz, CDC₃) δ 5.11 (s, 2H, NCH₂Ar), 6.95 (d, J=2.0 Hz, 1H, ArH), 7.28-7.41 (m, 4H, ArH), 7.49-7.54 (m, 2H, ArH), 9.79 (br s, 1H, NH); MS(EI) m/e 356 [M⁺].

### Preparation Example 3-2

### 6,8-Dichloro-2-(2-fluoro-benzyl)-1,1-dioxo-1,4-dihydro-2H-1λ⁶-benzo[1,2,4]thiadiazin 3-one (Intermediate II-2)

(yield, 91%), pale gray solid; m.p. 204-206° C.; ¹H NMR (200 MHz, CDCl₃+CD₃OD) δ 5.16 (s, 2H, NCH₂Ar), 6.99-7.14 (m, 3H, ArH), 7.23-7.45 (m, 3H, ArH); MS(EI) m/e 374 [M⁺].

### Preparation Example 3-3

### 6,8-Dichloro-2-(3-fluoro-benzyl)-1,1-dioxo-1,4-dihydro-2H-1λ⁶-benzo[1,2,4]thiadiazin-3-one (Intermediate II-3)

(yield, 94%), pale gray solid; m.p. 176-178° C.; ¹H NMR (200 MHz, CDCl₃+CD₃OD) δ 5.03 (s, 2H, NCH₂Ar), 6.96 (m, 1H, ArH), 7.11 (m, 1H, ArH), 7.16-7.34 (m, 4H, ArH); MS(EI) m/e 374 [M⁺].

### Preparation Example 3-4

### 6,8-Dichloro-2-(4-fluoro-benzyl)-1,1-dioxo-1,4-dihydro-2H-1λ⁶-benzo[1,2,4]thiadiazin-3-one (Intermediate II-4)

(yield, 93%), pale gray solid; m.p. 222-224° C.; ¹H NMR (200 MHz, CDCl₃+CD₃OD) δ 5.02 (s, 2H, NCH₂Ar), 6.96-7.08 (m, 3H, ArH), 7.22 (m, 1H, ArH), 7.45-7.52 (m, 2H, ArH); MS(EI) m/e 374 [M⁺].

### Preparation Example 3-5

### 2-Benzyl-6,8-dichloro-1,1-dioxo-1,4-dihydro-2H-1λ⁶-benzo[1,2,4]thiadiazin-3-one (Intermediate II-5)

(yield, 90%), white solid; m.p. 218-219° C.; ¹H NMR (200 MHz, CDC₃) δ 5.26 (s, 2H, NCH₂Ar), 6.97 (d, J=1.6 Hz, 1H, ArH), 7.20-7.42 (m, 5H, ArH), 9.87 (br s, 1H, NH); MS(EI) m/e 390 [M⁺].

### Preparation Example 3-6

### 6,8-Dichloro-2-(3-chloro-benzyl)-1,1-dioxo-1,4-dihydro-2H-1λ⁶-benzo[1,2,4]thiadiazin-3-one (Intermediate II-6)

(yield, 92%), pale yellow solid; m.p. 181-182° C.; ¹H NMR (200 MHz, CDCl₃) δ 5.05 (s, 2H, NCH₂Ar), 6.94 (d, J=1.8 Hz, 1H, ArH), 7.26-7.49 (m, 5H, ArH), 9.45 (br s, 1H, NH); MS(EI) m/e 390 [M⁺].

### Preparation Example 3-7

### 6,8-Dichloro-2-(4-chloro-benzyl)-1,1-dioxo-1,4-dihydro-2H-1λ⁶-benzo[1,2,4]thiadiazin-3-one (Intermediate II-7)

(yield, ˜quantitative), pale yellow solid; m.p. 218-220° C.; ¹H NMR (200 MHz, CDC₃) δ 5.05 (s, 2H, NCH₂Ar), 6.92 (d, J=1.6 Hz, 1H, ArH), 7.23-7.47 (m, 5H, ArH), 9.58 (brs, 1H, NH); MS(EI) m/e 390 [M⁺].

### Preparation Example 3-8

### 2-(2-Bromo-benzyl)-6,8-dichloro-1,1-dioxo-1,4-dihydro-2H-1λ⁶-benzo[1,2,4]thiadiazin-3-one (Intermediate II-8)

(yield, 97%), pale gray solid; m.p. 219-221° C.; ¹H NMR (200 MHz, CDCl₃+CD₃OD) δ 5.18 (s, 2H, NCH₂Ar), 7.09-7.17 (m, 2H, ArH), 7.25-7.33 (m, 3H, ArH), 7.55 (m, 1H, ArH); MS(EI) m/e 436 [M⁺+2], 434 [M⁺].

### Preparation Example 3-9

### 2-(3-Bromo-benzyl)-6,8-dichloro-1,1-dioxo-1,4-dihydro-2H-1λ⁶-benzo[1,2,4]thiadiazin-3-one (Intermediate II-9)

(yield, 90%), white solid; m.p. 189-191° C.; ¹H NMR (200 MHz, CDCl₃+CD₃OD) δ 4.89 (s, 2H, NCH₂Ar), 6.97 (d, J=1.6 Hz, 1H, ArH), 7.08 (m, 1H, ArH), 7.11 (d, J=1.6 Hz, 1H, ArH), 7.24-7.32 (m, 2H, ArH), 7.50 (m, 1H, ArH); MS(EI) m/e 436 [M⁺+2], 434 [M⁺].

### Preparation Example 3-10

### 2-(4-Bromo-benzyl)-6,8-dichloro-1,1-dioxo-1,4-dihydro-2H-1λ⁶-benzo[1,2,4]thiadiazin-3-one (Intermediate II-10)

(yield, 93%), pale gray solid; m.p. 230-232° C.; ¹H NMR (200 MHz, CDCl₃+CD₃OD) δ 5.00 (s, 2H, NCH₂Ar), 7.07 (d, J=1.6 Hz, 1H, ArH), 7.22 (d, J=1.6 Hz, 1H, ArH), 7.36-7.47 (m, 4H, ArH); MS(EI) m/e 436 [M⁺+2].

### Preparation Example 3-11

### 6,8-Dichloro-2-(3-iodo-benzyl)-1,1-dioxo-1,4-dihydro-2H-1λ⁶-benzo[1,2,4]thiadiazin-3-one (Intermediate II-11)

(yield, 95%), white solid; m.p. 183-184° C.; ¹H NMR (200 MHz, CDC₃) δ 5.01 (s, 2H, NCH₂Ar), 6.96 (d, J=1.4 Hz, 1H, ArH), 7.03 (dd, J=7.8, 8.0 Hz, 1H, ArH), 7.27 (d, J=1.4 Hz, 1H, ArH), 7.46 (m, 1H, ArH), 7.63 (m, 1H, ArH), 7.83 (m, 1H, ArH); MS(EI) m/e 481 [M⁺].

### Preparation Example 3-12

### 6,8-Dichloro-2-(4-iodo-benzyl)-1,1-dioxo-1,4-dihydro-2H-1λ⁶-benzo[1,2,4]thiadiazin-3-one (Intermediate II-12)

(yield, 91%), pale gray solid; m.p. 199-200° C.; ¹H NMR (200 MHz, CDCl₃+CD₃OD) δ 4.99 (s, 2H, NCH₂Ar), 7.02-7.08 (m, 2H, ArH), 7.22-7.26 (m, 2H, ArH), 7.63-7.67 (m, 2H, ArH); MS(EI) m/e 481 [M⁺].

### Preparation Example 3-13

### 6,8-Dichloro-2-(2-methyl-benzyl)-1,1-dioxo-1,4-dihydro-2H-1λ⁶-benzo[1,2,4]thiadiazin-3-one (Intermediate II-13)

(yield, 87%), pale yellow solid; m.p. 225-228° C.; ¹H NMR (200 MHz, CDCl₃) δ 2.46 (s, 3H, CH₃), 5.11 (s, 2H, NCH₂Ar), 6.87 (d, J=1.6 Hz, 1H, ArH), 7.15-7.17 (m, 3H, ArH), 7.26-7.30 (m, 2H, ArH); MS(EI) m/e 370 [M⁺], 105.

### Preparation Example 3-14

### 6,8-Dichloro-2-(3-methyl-benzyl)-1,1-dioxo-1,4-dihydro-2H-1λ⁶-benzo[1,2,4]thiadiazin-3-one (Intermediate II-14)

(yield, 78%), white solid; m.p. 164-167° C.; ¹H NMR (200 MHz, CDC₃) δ 2.33 (s, 3H, CH₃), 5.06 (s, 2H, NCH₂Ar), 6.93 (d, J=1.6 Hz, 1H, ArH), 7.09-7.12 (m, 1H, ArH), 7.19-7.29 (m, 4H, ArH), 9.60 (br s, 1H, NH); MS(EI) m/e 348 [M⁺], 105.

### Preparation Example 3-15

### 6,8-Dichloro-2-(4-methyl-benzyl)-1,1-dioxo-1,4-dihydro-2H-1λ⁶-benzo[1,2,4]thiadiazin-3-one (Intermediate II-15)

(yield, 95%), pale light yellow solid; m.p. 192-198° C.; ¹H NMR (200 MHz, CDCl₃) δ 2.33 (s, 3H, CH₃), 5.08 (s, 2H, NCH₂Ar), 6.96 (d, J=1.6 Hz, 1H, ArH), 7.13-7.18 (m, 2H, ArH), 7.28 (d, J=1.6 Hz, 1H, ArH), 7.39-7.43 (m, 2H, ArH); MS(EI) m/e 370 [M⁺], 291.

### Preparation Example 3-16

### 6,8-Dichloro-2-(2-methoxy-benzyl)-1,1-dioxo-1,4-dihydro-2H-1λ⁶-benzo[1,2,4]thiadiazin-3-one (Intermediate II-16)

(yield, 71%), gray solid; m.p. 194-195° C.; ¹H NMR (200 MHz, CDC₃) δ 3.88 (s, 3H, OCH₃), 5.21 (s, 2H, NCH₂Ar), 6.88-6.97 (m, 2H, ArH), 7.25-7.32 (m, 3H, ArH), 9.69 (br s, 1H, NH); MS(EI) m/e 386 [M⁺].

### Preparation Example 3-17

### 6,8-Dichloro-2-(3-methoxy-benzyl)-1,1-dioxo-1,4-dihydro-2H-1λ⁶-benzo[1,2,4]thiadiazin-3-one (Intermediate II-17)

(yield, 78%), shiny white solid; m.p. 151-152° C.; ¹H NMR (200 MHz, CDC₃) δ 3.78 (s, 3H, OCH₃), 5.07 (s, 2H, NCH₂Ar), 6.81-6.86 (m, 1H, ArH), 6.93 (d, J=1.6 Hz, 1H, ArH), 7.05-7.09 (m, 2H, ArH), 7.22-7.29 (m, 2H, ArH), 9.53 (br s, 1H, NH); MS(EI) m/e 386 [M⁺].

### Preparation Example 3-18

### 6,8-Dichloro-2-(4-methoxy-benzyl)-1,1-dioxo-1,4-dihydro-2H-1λ⁶-benzo[1,2,4]thiadiazin-3-one (Intermediate II-18)

(yield, 71%), gray solid; ¹H NMR (200 MHz, CDCl₃) δ 3.77 (s, 3H, OCH₃), 5.03 (s, 2H, NCH₂Ar), 6.83-6.88 (m, 2H, ArH), 6.98 (d, J=2.0 Hz, 1H, ArH), 7.25 (d, J=2.0 Hz, 1H, ArH), 7.42-7.47 (m, 2H, ArH), 9.79 (br s, 1H, NH); MS(EI) m/e 386 [M⁺].

### Preparation Example 3-19

### 6,8-Dichloro-2-(2-nitro-benzyl)-1,1-dioxo-1,4-dihydro-2H-1λ⁶-benzo[1,2,4]thiadiazin-3-one (Intermediate II-19)

(yield, ˜quantitative), white solid; m.p. 230-232° C.; ¹H NMR (200 MHz, CDCl₃+CD₃OD) δ 5.50 (s, 2H, NCH₂Ar), 7.15 (d, J=2.0 Hz, 1H, ArH), 7.26 (d, J=2.0 Hz, 1H, ArH), 7.43-7.66 (m, 3H, ArH), 8.06-8.11 (m, 4H, ArH); MS(EI) m/e 401 [M⁺].

### Preparation Example 3-20

### 6,8-Dichloro-2-(3-nitro-benzyl)-1,1-dioxo-1,4-dihydro-2H-1λ⁶-benzo[1,2,4]thiadiazin-3-one (Intermediate II-20)

(yield, 91%), yellow solid; m.p. 210-217° C.; ¹H NMR (200 MHz, CDC₃) δ 5.14 (s, 2H, NCH₂Ar), 7.06 (d, J=2.0 Hz, 1H, ArH), 7.24 (d, J=1.6 Hz, 1H, ArH), 7.49-7.57 (dd, J=8.2, 7.6 Hz, 1H, ArH), 7.83 (m, 1H, ArH), 8.15 (m, 1H, ArH), 8.36 (m, 1H, ArH); MS(EI) m/e 401[M⁺].

### Preparation Example 3-21

### 6,8-Dichloro-2-(4-nitro-benzyl)-1,1-dioxo-1,4-dihydro-2H-1λ⁶-benzo[1,2,4]thiadiazin-3-one (Intermediate II-21)

(yield, 93%), pale yellow solid; m.p. 222-224° C.; ¹H NMR (200 MHz, CDCl₃+CD₃OD) δ 5.09 (s, 2H, CH₂Ar), 7.04 (d, J=1.6 Hz, 1H, ArH), 7.20 (d, J=1.6 Hz, 1H, ArH), 7.59-7.64 (m, 2H, ArH), 8.11-8.17 (m, 2H, ArH); MS(EI) m/e 401[M⁺].

### Preparation Example 3-22

### 4-(6,8-Dichloro-1,1,3-trioxo-3,4-dihydro-1H-1λ⁶-benzo[1,2,4]thiadiazin-2-ylmethyl)-benzoic acid methyl ester (Intermediate II-22)

(yield, 98%), pale yellow solid; mp 228-231° C.; ¹H NMR (200 MHz, CDC₃) δ 3.90 (s, 3H, OCH₃), 5.12 (s, 2H, NCH₂Ar), 6.91 (d, J=1.6 Hz, 1H, ArH), 7.28 (d, J=1.6 Hz, 1H, ArH), 7.53-7.57 (m, 2H, ArH), 7.99-8.03 (m, 2H, ArH), 8.75 (br s, 1H, NH); MS(EI) m/e 414 [M⁺].

### Preparation Example 3-23

### 6,8-Dichloro-2-(4-cyano-benzyl)-1,1-dioxo-1,4-dihydro-2H-1λ⁶-benzo[1,2,4]thiadiazin-3-one (Intermediate II-23)

(yield, 89%), white solid; m.p. 217-218° C.; ¹H NMR (200 MHz, CDCl₃+CD₃OD) δ 5.08 (s, 2H, NCH₂Ar), 7.07 (d, J=2.0 Hz, 1H, ArH), 7.23 (d, J=2.0 Hz, 1H, ArH), 7.57-7.66 (m, 4H, ArH); MS(EI) m/e 381 [M⁺], 317, 214.

### Preparation Example 3-24

### 6,8-Dichloro-1,1-dioxo-2-[(R)-1-phenyl-ethyl]-1,4-dihydro-2H-1λ⁶-benzo[1,2,4]thiadiazin-3-one (Intermediate II-24)

(yield, 87%), white solid; ¹H NMR (200 MHz, CDC₃) δ 2.03 (d, J=6.8 Hz, 3H, CH₃), 5.84 (q, J=6.8 Hz, 1H, NCHMeAr), 6.64 (d, J=2.0 Hz, 1H, ArH), 7.24-7.39 (m, 4H, ArH), 7.47-7.51 (m, 2H, ArH), 10.37 (br s, 1H, NH); MS(EI) m/e 370 [M⁺].

### Preparation Example 3-25

### 6,8-Dichloro-1,1-dioxo-2-[(S)-1-phenyl-ethyl]-1,4-dihydro-2H-1λ⁶-benzo[1,2,4]thiadiazin-3-one (Intermediate II-25)

(yield, 84%), white solid; ¹H NMR (200 MHz, CDCl₃) δ 2.06 (d, J=7.4 Hz, 3H, CH₃), 5.89 (q, J=7.1 Hz, 1H, NCHMeAr), 6.66 (d, J=1.8 Hz, 1H, ArH), 7.27-7.54 (m, 6H, ArH), 10.20 (br s, 1H, NH); MS(EI) m/e 370 [M⁺].

### Preparation Example 3-26

### 6,8-Dichloro-1,1-dioxo-2-phenyl-1,4-dihydro-2H-1λ⁶-benzo[1,2,4]thiadiazin-3-one (Intermediate II-26)

(yield, 77%), pale white solid; m.p. 217-219° C.; ¹H NMR (200 MHz, CDCl₃) δ 6.93 (d, J=1.6 Hz, 1H, ArH), 7.28 (d, J=1.6 Hz, 1H, ArH), 7.43-7.57 (m, 5H, ArH), 9.33 (br s, 1H, NH); MS(EI) m/e 342 [M⁺].

### Preparation Example 3-27

### 6,8-Dichloro-2-(2-methoxy-phenyl)-1,1-dioxo-1,4-dihydro-2H-1λ⁶-benzo[1,2,4]thiadiazin-3-one (Intermediate II-27)

(yield, 82%), dark violet solid; ¹H NMR (200 MHz, CDCl₃) δ 3.80 (s, 3H, OCH₃), 7.02-7.09 (m, 2H, ArH), 7.13 (d, J=2.0 Hz, 1H, ArH), 7.24 (d, J=2.0 Hz, 1H, ArH), 7.43-7.52 (m, 2H, ArH); MS(EI) m/e 372 [M⁺].

### Preparation Example 3-28

### 6,8-Dichloro-2-(3-methoxy-phenyl)-1,1-dioxo-1,4-dihydro-2H-1λ⁶-benzo[1,2,4]thiadiazin-3-one (Intermediate II-28)

(yield, 95%), pale yellow solid; m.p. 238-240° C.; ¹H NMR (200 MHz, CDCl₃) δ 3.83 (s, 3H, OCH₃), 6.96 (d, J=2.0 Hz, 1H, ArH), 7.01-7.09 (m, 2H, ArH), 7.14 (d, J=2.0 Hz, 1H, ArH), 7.33-7.45 (m, 2H, ArH); MS(EI) m/e 372 [M⁺], 238.

### Preparation Example 3-29

### 6,8-Dichloro-2-(4-methoxy-phenyl)-1,1-dioxo-1,4-dihydro-2H-1λ⁶-benzo[1,2,4]thiadiazin-3-one (Intermediate II-29)

(yield, 85%), gray solid; m.p. 244-246° C.; ¹H NMR (200 MHz, CDCl₃) δ 3.80 (s, 3H, OCH₃), 6.94 (m, 2H, ArH), 7.06 (d, J=1.8 Hz, 1H, ArH), 7.20 (d, J=1.8 Hz, 1H, ArH), 7.39 (m, 2H, ArH); MS(EI) m/e 372 [M⁺].

### Preparation Example 3-30

### 6,8-Dichloro-2-methyl-1,1-dioxo-1,4-dihydro-2H-1λ⁶ benzo[1,2,4]thiadiazin-3-one (Intermediate II-30)

(yield, 78%), white solid; ¹H NMR (200 MHz, CDC₃) δ 3.36 (s, 3H, NCH₃), 7.11 (d, J=2.0 Hz, 1H, ArH), 7.24 (d, J=2.2 Hz, 1H, ArH); MS(EI) m/e 279 [M⁺].

### Preparation Example 3-31

### 6,8-Dichloro-2-ethyl-1,1-dioxo-1,4-dihydro-2H-1λ⁶-benzo[1,2,4]thiadiazin-3-one (Intermediate II-31)

(yield, 92%), pale light yellow solid; m.p. 179-181° C.; ¹H NMR (200 MHz, CDCl₃+CD₃OD) δ 1.37 (t, J=7.0 Hz, 3H, CH₃), 3.96 (q, J=7.0 Hz, 2H, NCH₂), 7.06 (d, J=1.8 Hz, 1H, ArH), 7.22 (d, J=1.8 Hz, 1H, ArH), 10.45 (br s, 1H, NH); MS(EI) m/e 379 [M⁺], 266, 250.

### Preparation Example 3-32

### 6,8-Dichloro-2-propyl-1,1-dioxo-1,4-dihydro-2H-1λ⁶ benzo[1,2,4]thiadiazin-3-one (Intermediate II-32)

(yield, 89%), pale light yellow solid; m.p. 128-131° C.; ¹H NMR (200 MHz, CDCl₃) δ 0.94 (t, J=7.2 Hz, 3H, CH₃), 1.86 (m, 2H, CH₂), 3.90 (t, J=7.4 Hz, 2H, NCH₂), 7.07 (d, J=2.0 Hz, 1H, ArH), 7.27 (d, J=2.0 Hz, 1H, ArH), 10.45 (br s, 1H, NH); MS(EI) m/e 308 [M⁺], 269.

### Preparation Example 3-33

### 6,8-Dichloro-2-butyl-1,1-dioxo-1,4-dihydro-2H-1λ⁶-benzo[1,2,4]thiadiazin-3-one (Intermediate II-33)

(yield, 86%), white solid; m.p. 129-130° C.; ¹H NMR (200 MHz, CDC₃) δ 1.01 (t, J=7.1 Hz, 3H, CH₃), 1.48 (m, 2H, CH₂), 1.82 (m, 2H, CH₂), 4.00 (t, J=7.6 Hz, 2H, NCH₂), 7.07 (d, J=2.0 Hz, 1H, ArH), 7.28 (d, J=1.6 Hz, 1H, ArH), 10.2 (br s, 1H, NH); MS(EI) m/e 322 [M⁺], 308, 284.

### Preparation Example 3-34

### 6,8-Dichloro-2-cyclohexylmethyl-1,1-dioxo-1,4-dihydro-2H-1λ⁶-benzo[1,2,4]thiadiazin-3-one (Intermediate II-34)

(yield, 98%), pale light yellow solid; m.p. 183-185° C.; ¹H NMR (200 MHz, CDCl₃+CD OD) δ 0.93-1.22 (m, 11H, CH₃×3 of cyclohexyl), 1.67-1.76 (m, 5H, CH₂×2 and CH of cyclohexyl), 3.70 (d, J=7.2 Hz, 2H, NCH₂), 7.02 (d, J=1.6 Hz, 1H, ArH), 7.18 (d, J=1.6 Hz, 1H, ArH); MS(EI) m/e 348 [M⁺].

### Preparation Example 3-35

### 6,8-Dichloro-1,1-dioxo-2-phenethyl-1,4-dihydro-2H-1λ⁶-benzo[1,2,4]thiadiazin-3-one (Intermediate II-35)

(yield, 96%), white solid; m.p. 188-189° C.; ¹H NMR (200 MHz, CDC₃) δ 3.12 (t, J=7.9 Hz, 2H, CH₂Ar), 4.21 (t, J=7.9 Hz, 2H, NCH₂), 7.05 (d, J=2.0 Hz, 1H, ArH), 7.19-7.38 (m, 6H, ArH), 10.02 (br s, 1H, NH); MS(EI) m/e 370 [M⁺].

### Preparation Example 3-36

### 6,8-Dichloro-2-[3-(3,5-dimethyl-phenyl)-propyl]-1,1-dioxo-1,4-dihydro-2H-1λ⁶-benzo[1,2,4]thiadiazin-3-one (Intermediate II-36)

(yield, 87%), white solid; ¹H NMR (200 MHz, CDC₃) δ 2.11-2.18 (m, 2H, CH₂), 2.24 (s, 6H, CH₃×2), 2.67 (t, J=7.8 Hz, 2H, CH₂Ar), 4.03 (m, 2H, NCH₂), 6.75 (d, J=2.8 Hz, 1H, ArH), 6.80 (d, J=2.8 Hz, 2H, ArH), 6.98 (d, J=2.2 Hz, 1H, ArH), 7.24 (d, J=2.2 Hz, 1H, ArH), 10.11 (br s, 1H, NH); MS(EI) m/e 412 [M⁺].

### Preparation Example 3-37

### 6,8-Dichloro-2-octyl-1,1-dioxo-1,4-dihydro-2H-1λ⁶-benzo[1,2,4]thiadiazin-3-one (Intermediate II-37)

(yield, 99%), pale light yellow solid; m.p. 98-100° C.; ¹H NMR (200 MHz, CDCl₃) δ 0.86 (t, J=6.8 Hz, 3H, CH₃), 1.28-1.41 (m, 10H, CH₃×5), 1.76-1.87 (m, 2H, CH₂), 3.94 (t, J=7.6 Hz, 2H, NCH₂), 7.04 (d, J=2.0 Hz, 1H, ArH), 7.28 (d, J=2.0 Hz, 1H, ArH), 10.01 (s, 1H, NH); MS(EI) m/e 379 [M⁺], 267, 250.

### Preparation Example 3-38

### 6,8-Dichloro-2-decyl-1,1-dioxo-1,4-dihydro-2H-1λ⁶-benzo[1,2,4]thiadiazin-3-one (Intermediate II-38)

(yield, 77%), dark yellow solid; m.p. 106-107° C.; ¹H NMR (200 MHz, CDCl₃) δ 0.83 (t, J=7.0 Hz, 3H, CH₃1), 1.25-1.83 (m, 16H, CH₂×8), 3.90 (t, J=7.8 Hz, 2H, NCH₂), 6.99 (d, J=2.0 Hz, 1H, ArH), 7.25 (d, J=2.0 Hz, 1H, ArH); MS(EI) m/e 371 [M⁺−Cl].

### Preparation Example 3-39

### 6,8-Dichloro-2-naphthalen-1-ylmethyl-1,1-dioxo-1,4-dihydro-2H-1λ⁶-benzo[1,2,4]thia diazin-3-one (Intermediate II-39)

(yield, 97%), white solid; m.p. 230-232° C.; ¹H NMR (200 MHz, CDCl₃+CD₃OD) δ 5.53 (s, 2H, NCH₂Ar), 7.03 (d, J=2.0 Hz, 1H, ArH), 7.17 (d, J=2.0 Hz, 1H, ArH), 7.30-7.53 (m, 4H, ArH), 7.69-7.82 (m, 2H, ArH), 8.09 (m, 1H, ArH); MS(EI) m/e 406 [M⁺].

### Preparation Example 3-40

### 6,8-Dichloro-1,1-dioxo-2-pyridin-4-ylmethyl-1,4-dihydro-2H-1λ⁶-benzo[1,2,4]thiadiazin-3-one (Intermediate II-40)

(yield, 96%), pale gray solid; m.p. 244-245° C.; ¹H NMR (200 MHz, CDCl₃+CD_{OD) δ} 5.06 (s, 2H, NCH₂Py), 7.13 (d, J=2.0 Hz, 1H, ArH), 7.25 (d, J=2.0 Hz, 1H, ArH), 7.34-7.44 (m, 2H, ArH), 8.48-8.51 (m, 2H, ArH); MS(EI) m/e 357 [M⁺].

### Preparation Example 3-41

### 6,8-Dichloro-2-(5-methyl-furan-2-ylmethyl)-1,1-dioxo-1,4-dihydro-2H-1λ⁶-benzo[1,2,4]thiadiazin-3-one (Intermediate II-41)

(yield, 83%), pale yellow solid; m.p. 112-114° C.; ¹H NMR (200 MHz, CDC₃) δ 2.12 (s, 3H, CH₃ of furanyl), 5.07 (s, 2H, NCH₂Ar), 5.90 (d, J=3.2 Hz, 1H, CH of furanyl ring), 6.33 (d, J=3.2 Hz, 1H, CH of furanyl ring), 7.05 (d, J=2.0 Hz, 1H, ArH), 7.25 (d, J=2.0 Hz, 1H, ArH), 10.23 (br s, 1H, NH); MS(EI) m/e 360 [M⁺].

### Preparation Example 4

### General Procedure for the synthesis of 2,4-Substituted-1,1-dioxo-1,4-dihydro-2H-1λ⁶-benzo[1,2,4]thiadiazin-3-ones (Intermediate III)

To a solution of the appropriate intermediate II (1.0 mmol) in DMF (15 mL) was added a suitable iodides or bromides (1.2 mmol) and a base such as K₂CO₃ (3.0 mmol) or sodium hydride (1.3 mmol). The resulting mixture was allowed to stirred at 80-100° C. for 3 hours. The solvent was evaporated under reduced pressure. The residue was dissolved with ethyl acetate, and washed with 0.5M HCl solution, water and brine. The organic phase was dried over anhydrous MgSO₄ and concentrated in vacuo. The crude was purified by a flash column chromatography (eluent; a mixture of n-hexane and ethyl acetate) to give the corresponding 2,4-substituted-benzo[1,2,4]thiadiazin-3-ones (Intermediate III).

### Preparation Example 4-1

### 2,4-Dibenzyl-6,8-dichloro-1,1-dioxo-1,4-dihydro-2H-1λ⁶-benzo[1,2,4]thiadiazin-3-one (Intermediate III-1)

(yield, 92%), white solid; ¹H NMR (200 MHz, CDC₃) δ 5.17 (s, 2H, NCH₂Ar), 5.28 (s, 2H, NCH₂Ar), 7.02-7.04 (m, 1H, ArH), 7.15-7.40 (m, 9H, ArH), 7.51-7.55 (m, 2H, ArH); MS(EI) m/e 446 [M⁺].

### Preparation Example 4-2

### 4-Benzyl-6,8-dichloro-2-(2-fluoro-benzyl)-1,1-dioxo-1,4-dihydro-2H-1λ⁶-benzo[1,2,4]thiadiazin-3-one (Intermediate III-2)

(yield, 85%), pale gray solid; m.p. 123-124° C.; ¹H NMR (200 MHz, CDC₃) δ 5.23 (s, 2H, NCH₂Ar), 5.25 (s, 2H, NCH₂Ar), 6.99-7.49 (m, 11H, ArH); MS(EI) m/e 464 [M⁺].

### Preparation Example 4-3

### 4-Benzyl-6,8-dichloro-2-(3-fluoro-benzyl)-1,1-dioxo-1,4-dihydro-2H-1λ⁶-benzo[1,2,4]thiadiazin-3-one (Intermediate III-3)

(yield, 83%), white solid; m.p. 119-121° C.; ¹H NMR (200 MHz, CDC₃) δ 5.11 (s, 2H, NCH₂Ar), 5.25 (s, 2H, NCH₂Ar), 6.96-7.04 (m, 2H, ArH), 7.13-7.36 (m, 9H, ArH); MS(EI) m/e 464 [M⁺].

### Preparation Example 4-4

### 4-Benzyl-6,8-dichloro-2-(4-fluoro-benzyl)-1,1-dioxo-1,4-dihydro-2H-1λ⁶-benzo[1,2,4]thiadiazin-3-one (Intermediate III-4)

(yield, 90%), pale gray solid; m.p. 106-107° C.; ¹H NMR (200 MHz, CDCl₃) δ 5.09 (s, 2H, NCH₂Ar), 5.25 (s, 2H, NCH₂Ar), 6.98-7.06 (m, 3H, ArH), 7.11-7.15 (m, 2H, ArH), 7.22-7.37 (m, 4H, ArH), 7.46-7.53 (m, 2H, ArH); MS(EI) m/e 464 [M⁺].

### Preparation Example 4-5

### 4-Benzyl-6,8-dichloro-2-(2-chloro-benzyl)-1,1-dioxo-1,4-dihydro-2H-1λ⁶-benzo[1,2,4]thiadiazin-3-one (Intermediate III-5)

(yield, 96%), white solid; m.p. 84-85° C.; ¹H NMR (200 MHz, CDC₃) δ 5.26 (s, 2H, NCH₂Ar), 5.28 (s, 2H, NCH₂Ar), 7.06 (d, J=1.6 Hz, 1H, ArH), 7.14-7.37 (m, 10H, ArH); MS(EI) m/e 480 [M⁺].

### Preparation Example 4-6

### 4-Benzyl-6,8-dichloro-2-(3-chloro-benzyl)-1,1-dioxo-1,4-dihydro-2H-1λ⁶-benzo[1,2,4]thiadiazin-3-one (Intermediate III-6)

(yield, 83%), pale gray solid; m.p. 144-145° C.; ¹H NMR (200 MHz, CDC₃) δ 5.09 (s, 2H, NCH₂Ar), 5.25 (s, 2H, NCH₂Ar), 7.01 (d, J=2.0 Hz, 1H, ArH), 7.13-7.17 (m, 2H, ArH), 7.23-7.48 (m, 8H, ArH); MS(EI) m/e 480 [M⁺].

### Preparation Example 4-7

### 4-Benzyl-6,8-dichloro-2-(4-chloro-benzyl)-1,1-dioxo-1,4-dihydro-2H-1λ⁶-benzo[1,2,4]thiadiazin-3-one (Intermediate III-7)

(yield, 92%), white solid; m.p. 173-174° C.; ¹H NMR (200 MHz, CDC₃) δ 5.08 (s, 2H, NCH₂Ar), 5.24 (s, 2H, NCH₂Ar), 7.00 (d, J=1.6 Hz, 1H, ArH), 7.11-7.15 (m, 2H, ArH), 7.22-7.38 (m, 6H, ArH), 7.42-7.46 (m, 2H, ArH); MS(EI) m/e 480 [M⁺].

### Preparation Example 4-8

### 4-Benzyl-2-(2-bromo-benzyl)-6,8-dichloro-1,1-dioxo-1,4-dihydro-2H-1λ⁶-benzo[1,2,4]thiadiazin-3-one (Intermediate III-8)

(yield, 88%), pale gray solid; m.p. 105-107° C.; ¹H NMR (200 MHz, CDCl₃) δ 5.25 (s, 4H, NCH₂Ar×2), 7.06 (d, J=2.2 Hz, 1H, ArH), 7.11-7.36 (m, 9H, ArH), 7.55 (m, 1H, ArH); MS(EI) m/e 445 [M⁺−Br].

### Preparation Example 4-9

### 4-Benzyl-2-(3-bromo-benzyl)-6,8-dichloro-1,1-dioxo-1,4-dihydro-2H-1λ⁶-benzo[1,2,4]thiadiazin-3-one (Intermediate III-9)

(yield, 93%), pale yellow solid; m.p. 169-170° C.; ¹H NMR (200 MHz, CDC₃) δ 5.08 (s, 2H, NCH₂Ar), 5.25 (s, 2H, NCH₂Ar), 7.01 (d, J=1.6 Hz, 1H, ArH), 7.13-7.63 (m, 10H, ArH); MS(EI) m/e 526 [M⁺+2].

### Preparation Example 4-10

### 4-Benzyl-2-(4-bromo-benzyl)-6,8-dichloro-1,1-dioxo-1,4-dihydro-2H-1λ⁶-benzo[1,2,4]thiadiazin-3-one (Intermediate III-10)

(yield, 93%), pale gray solid; m.p. 148-149° C.; ¹H NMR (200 MHz, CDCl₃) δ 5.07 (s, 2H, NCH₂Ar), 5.24 (s, 2H, NCH₂Ar), 7.00 (d, J=1.8 Hz, 1H, ArH), 7.11-7.15 (m, 2H, ArH), 7.23 (d, J=1.8 Hz, 1H, ArH), 7.26-7.48 (m, 7H, ArH); MS(EI) m/e 526 [M⁺+2].

### Preparation Example 4-11

### 4-Benzyl-6,8-dichloro-2-(3-iodo-benzyl)-1,1-dioxo-1,4-dihydro-2H-1λ⁶-benzo[1,2,4]thiadiazin-3-one (Intermediate III-11)

(yield, 65%), white solid; m.p. 147-149° C.; ¹H NMR (200 MHz, CDC₃) δ 5.06 (s, 2H, NCH₂Ar), 5.25 (s, 2H, NCH₂Ar), 7.01-7.16 (m, 4H, ArH), 7.23-7.38 (m, 4H, ArH), 7.46 (m, 1H, ArH), 7.64 (m, 1H, ArH), 7.83 (m, 1H, ArH); MS(EI) m/e 572 [M⁺].

### Preparation Example 4-12

### 4-Benzyl-6,8-dichloro-2-(4-iodo-benzyl)-1,1-dioxo-1,4-dihydro-2H-1λ⁶-benzo[1,2,4]thiadiazin-3-one (Intermediate III-12)

(yield, 88%), pale yellow solid; m.p. 155-158° C.; ¹H NMR (200 MHz, CDCl₃+CD₃OD) δ 5.06 (s, 2H, NCH₂Ar), 5.26 (s, 2H, NCH₂Ar), 7.06 (d, J=1.8 Hz, 1H, ArH), 7.12-7.70 (m, 10H, ArH); MS(EI) m/e 572 [M⁺].

### Preparation Example 4-13

### 4-Benzyl-6,8-dichloro-2-(2-methyl-benzyl)-1,1-dioxo-1,4-dihydro-2H-1λ⁶-benzo[1,2,4]thiadiazin-3-one (Intermediate III-13)

(yield, 84%), white solid; m.p. 149-152° C.; ¹H NMR (200 MHz, CDC₃) δ 2.43 (s, 3H, CH₃), 5.20 (s, 2H, NCH₂Ar), 5.28 (s, 2H, NCH₂Ar), 7.05 (d, J=1.6 Hz, 1H, ArH), 7.13-7.21 (m, 6H, ArH), 7.28-7.34 (m, 4H, ArH); MS(EI) m/e 460 [M⁺], 355, 305.

### Preparation Example 4-14

### 4-Benzyl-6,8-dichloro-2-(3-methyl-benzyl)-1,1-dioxo-1,4-dihydro-2H-1λ⁶-benzo[1,2,4]thiadiazin-3-one (Intermediate III-14)

(yield, 84%), white solid; m.p. 145-148° C.; ¹H NMR (200 MHz, CDCl₃) δ 2.33 (s, 3H, CH₃), 5.10 (s, 2H, NCH₂Ar), 5.25 (s, 2H, NCH₂Ar), 6.99 (d, J=1.6 Hz, 1H, ArH), 7.09-7.18 (m, 4H, ArH), 7.22 (d, J=1.6 Hz, 1H, ArH), 7.26-7.37 (m, 5H, ArH); MS(EI) m/e 460 [M⁺].

### Preparation Example 4-15

### 4-Benzyl-6,8-dichloro-2-(4-methyl-benzyl)-1,1-dioxo-1,4-dihydro-2H-1λ⁶-benzo[1,2,4]thiadiazin-3-one (Intermediate III-15)

(yield, 96%), white solid; m.p.=143-146° C.; ¹H NMR (200 MHz, CDC₃) δ 2.33 (s, 3H, CH₃), 5.10 (s, 2H, NCH₂Ar), 5.24 (s, 2H, NCH₂Ar), 6.99 (d, J=1.6 Hz, 1H, ArH), 7.12-7.16 (m, 2H, ArH), 7.21 (d, J=1.6 Hz, 1H, ArH), 7.26-7.41 (m, 7H, ArH); MS(EI) m/e 460 [M⁺].

### Preparation Example 4-16

### 4-Benzyl-6,8-dichloro-2-(2-methoxy-benzyl)-1,1-dioxo-1,4-dihydro-2H-1λ⁶-benzo[1,2,4]thiadiazin-3-one (Intermediate III-16)

(yield, 85%), shiny white solid; ¹H NMR (200 MHz, CDC₃) δ 3.76 (s, 3H, OCH₃), 5.20 (s, 2H, NCH₂Ar), 5.23 (s, 2H, NCH₂Ar), 6.82-6.94 (m, 2H, ArH), 7.01 (m, 1H, ArH), 7.08-7.12 (m, 2H, ArH), 7.21-7.31 (m, 6H, ArH); MS(EI) m/e 476 [M⁺].

### Preparation Example 4-17

### 4-Benzyl-6,8-dichloro-2-(3-methoxy-benzyl)-1,1-dioxo-1,4-dihydro-2H-1λ⁶-benzo[1,2,4]thiadiazin-3-one (Intermediate III-17)

(yield, 89%), pale gray solid; ¹H NMR (200 MHz, CDCl₃) δ 3.78 (s, 3H, OCH₃), 5.11 (s, 2H, NCH₂Ar), 5.25 (s, 2H, CH₂Ar), 6.85 (m, 1H, ArH), 7.00-7.17 (m, 4H, ArH), 7.21-7.37 (m, 6H, ArH); MS(EI) m/e 478 [M⁺+2].

### Preparation Example 4-18

### 4-Benzyl-6,8-dichloro-2-(4-methoxy-benzyl)-1,1-dioxo-1,4-dihydro-2H-1λ⁶-benzo[1,2,4]thiadiazin-3-one (Intermediate III-18)

(yield, 82%), white solid; ¹H NMR (200 MHz, CDC₃) δ 3.82 (s, 3H, OCH₃), 5.11 (s, 2H, NCH₂Ar), 5.27 (s, 2H, NCH₂Ar), 6.86-6.91 (m, 2H, ArH), 7.01 (d, J=1.6 Hz, 1H, ArH), 7.15-7.19 (m, 2H, ArH), 7.24 (d, J=1.6 Hz, 1H, ArH), 7.29-7.36 (m, 3H, ArH), 7.46-7.50 (m, 2H, ArH); MS(EI) m/e 476 [M⁺].

### Preparation Example 4-19

### 4-Benzyl-6,8-dichloro-2-(2-nitro-benzyl)-1,1-dioxo-1,4-dihydro-2H-1λ⁶-benzo[1,2,4]thiadiazin-3-one (Intermediate III-19)

(yield, 64%), pale yellow solid; m.p. 126-128° C.; ¹H NMR (200 MHz, CDCl₃) δ 5.26 (s, 2H, NCH₂Ar), 5.57 (s, 2H, NCH₂Ar), 7.08 (d, J=1.6 Hz, 1H, ArH), 7.09-7.64 (m, 9H, ArH), 8.10 (m, 1H, ArH); MS(EI) m/e 491 [M⁺].

### Preparation Example 4-20

### 4-Benzyl-6,8-dichloro-2-(3-nitro-benzyl)-1,1-dioxo-1,4-dihydro-2H-1λ⁶-benzo[1,2,4]thiadiazin-3-one (Intermediate III-20)

(yield, 65%), pale yellow solid; mp 117-121° C.; ¹H NMR (200 MHz, CDCl₃) δ 5.24 (s, 2H, NCH₂Ar), 5.29 (s, 2H, NCH₂Ar), 7.07 (d, J=2.0 Hz, 1H, ArH), 7.18 (m, 1H, ArH), 7.21 (d, J=2.0 Hz, 1H, ArH), 7.28-7.38 (m, 4H, ArH), 7.56 (dd, J=7.8, 8.2 Hz, 1H, ArH), 7.86 (m, 1H, ArH), 8.18 (m, 1H, ArH), 8.38 (m, 1H, ArH); MS(EI) m/e 491 [M⁺].

### Preparation Example 4-21

### 4-Benzyl-6,8-dichloro-2-(4-nitro-benzyl)-1,1-dioxo-1,4-dihydro-2H-1λ⁶-benzo[1,2,4]thiadiazin-3-one (Intermediate III-21)

(yield, 64%), pale yellow solid; m.p. 149-150° C.; ¹H NMR (200 MHz, CDCl₃) δ 5.19 (s, 2H, NCH₂Ar), 5.25 (s, 2H, NCH₂Ar), 7.04 (d, J=2.0 Hz, 1H, ArH), 7.13-7.17 (m, 2H, ArH), 7.26-7.37 (m, 4H, ArH), 7.65-7.69 (m, 2H, ArH), 8.18-8.22 (m, 2H, ArH); MS(EI) m/e 491 [M⁺].

### Preparation Example 4-22

### 4-(4-Benzyl-6,8-dichloro-1,1,3-trioxo-3,4-dihydro-1H-1λ⁶-benzo[1,2,4]thiadiazin-2-ylmethyl)-benzoic acid methyl ester (Intermediate III-22)

(yield, 91%), yellow solid; mp 110-113° C.; ¹H NMR (200 MHz, CDCl₃) δ 3.91 (s, 3H, OCH₃), 5.18 (s, 2H, NCH₂Ar), 5.25 (s, 2H, NCH₂Ar), 7.02 (d, J=1.6 Hz, 1H, ArH), 7.13-7.17 (m, 2H, ArH), 7.25 (d, J=1.6 Hz, 1H, ArH), 7.27-7.34 (m, 3H, ArH), 7.53-7.57 (m, 2H, ArH), 7.80-8.04 (m, 2H, ArH); MS(EI) m/e 504 [M⁺].

### Preparation Example 4-23

### 4-(4-Benzyl-6,8-dichloro-1,1,3-trioxo-3,4-dihydro-1H-1λ⁶-benzo[1,2,4]thiadiazin-2-ylmethyl)-benzonitrile (Intermediate III-23)

(yield, 87%), white solid; m.p. 167-168° C.; ¹H NMR (200 MHz, CDC₃) δ 5.17 (s, 2H, NCH₂Ar), 5.27 (s, 2H, NCH₂Ar), 7.06 (d, J=1.8 Hz, 1H, ArH), 7.14-7.18 (m, 2H, ArH), 7.27-7.41 (m, 4H, ArH), 7.58-7.69 (m, 4H, ArH); MS(EI) m/e 471 [M⁺].

### Preparation Example 4-24

### 4-Benzyl-6,8-dichloro-1,1-dioxo-2-[(R)-1-phenyl-ethyl]-1,4-dihydro-2H-1λ⁶-benzo[1,2,4]thiadiazin-3-one (Intermediate III-24)

(yield, 75%), white solid; ¹H NMR (200 MHz, CDC₃) δ 2.06 (d, J=7.2 Hz, 3H, CH₃), 5.04 (d, J=16.8 Hz, 1H, NCHHAr), 5.26 (d, J=16.8 Hz, 1H, NCHHAr), 5.92 (q, J=7.2 Hz, 1H, NCHMeAr), 6.96-7.01 (m, 2H, ArH), 7.24-7.28 (m, 3H, ArH), 7.33-7.39 (m, 5H, ArH), 7.46-7.49 (m, 2H, ArH); MS(EI) m/e 460 [M⁺].

### Preparation Example 4-25

### 4-Benzyl-6,8-dichloro-1,1-dioxo-2-[(S)-1-phenyl-ethyl]-1,4-dihydro-2H-1λ⁶-benzo[1,2,4]thiadiazin-3-one (Intermediate III-25)

(yield, 74%), white solid; ¹H NMR (200 MHz, CDC₃) δ 2.05 (d, J=7.1 Hz, 3H, CH₃), 5.03 (d, J=16.8 Hz, 1H, NCHHAr), 5.25 (d, J=16.8 Hz, 1H, NCHHAr), 5.91 (q, J=7.1 Hz, 1H, NCHMeAr), 6.94-6.99 (m, 3H, ArH), 7.23-7.39 (m, 7H, ArH), 7.44-7.49 (m, 2H, ArH); MS(EI) m/e 460 [M⁺].

### Preparation Example 4-26

### 4-Benzyl-6,8-dichloro-1,1-dioxo-2-phenyl-1,4-dihydro-2H-1λ⁶-benzo[1,2,4]thiadiazin-3-one (Intermediate III-26)

(yield, 62%), white solid; m.p. 161-163° C.; ¹H NMR (200 MHz, CDC₃) δ 5.33 (s, 2H, NCH₂Ar), 7.16 (m, 1H, ArH), 7.24-7.38 (m, 6H, ArH), 7.43-7.56 (m, 5H, ArH); MS(EI) m/e 433 [M⁺+1].

### Preparation Example 4-27

### 4-Benzyl-6,8-dichloro-2-(2-methoxy-phenyl)-1,1-dioxo-1,4-dihydro-2H-1λ⁶-benzo[1,2,4]thiadiazin-3-one (Intermediate III-27)

(yield, 93%), white solid; mp 190-191° C.; ¹H NMR (200 MHz, CDC₃) δ 3.82 (s, 3H, OCH₃), 5.12 (d, J=16.8 Hz, 1H, NCHHAr), 5.43 (d, J=16.8 Hz, 1H, NCHHAr), 7.01-7.12 (m, 3H, ArH), 7.29-7.57 (m, 8H, ArH); MS(EI) m/e 462 [M⁺].

### Preparation Example 4-28

### 4-Benzyl-6,8-dichloro-2-(3-methoxy-phenyl)-1,1-dioxo-1,4-dihydro-2H-1λ⁶-benzo[1,2,4]thiadiazin-3-one (Intermediate III-28)

(yield, 88%), white solid; m.p. 164-167° C.; ¹H NMR (200 MHz, CDC₃) δ 3.84 (s, 3H, OCH₃), 5.32 (s, 2H, NCH₂Ar), 6.98 (d, J=2.0 Hz, 1H, ArH), 7.02 (d, J=2.0 Hz, 1H, ArH), 7.11 (m, 1H, ArH), 7.27-7.49 (m, 7H, ArH); MS(EI) m/e 462 [M⁺].

### Preparation Example 4-29

### 4-Benzyl-6,8-dichloro-2-(4-methoxy-phenyl)-1,1-dioxo-1,4-dihydro-2H-1λ⁶-benzo[1,2,4]thiadiazin-3-one (Intermediate III-29)

(yield, 89%), white solid; ¹H NMR (200 MHz, CDC₃) δ 3.84 (s, 3H, OCH₃), 5.31 (s, 2H, NCH₂Ar), 6.98-7.03 (m, 2H, ArH), 7.12 (d, J=1.8 Hz, 1H, ArH), 7.26-7.39 (m, 8H, ArH); MS(EI) m/e 462 [M⁺].

### Preparation Example 4-30

### 6,8-Dichloro-2,4-dimethyl-1,1-dioxo-1,4-dihydro-2H-1λ⁶-benzo[1,2,4]thiadiazin-3-one (Intermediate III-30)

(yield, 89%), white solid; ¹H NMR (200 MHz, CDC₃) δ 3.39 (s, 3H, NCH₃), 3.41 (s, 3H, NCH₃), 7.18 (d, J=1.8 Hz, 1H, ArH), 7.32 (d, J=1.8 Hz, 1H, ArH); MS(EI) m/e 294 [M⁺].

### Preparation Example 4-31

### 4-Benzyl-6,8-dichloro-2-methyl-1,1-dioxo-1,4-dihydro-2H-1λ⁶-benzo[1,2,4]thiadiazin-3-one (Intermediate III-31)

(yield, 67%), white solid; ¹H NMR (200 MHz, CDC₃) δ 3.42 (s, 3H, NCH₃), 5.25 (s, 2H, NCH₂Ar), 7.03 (d, J=1.6 Hz, 1H, ArH), 7.19-7.39 (m, 6H, ArH); MS(EI) m/e 370 [M⁺].

### Preparation Example 4-32

### 4-Benzyl-6,8-dichloro-2-ethyl-1,1-dioxo-1,4-dihydro-2H-1λ⁶-benzo[1,2,4]thiadiazin-3-one (Intermediate III-32)

(yield, 87%), white solid; m.p. 165-167° C.; ¹H NMR (200 MHz, CDC₃) δ 1.41 (t, J=6.8 Hz, 3H, CH₃), 4.02 (q, J=7.0 Hz, 2H, NCH₂), 5.28 (s, 2H, NCH₂Ar), 7.04 (d, J=1.6 Hz, 1H, ArH), 7.22-7.42 (m, 6H, ArH); MS(EI) m/e 364 [M⁺], 248.

### Preparation Example 4-33

### 4-Benzyl-6,8-dichloro-2-propyl-1,1-dioxo-1,4-dihydro-2H-1λ⁶-benzo[1,2,4]thiadiazin-3-one (Intermediate III-33)

(yield, 87%), white solid; m.p. 88-91° C.; ¹H NMR (200 MHz, CDC₃) δ 0.94 (t, J=7.8 Hz, 3H, CH₃), 1.74-1.89 (m, 2H, CH₂), 3.90 (t, J=7.2 Hz, 2H, NCH₂), 5.25 (s, 2H, NCH₂Ar), 7.03 (d, J=1.6 Hz, 1H, ArH), 7.19-7.41 (m, 6H, ArH); MS(EI) m/e 398 [M⁺], 248.

### Preparation Example 4-34

### 4-Benzyl-2-butyl-6,8-dichloro-1,1-dioxo-1,4-dihydro-2H-1λ⁶-benzo[1,2,4]thiadiazin-3-one (Intermediate III-34)

(yield, 87%), white solid; m.p. 110-113° C.; ¹H NMR (200 MHz, CDC₃) δ 0.99 (t, J=7.4 Hz, 3H, CH₃), 1.42 (m, 2H, CH₂), 1.83 (m, 2H, CH₂), 3.99 (t, J=7.4 Hz, 2H, NCH₂), 5.28 (s, 2H, NCH₂Ar), 7.06 (d, J=1.8 Hz, 1H, ArH), 7.22-7.39 (m, 6H, ArH); MS(EI) m/e 412 [M⁺].

### Preparation Example 4-35

### 4-Benzyl-6,8-dichloro-2-cyclohexylmethyl-3,4-dihydro-2H-1λ⁶-benzo[1,2,4]thiadiazine 1,1-dioxide (Intermediate III-35)

(yield, 98%), white solid; m.p. 179-181° C.; ¹H NMR (200 MHz, CDC₃) δ 0.92-1.25 (m, 6H, CH₃×3 of cyclohexyl), 1.69-1.75 (m, 5H, CH₃×2 and CH of cyclohexyl), 3.81 (d, J=7.0 Hz, 2H, NCH₂), 5.25 (s, 2H, NCH₂Ar), 7.02 (d, J=1.6 Hz, 1H, ArH), 7.18-7.39 (m, 6H, ArH); MS(EI) m/e 452 [M⁺].

### Preparation Example 4-36

### 4-Benzyl-6,8-dichloro-1,1-dioxo-2-phenethyl-1,4-dihydro-2H-1λ⁶-benzo[1,2,4]thiadiazin-3-one (Intermediate III-36)

(yield, 96%), white solid; m.p. 89-90° C.; ¹H NMR (200 MHz, CDC₃) δ 3.14 (t, J=7.6 Hz, 2H, CH₂Ar), 4.26 (t, J=7.6 Hz, 2H, NCH₂), 5.25 (s, 2H, NCH₂Ar), 7.03 (d, J=1.8 Hz, 1H, ArH), 7.16-7.44 (m, 11H, ArH); MS(EI) m/e 460 [M⁺].

### Preparation Example 4-37

### 4-Benzyl-6,8-dichloro-2-[3-(3,5-dimethyl-phenyl)-propyl]-1,1-dioxo-1,4-dihydro-2H-1λ₆-benzo[1,2,4]thiadiazin-3-one (Intermediate III-37)

(yield, 76%), white solid; m.p. 152-153° C.; ¹H NMR (200 MHz, CDC₃) δ 2.09-2.22 (m, 2H, CH₂), 2.30 (s, 6H, CH₃×2), 2.68 (t, J=7.8 Hz, 2H, CH₂Ar), 4.08 (m, 2H, NCH₂), 5.29 (s, 2H, NCH₂Ar), 6.84 (m, 3H, ArH), 7.05 (d, J=2.2 Hz, 1H, ArH), 7.23-7.27 (m, 3H, ArH), 7.35-7.40 (m, 3H, ArH); MS(EI) m/e 502 [M⁺], 438, 357.

### Preparation Example 4-38

### 4-Benzyl-6,8-dichloro-2-octyl-1,1-dioxo-1,4-dihydro-2H-1λ⁶-benzo[1,2,4]thiadiazin-3-one (Intermediate III-38)

(yield, 96%), white solid; m.p. 78-81° C.; ¹H NMR (200 MHz, CDC₃) δ 0.86 (t, J=6.4 Hz, 3H, CH₃), 1.28-1.40 (m, 10H, CH₃×5), 1.75-1.86 (m, 2H, CH₂), 3.95 (t, J=7.8 Hz, 2H, NCH₂), 5.28 (s, 2H, NCH₂Ar), 7.05 (d, J=1.6 Hz, 1H, ArH), 7.22-7.43 (m, 6H, ArH); MS(EI) m/e 468 [M⁺].

### Preparation Example 4-39

### 4-Benzyl-6,8-dichloro-2-decyl-1,1-dioxo-1,4-dihydro-2H-1λ⁶-benzo[1,2,4]thiadiazin-3-one (Intermediate III-39)

(yield, 78%), colorless oil; ¹H NMR (200 MHz, CDCl₃) δ 0.84 (t, J=6.6 Hz, 3H, CH of decyl), 1.25-1.33 (m, 14H, CH₂×7 of decyl), 1.80 (m, 2H, CH of decyl), 3.92 (t, J=7.8 Hz, 2H, NCH of decyl), 5.25 (s, 2H, NCH₂Ar), 7.02 (d, J=1.6 Hz, 1H, ArH), 7.19-7.40 (m, 6H, ArH); MS(EI) m/e 496 [M⁺].

### Preparation Example 4-40

### 4-Benzyl-6,8-dichloro-2-naphthalen-1-ylmethyl-1,1-dioxo-1,4-dihydro-2H-1λ⁶-benzo[1,2,4]thiadiazin-3-one (Intermediate III-40)

(yield, 86%), white solid; m.p. 113-114° C.; ¹H NMR (200 MHz, CDC₃) δ 5.26 (s, 2H, NCH₂Ar), 5.68 (s, 2H, NCH₂Ar), 7.00 (d, J=1.6 Hz, 1H, ArH), 7.08-7.12 (m, 2H, ArH), 7.25 (d, J=1.6 Hz, 1H, ArH), 7.28-7.59 (m, 7H, ArH), 7.81-7.91 (m, 2H, ArH), 8.19 (m, 1H, ArH); MS(EI) m/e 496 [M⁺].

### Preparation Example 4-41

### 4-Benzyl-6,8-dichloro-1,1-dioxo-2-pyridin-4-ylmethyl-1,4-dihydro-2H-1λ⁶-benzo[1,2,4]thiadiazin-3-one (Intermediate III-41)

(yield, 44%), brown solid; m.p. 178-180° C.; ¹H NMR (200 MHz, CDC₃) δ 5.11 (s, 2H, NCH₂Ar), 5.26 (s, 2H, NCH₂Ar), 7.05 (d, J=2.0 Hz, 1H, ArH), 7.13-7.17 (m, 2H, ArH), 7.27 (d, J=2.0 Hz, 1H, ArH), 7.26-7.37 (m, 5H, ArH), 8.57-8.60 (m, 2H, ArH); MS(EI) m/e 447 [M⁺].

### Preparation Example 4-42

### 4-Benzyl-6,8-dichloro-2-(5-methyl-furan-2-ylmethyl)-1,1-dioxo-1,4-dihydro-2H-1λ⁶-benzo[1,2,4]thiadiazin-3-one (Intermediate III-42)

(yield, 83%), pale yellow solid: ¹H NMR (200 MHz, CDC₃) δ 2.28 (s, 3H, CH of furanyl), 5.11 (s, 2H, NCH₂Ar), 5.28 (s, 2H, NCH₂Ar), 5.92 (d, J=3.2 Hz, 1H, CH of furanyl), 6.34 (d, J=3.2 Hz, 1H, CH of furanyl), 7.02 (d, J=1.6 Hz, 1H, ArH), 7.20-7.40 (m, 5H, ArH), 7.23 (d, J=1.6 Hz, 1H, ArH); MS(EI) m/e 450 [M⁺].

### EXAMPLES

### General Procedure for the Synthesis of 8-cyclic amine substituted-1,1-dioxo-1,4-dihydro-2H-1λ⁶-benzo[1,2,4]thiadiazin-3-ones

To a solution of the appropriate intermediate III (1.0 mmol) in MeCN (30□) was added the proper cyclic amines (3.0 mmol) and K₂CO₃ (3.0 mmol). The resulting mixture was heated at ambient temperature. After the starting intermediate III was consumed up, the solvent was evaporated under reduced pressure. The residue was dissolved with ethyl acetate, and washed with 0.5 M HCl solution, water and brine. The organic phase was dried over anhydrous MgSO₄ and concentrated in vacuo. The crude was purified by a flash column chromatography (eluent; a mixture of a mixture of methylene chloride and methanol) to afford the corresponding 8-cyclic amine substituted-benzo[1,2,4]thiadiazin-3-ones (Examples).

### Example 1

### 2,4-Dibenzyl-6-chloro-8-(4-methyl-piperazin-1-yl)-1,1-dioxo-1,4-dihydro-2H-1λ⁶-benzo[1,2,4]thiadiazin-3-one

(yield, 55%), white solid; m.p. 100-101° C.; ¹H NMR (200 MHz, CDC₃) δ 2.41 (s, 3H, NCH₃), 2.69-2.72 (m, 4H, NCH₂×2), 3.16-3.18 (m, 4H, NCH₂×2), 5.13 (s, 2H, NCH₂Ar), 5.24 (s, 2H, NCH₂Ar), 6.80 (s, 1H, ArH), 6.90 (s, 1H, ArH), 7.16-7.20 (m, 2H, ArH), 7.26-7.38 (m, 6H, ArH), 7.47-7.51 (m, 2H, ArH); MS(EI) m/e 509 [M⁺−1]; HRMS m/e Cacld. for C₂₆H₂₇N₄O₃S₁Cl₁510.1492, found 510.1473.

### Example 2

### 4-Benzyl-6-chloro-2-(2-fluoro-benzyl)-8-(4-methyl-piperazin-1-yl)-1,1-dioxo-1,4-dihydro-2H-1λ⁶-benzo[1,2,4]thiadiazin-3-one

(yield, 64%), white solids; m.p. 118-120° C.; ¹H NMR (200 MHz, CDC₃) δ 2.35 (s, 3H, NCH₃), 2.61-2.63 (m, 4H, NCH₂×2), 3.10-3.13 (m, 4H, NCH₂×2), 5.20 (s, 2H, NCH₂Ar), 5.23 (s, 2H, NCH₂Ar), 6.80 (d, J=1.6 Hz, 1H, ArH), 6.87 (d, J=1.6 Hz, 1H, ArH), 6.98-7.43 (m, 9H, ArH); MS(EI) m/e 528 [M⁺]; HRMS m/e Cacld. for C₂₆H₂₆N₄O₃F₁S₁Cl₁ 528.1398, found 528.1400.

### Example 3

### 4-Benzyl-6-chloro-2-(3-fluoro-benzyl)-8-(4-methyl-piperazin-1-yl)-1,1-dioxo-1,4-dihydro-2H-1λ⁶-benzo[1,2,4]thiadiazin-3-one

(yield, 64%), white solid; m.p. 85-87° C.; ¹H NMR (200 MHz, CDC₃) δ 2.37 (s, 3H, NCH₃), 2.62-2.66 (m, 4H, NCH₂×2), 3.12-3.16 (m, 4H, NCH₂×2), 5.07 (s, 2H, NCH₂Ar), 5.21 (s, 2H, NCH₂Ar), 6.79 (d, J=1.6 Hz, 1H, ArH), 6.88 (d, J=1.6 Hz, 1H, ArH), 6.93-7.02 (m, 2H, ArH), 7.15-7.36 (m, 8H, ArH); MS(EI) m/e 528 [M⁺]; HRMS m/e Cacld. for C₂₆H₂₆N₄O₃F₁S₁Cl₁ 528.1398, found 528.1424.

### Example 4

### 4-Benzyl-6-chloro-2-(4-fluoro-benzyl)-8-(4-methyl-piperazin-1-yl)-1,1-dioxo-1,4-dihydro-2H-1λ⁶-benzo[1,2,4]thiadiazin-3-one

(yield, 66%), white solid; m.p. 112-113° C.; ¹H NMR (200 MHz, CDC₃) δ 2.39 (s, 3H, NCH₃), 2.66-2.70 (m, 4H, NCH₂×2), 3.14-3.19 (m, 4H, NCH₂×2), 5.07 (s, 2H, NCH₂Ar), 5.23 (s, 2H, NCH₂Ar), 6.79 (d, J=1.6 Hz, 1H, ArH), 6.89 (d, J=1.6 Hz, 1H, ArH), 6.98-7.07 (m, 2H, ArH), 7.16-7.20 (m, 2H, ArH), 7.27-7.34 (m, 3H, ArH), 7.47-7.54 (m, 2H, ArH); MS(EI) m/e 528 [M⁺]; HRMS m/e Cacld. for C₂₆H₂₆N₄O₃F₁S₁Cl₁ 528.1398, found 528.1394.

### Example 5

### 4-Benzyl-6-chloro-2-(2-chloro-benzyl)-8-(4-methyl-piperazin-1-yl)-1,1-dioxo-1,4-dihydro-2H-1λ⁶-benzo[1,2,4]thiadiazin-3-one

(yield, 82%), white solid; m.p. 132-133° C.; ¹H NMR (200 MHz, CDC₃) δ 2.39 (s, 3H, NCH₃), 2.65-2.72 (m, 4H, NCH₂×2), 3.14-3.19 (m, 4H, NCH₂×2), 5.25-5.27 (m, 4H, NCH₂Ar×2), 6.87 (d, J=1.6 Hz, 1H, ArH), 6.93 (d, J=1.6 Hz, 1H, ArH), 7.21-7.39 (m, 9H, ArH); MS(EI) m/e 544 [M⁺]; HRMS m/e Cacld. for C₂₆H₂₆N₄O₃S₁Cl₂ 544.1103, found 544.1108.

### Example 6

### 4-Benzyl-6-chloro-2-(3-chloro-benzyl)-8-(4-methyl-piperazin-1-yl)-1,1-dioxo-1,4-dihydro-2H-1λ⁶-benzo[1,2,4]thiadiazin-3-one

(yield, 71%), white solid; m.p. 87-88° C.; ¹H NMR (200 MHz, CDC₃) δ 2.40 (s, 3H, NCH₃), 2.65-2.69 (m, 4H, NCH₂×2), 3.15-3.19 (m, 4H, NCH₂×2), 5.09 (s, 2H, NCH₂Ar), 5.24 (s, 2H, NCH₂Ar), 6.81 (d, J=1.6 Hz, 1H, ArH), 6.91 (d, J=1.6 Hz, 1H, ArH), 7.18-7.49 (m, 9H, ArH); MS(EI) m/e 544[M⁺]; HRMS m/e Cacld. for C₂₆H₂₆N₄O₃S₁Cl₂ 544.1103, found 544.1112.

### Example 7

### 4-Benzyl-6-chloro-2-(4-chloro-benzyl)-8-(4-methyl-piperazin-1-yl)-1,1-dioxo-1,4-dihydro-2H-1λ⁶-benzo[1,2,4]thiadiazin-3-one

(yield, 76%), white solid; m.p. 99-100° C.; ¹H NMR (200 MHz, CDC₃) δ 2.39 (s, 3H, NCH₃), 2.66-2.72 (m, 4H, NCH₂×2), 3.13-3.18 (m, 4H, NCH₂×2), 5.05 (s, 2H, NCH₂Ar), 5.21 (s, 2H, NCH₂Ar), 6.78 (d, J=1.6 Hz, 1H, ArH), 6.89 (d, J=2.0 Hz, 1H, ArH), 7.13-7.44 (m, 9H, ArH); MS(EI) m/e 544 [M⁺]; HRMS m/e Cacld. for C₂₆H₂₆N₄O₃S₁Cl₂ 544.1103, found 544.1100.

### Example 8

### 4-Benzyl-2-(2-bromo-benzyl)-6-chloro-8-(4-methyl-piperazin-1-yl)-1,1-dioxo-1,4-dihydro-2H-1λ⁶-benzo[1,2,4]thiadiazin-3-one

(yield, 63%), white solid; m.p. 107-109° C.; ¹H NMR (200 MHz, CDC₃) δ 2.34 (s, 3H, NCH₃), 2.61-2.64 (m, 4H, NCH₂×2), 3.11-3.16 (m, 4H, NCH₂×2), 5.21 (s, 2H, NCH₂Ar), 5.24 (s, 2H, NCH₂Ar), 6.85 (d, J=2.0 Hz, 1H, ArH), 6.91 (d, J=2.0 Hz, 1H, ArH), 7.91-7.33 (m, 8H, ArH), 7.54 (m, 1H, ArH); MS(EI) m/e 590 [M⁺+2]; HRMS m/e Cacld. for C₂₆H₂₆N₄O₃S₁Cl₁Br₁ 588.0597, found 589.9684.

### Example 9

### 4-Benzyl-2-(3-bromo-benzyl)-6-chloro-8-(4-methyl-piperazin-1-yl)-1,1-dioxo-1,4-dihydro-2H-1λ⁶-benzo[1,2,4]thiadiazin-3-one

(yield, 58%), white solid; m.p. 96-98° C.; ¹H NMR (200 MHz, CDC₃) δ 2.37 (s, 3H, NCH₃), 2.64-2.67 (m, 4H, NCH₂×2), 3.12-3.16 (m, 4H, NCH₂×2), 5.05 (s, 2H, NCH₂Ar), 5.22 (s, 2H, NCH₂Ar), 6.79 (d, J=1.6 Hz, 1H, ArH), 6.89 (d, J=1.6 Hz, 1H, ArH), 7.15-7.42 (m, 8H, ArH), 7.60 (m, 1H, ArH); MS(EI) m/e 590 [M⁺+2]; HRMS m/e Cacld. for C₂₆H₂₆N₄O₃S₁Cl₁Br₁ 588.0597, found 588.0590.

### Example 10

### 4-Benzyl-2-(4-bromo-benzyl)-6-chloro-8-(4-methyl-piperazin-1-yl)-1,1-dioxo-1,4-dihydro-2H-1λ⁶-benzo[1,2,4]thiadiazin-3-one

(yield, 66%), white solid; m.p. 115-116° C.; ¹H NMR (200 MHz, CDC₃) δ 2.37 (s, 3H, NCH₃), 2.63-2.68 (m, 4H, NCH₂×2), 3.10-3.15 (m, 4H, NCH₂×2), 5.03 (s, 2H, NCH₂Ar), 5.21 (s, 2H, NCH₂Ar), 6.78 (d, J=1.6 Hz, 1H, ArH), 6.88 (d, J=1.6 Hz, 1H, ArH), 7.13-7.47 (m, 9H, ArH); MS(EI) m/e 590 [M⁺+2]; HRMS m/e Cacld. for C₂₆H₂₆N₄O₃S₁Cl₁Br₁ 588.0597, found 589.0591.

### Example 11

### 4-Benzyl-6-chloro-2-(3-iodo-benzyl)-8-(4-methy 1-piperazin-1-yl)-1,1-dioxo-1,4-dihydro-2H-1λ⁶-benzo[1,2,4]thiadiazin-3-one

(yield, 47%), white solid; m.p. 96-98° C.; ¹H NMR (200 MHz, CDC₃) δ 2.38 (s, 3H, NCH₃), 2.63-2.68 (m, 4H, NCH₂×2), 3.12-3.17 (m, 4H, NCH₂×2), 5.03 (s, 2H, NCH₂Ar), 5.22 (s, 2H, NCH₂Ar), 6.79 (d, J=1.6 Hz, 1H, ArH), 6.89 (d, J=1.6 Hz, 1H, ArH), 7.02-7.19 (m, 3H, ArH), 7.27-7.45 (m, 4H, ArH), 7.62 (m, 1H, ArH), 7.80 (m, 1H, ArH); MS(EI) m/e 636 [M⁺]; HRMS m/e Cacld. for C₂₆H₂₆N₄O₃S₁Cl₁I₁ 636.0459, found 636.0457.

### Example 12

### 4-Benzyl-6-chloro-2-(4-iodo-benzyl)-8-(4-methyl-piperazin-1-yl)-1,1-dioxo-1,4-dihydro-2H-1λ⁶-benzo[1,2,4]thiadiazin-3-one

(yield, 61%), white solid; m.p. 110-111° C.; ¹H NMR (200 MHz, CDC₃) δ 2.37 (s, 3H, NCH₃), 2.63-2.66 (m, 4H, NCH₂×2), 3.12-3.16 (m, 4H, NCH₂×2), 5.02 (s, 2H, NCH₂Ar), 5.21 (s, 2H, NCH₂Ar), 6.77 (d, J=2.0 Hz, 1H, ArH), 6.87 (d, J=2.0 Hz, 1H, ArH), 7.13-7.32 (m, 7H, ArH), 7.63-7.67 (m, 2H, ArH); MS(EI) m/e 636 [M⁺]; HRMS m/e Cacld. for C₂₆H₂₆N₄O₃S₁Cl₁I₁ 636.0458, found 636.0458.

### Example 13

### 4-Benzyl-6-chloro-2-(2-methyl-benzyl)-8-(4-methyl-piperazin-1-yl)-1,1-dioxo-1,4-dihydro-2H-1λ⁶-benzo[1,2,4]thiadiazin-3-one

(yield, 71%), white solid; m.p. 148-150° C.; ¹H NMR (200 MHz, CDC₃) δ 2.35 (s, 3H, NCH₃), 2.38 (s, 3H, CH₃), 2.60-2.64 (m, 4H, NCH₂×2), 3.12-3.16 (m, 4H, NCH₂×2), 5.13 (s, 2H, NCH₂Ar), 5.23 (s, 2H, NCH₂Ar), 6.81 (d, J=1.8 Hz, 1H, ArH), 6.89 (d, J=1.8 Hz, 1H, ArH), 7.14-7.31 (m, 9H, ArH); HRMS m/e Cacld. for C₂₇H₂₉Cl₁N₄O₃S₁ 524.1652, found 524.1649.

### Example 14

### 4-Benzyl-6-chloro-2-(3-methyl-benzyl)-8-(4-methyl-piperazin-1-yl)-1,1-dioxo-1,4-dihydro-2H-1λ⁶-benzo[1,2,4]thiadiazin-3-one

(yield, 52%), m.p. 188-191° C.; ¹H NMR (200 MHz, CDC₃) δ 2.33 (s, 3H, CH₃), 2.38 (s, 3H NCH₃), 2.63-2.68 (m, 4H, NCH₂×2), 3.12-3.18 (m, 4H, NCH₂×2), 5.07 (s, 2H, NCH₂Ar), 5.22 (s, 2H, NCH₂Ar), 6.77 (d, J=1.8 Hz, 1H, ArH), 6.87 (d, J=1.8 Hz, 1H, ArH), 7.07-7.32 (m, 9H, ArH); HRMS m/e Cacld. for C₂₇H₂₉Cl₁N₄O₃S₁ 524.1636, found 524.1649.

### Example 15

### 4-Benzyl-6-chloro-2-(4-methyl-benzyl)-8-(4-methyl-piperazin-1-yl)-1,1-dioxo-1,4-dihydro-2H-1λ⁶-benzo[1,2,4]thiadiazin-3-one

(yield, 65%), white solid; m.p. 180-185° C.; ¹H NMR (200 MHz, CDC₃) δ 2.32 (s, 3H, NCH₃), 2.39 (s, 3H, CH₃), 2.65-2.70 (m, 4H, NCH₂×2), 3.13-3.19 (m, 4H, NCH₂×2), 5.06 (s, 2H, NCH₂Ar), 5.21 (s, 2H, NCH₂Ar), 6.76 (d, J=1.6 Hz, 1H, ArH), 6.87 (d, J=1.6 Hz, 1H, ArH), 7.11-7.18 (m, 3H, ArH), 7.26-7.39 (m, 6H, ArH); HRMS m/e Cacld. for C₂₇H₂₉Cl₁N₄O₃S₁ 524.1643, found 524.1649.

### Example 16

### 4-Benzyl-6-chloro-2-(2-methoxy-benzyl)-8-(4-methyl-piperazin-1-yl)-1,1-dioxo-1,4-dihydro-2H-1λ⁶-benzo[1,2,4]thiadiazin-3-one

(yield, 67%), white solids; m.p. 93-94° C.; ¹H NMR (200 MHz, CDC₃) δ 2.35 (s, 3H, NCH₃), 2.62-2.64 (m, 4H, NCH₂×2), 3.09-3.13 (m, 4H, NCH₂×2), 3.76 (s, 3H, OCH₃), 5.18 (s, 2H, NCH₂Ar), 5.22 (s, 2H, NCH₂Ar), 6.79-6.93 (m, 4H, ArH), 7.16-7.31 (m, 7H, ArH); MS(EI) m/e 540 [M⁺]; HRMS m/e Cacld. for C₂₇H₂₉N₄O₄S₁Cl₁ 1540.1598, found 540.1587.

### Example 17

### 4-Benzyl-6-chloro-2-(3-methoxy-benzyl)-8-(4-methyl-piperazin-1-yl)-1,1-dioxo-1,4-dihydro-2H-1λ⁶-benzo[1,2,4]thiadiazin-3-one

(yield, 64%), white solid; m.p. 85-86° C.; ¹H NMR (200 MHz, CDC₃) δ 2.44 (s, 3H, NCH₃), 2.74-2.76 (m, 4H, NCH₂×2), 3.19-3.22 (m, 4H, NCH₂×2), 3.79 (s, 3H, OCH₃), 5.12 (s, 2H, NCH₂AR), 5.25 (s, 2H, NCH₂Ar), 6.82-6.92 (m, 3H, ArH), 7.04-7.07 (m, 2H, ArH), 7.18-7.34 (m, 6H, ArH); MS(EI) m/e 539 [M⁺−1]; HRMS m/e Cacld. for C₂₇H₂₉N₄O₄S₁Cl₁ 540.1598, found 540.159.

### Example 18

### 4-Benzyl-6-chloro-2-(4-methoxy-benzyl)-8-(4-methyl-piperazin-1-yl)-1,1-dioxo-1,4-dihydro-2H-1λ⁶-benzo[1,2,4]thiadiazin-3-one

(yield, 60%), white solids; m.p.; ¹H NMR (200 MHz, CDC₃) δ 2.40 (s, 3H, NCH₃), 2.70-2.72 (m, 4H, NCH₂×2), 3.14-3.16 (m, 4H, NCH₂×2), 3.78 (s, 3H, OCH₃), 5.05 (s, 2H, NCH₂Ar), 5.21 (s, 2H, NCH₂Ar), 6.76 (d, J=1.6 Hz, 1H, ArH), 6.83-6.87 (m, 3H, ArH), 7.14-7.34 (m, 5H, ArH), 7.42-7.46 (m, 2H, ArH); MS(EI) m/e 540 [M⁺]; HRMS m/e Cacld. for C₂₇H₂₉N₄O₄S₁Cl₁ 540.1598, found 540.1579.

### Example 19

### 4-Benzyl-8-chloro-2-(3-hydroxy-benzyl)-6-(4-methyl-piperazin-1-yl)-1,1-dioxo-1,4-dihydro-2H-1λ⁶-benzo[1,2,4]thiadiazin-3-one

To a solution of Example 17 (0.10 g, 0.19 mmol) in methylene chloride (10□) was added BBr₃ (1M solution in methylene chloride, 0.57□) at −78° C. The resulting mixture was warmed up to room temperature and stirred for 3 h. The reaction mixture was quenched by the addition of water (10□) and then the organic layer was washed with brine (10□), dried over anhydrous MgSO₄ and evaporated in vacuo. The crude residue was purified by a flash column chromatography (CH₂Cl₂:CH₃OH=20:1) to give the title compound (0.070 g, 70%) as a white solid: m.p. 170-172° C.; ¹H NMR MHz, CDC₃) δ 2.47 (s, 3H, NCH₃), 2.81-2.85 (m, 4H, NCH₂×2), 3.14-3.18 (m, 4H, NCH₂×2), 5.00 (s, 2H, NCH₂Ar), 5.17 (s, 2H, NCH₂Ar), 6.75 (d, J=2.0 Hz, 1H, ArH), 6.86 (d, J=2.0 Hz, 1H, ArH), 6.81-6.91 (m, 2H, ArH), 7.10-7.30 (m, 7H, ArH); MS(EI) m/e 526 [M⁺]; HRMS m/e Cacld. for C₂₆H₂₇N₄O₄S₁Cl₁ 526.1441, found 526.1449.

### Example 20

### 4-Benzyl-6-chloro-8-(4-methyl-piperazin-1-yl)-2-(2-nitro-benzyl)-1,1-dioxo-1,4-dihydro-2H-1λ⁶-benzo[1,2,4]thiadiazin-3-one

(yield, 45%), pale yellow solid; m.p. 84-86° C.; ¹H NMR (200 MHz, CDC₃) δ 2.37 (s, 3H, NCH₃), 2.63-2.67 (m, 4H, NCH₂×2), 3.14-3.18 (m, 4H, NCH₂×2), 5.02 (s, 2H, NCH₂Ar), 5.21 (s, 2H, NCH₂Ar), 6.78 (d, J=1.6 Hz, 1H, ArH), 6.87 (d, J=1.6 Hz, 1H, ArH), 7.14-7.33 (m, 8H, ArH), 7.65 (m, 1H, ArH); MS(EI) m/e 555 [M⁺]; HRMS m/e Cacld. for C₂₆H₂₆N₅O₅S₁Cl₁ 555.1343, found 555.1354.

### Example 21

### 4-Benzyl-6-chloro-8-(4-methyl-piperazin-1-yl)-2-(3-nitro-benzyl)-1,1-dioxo-1,4-dihydro-2H-1λ⁶-benzo[1,2,4]thiadiazin-3-one

(yield, 24%), pale yellow solid; m.p. 172-175° C.; ¹H NMR (200 MHz, CDC₃) δ 2.38 (s, 3H, NCH₃), 2.63-2.67 (m, 4H, NCH₂×2), 3.13-3.17 (m, 4H, NCH₂×2), 5.18 (s, 2H, NCH₂Ar), 5.22 (s, 2H, NCH₂Ar), 6.82 (d, J=1.6 Hz, 1H, ArH), 6.91 (d, J=1.6 Hz, 1H, ArH), 7.17-7.38 (m, 5H, ArH), 7.52 (dd, J=7.6, 8.2 Hz, 1H, ArH), 8.14 (m, 1H, ArH), 8.35 (m, 1H, ArH); HRMS m/e Cacld. for C₂₆H₂₆Cl₁N₅O₅S₁ 555.1343, found 555.1367.

### Example 22

### 4-Benzyl-6-chloro-8-(4-methyl-piperazin-1-yl)-2-(4-nitro-benzyl)-1,1-dioxo-1,4-dihydro-2H-1λ⁶-benzo[1,2,4]thiadiazin-3-one

(yield, 57%), pale yellow solid; m.p. 163-164° C.; ¹H NMR (200 MHz, CDC₃) δ 2.37 (s, 3H, NCH₃), 2.62-2.66 (m, 4H, NCH₂×2), 3.13-3.17 (m, 4H, NCH₂×2), 5.16 (s, 2H, NCH₂Ar), 5.22 (s, 2H, NCH₂Ar), 6.82 (d, J=1.2 Hz, 1H, ArH), 6.91 (d, J=1.2 Hz, 1H, ArH), 7.15-7.19 (m, 2H, ArH), 7.26-7.33 (m, 3H, ArH), 7.63-7.67 (m, 2H, ArH), 8.17-8.21 (m, 2H, ArH); MS(EI) m/e 555 [M⁺]; HRMS m/e Cacld. for C₂₆H₂₆N₅O₅S₁Cl₁ 1555.1343, found 555.1336.

### Example 23

### 4-[4-Benzyl-6-chloro-8-(4-methyl-piperazin-1-yl)-1,1,3-trioxo-3,4-dihydro-1H-1λ⁶-benzo[1,2,4]thiadiazin-2-ylmethyl]-benzoic acid methyl ester

(yield, 52%), pale yellow solid; mp 140-143° C.; ¹H NMR (200 MHz, CDC₃) δ 2.38 (s, 3H, NCH₃), 2.62-2.69 (m, 4H, NCH₂×2), 3.12-3.18 (m, 4H, NCH₂×2), 3.90 (s, 3H, OCH₃), 5.14 (s, 2H, NCH₂Ar), 5.22 (s, 2H, NCH₂Ar), 6.80 (d, J=1.6 Hz, 1H, ArH), 6.89 (d, J=1.6 Hz, 1H, ArH), 7.15-7.18 (m, 2H, ArH), 7.28-7.32 (m, 3H, ArH), 7.50-7.54 (m, 2H, ArH), 7.98-8.02 (m, 2H, ArH); HRMS m/e Cacld. for C₂₈H₂₉Cl₁N₄O₅S₁ 568.1547, found 568.1542.

### Example 24

### 4-[4-Benzyl-6-chloro-8-(4-methyl-piperazin-1-yl)-1,1,3-trioxo-3,4-dihydro-2H-1λ⁶-benzo[1,2,4]thiadiazin-2-ylmethyl]-benzonitrile

(yield, 59%), white solid; m.p. 134-135° C.; ¹H NMR (200 MHz, CDC₃) δ 2.37 (s, 3H, NCH₃), 2.62-2.66 (m, 4H, NCH₂×2), 3.12-3.16 (m, 4H, NCH₂×2), 5.11 (s, 2H, NCH₂Ar), 5.21 (s, 2H, NCH₂Ar), 6.81 (d, J=1.6 Hz, 1H, ArH), 6.90 (d, J=1.6 Hz, 1H, ArH), 7.14-7.33 (m, 5H, ArH), 7.56-7.66 (m, 4H, ArH); MS(EI) m/e 535 [M⁺]; HRMS m/e Cacld. for C₂₇H₂₆N₅O₃S₁Cl₁ 535.1444, found 535.1448.

### Example 25

### 4-Benzyl-6-chloro-8-(4-methyl-piperazin-1-yl)-1,1-dioxo-2-[(R)-1-phenyl-ethyl]-1,4-dihydro-2H-1λ⁶-benzo[1,2,4]thiadiazin-3-one

(yield, 55%), white solid; m.p.; ¹H NMR (200 MHz, CDC₃) δ 2.07 (d, J=6.8 Hz, 3H, CH₃), 2.45 (s, 3H, NCH₃), 2.71-2.74 (m, 4H, NCH₂×2), 3.22-3.24 (m, 4H, NCH₂×2), 5.03 (d, J=16.8 Hz, 1H, CHHAr), 5.24 (d, J=16.8 Hz, 1H, CHHAr), 5.86 (q, J=6.8 Hz, 1H, NCHMeAr), 6.77 (d, J=1.6 Hz, 1H, ArH), 6.92 (d, J=1.6 Hz, 1H, ArH), 7.06-7.10 (m, 2H, ArH), 7.30-7.45 (m, 6H, ArH), 7.49-7.53 (m, 2H, ArH); MS(EI) m/e 524 [M⁺+1]; HRMS m/e Cacld. for C₂₇H₃₁Cl₁N₄O₃S₁ 526.1673, found 524.1649.

### Example 26

### 4-Benzyl-6-chloro-8-(4-methyl-piperazin-1-yl)-1,1-dioxo-2-[(S)-1-phenyl-ethyl]-1,4-dihydro-2H-1λ⁶-benzo[1,2,4]thiadiazin-3-one

(yield, 52%), white solid; m.p.; ¹H NMR (200 MHz, CDC₃) δ 2.00 (d, J=7.2 Hz, 3H, CH₃), 2.38 (s, 3H, NCH₃), 2.62-2.69 (m, 4H, NCH₂×2), 3.14-3.19 (m, 4H, NCH₂×2), 4.96 (d, J=16.8 Hz, 1H, CHHAr), 5.17 (d, J=16.8 Hz, 1H, CHHAr), 5.79 (q, J=7.2 Hz, 1H, NCHMeAr), 6.70 (d, J=1.8 Hz, 1H, ArH), 6.86 (d, J=1.8 Hz, 1H, ArH), 6.99-7.03 (m, 2H, ArH), 7.23-7.47 (m, 8H, ArH); MS(EI) m/e 524 [M⁺]; HRMS m/e Cacld. for C₂₇H₂₉Cl₁N₄O₃S₁ 524.1675, found 524.1649.

### Example 27

### 4-Benzyl-6-chloro-8-(4-methyl-piperazin-1-yl)-1,1-dioxo-2-phenyl-1,4-dihydro-2H-1λ⁶-benzo[1,2,4]thiadiazin-3-one

(yield, 55%), solid; m.p.; ¹H NMR (200 MHz, CDC₃) δ 2.33 (s, 3H, NCH₃), 2.62-2.64 (m, 4H, NCH₂×2), 3.16-3.21 (m, 4H, NCH₂×2), 5.31 (s, 2H, NCH₂Ar), 6.94-6.95 (m, 2H, ArH), 7.30-7.38 (m, 6H, ArH), 7.48-7.52 (m, 4H, ArH); HRMS m/e Cacld. for C₂₅H₂₅Cl₁N₄O₃S₁ 496.1324, found 496.1336.

### Example 28

### 4-Benzyl-6-chloro-2-(2-methoxy-phenyl)-8-(4-methyl-piperazin-1-yl)-1,1-dioxo-1,4-dihydro-2H-1λ⁶-benzo[1,2,4]thiadiazin-3-one

(yield, 83%), white solids; m.p. 119-120° C.; ¹H NMR (200 MHz, CDC₃) δ 2.33 (s, 3H, NCH₃), 2.62-2.63 (m, 4H, NCH₂×2), 3.15-3.18 (m, 4H, NCH₂×2), 3.81 (s, 3H, OCH₃), 5.10 (d, J=16.6 Hz, 1H, NCHHAr), 5.40 (d, J=16.6 Hz, 1H, NCHHAr), 6.89-7.13 (m, 4H, ArH), 7.29-7.58 (m, 7H, ArH); MS(EI) m/e 526 [M⁺]; HRMS m/e Cacld. for C₂₆H₂₇N₄O₄S₁Cl₁ 526.1441, found 526.1446.

### Example 29

### 4-Benzyl-6-chloro-2-(3-methoxy-phenyl)-8-(4-methyl-piperazin-1-yl)-1,1-dioxo-1,4-dihydro-2H-1λ⁶-benzo[1,2,4]thiadiazin-3-one

(yield, 41%), white solid; m.p. 191-193° C.; ¹H NMR (200 MHz, CDC₃) δ 2.31 (s, 3H, NCH₃), 2.58-2.62 (m, 4H, NCH₂×2), 3.14-3.18 (m, 4H, CH₂×2), 3.83 (s, 3H, OCH₃), 5.28 (s, 2H, NCH₂Ar), 6.90-6.92 (m, 2H, ArH), 6.97-7.07 (m, 3H, ArH), 7.27-7.44 (m, 6H, ArH); HRMS m/e Cacld. for C₂₆H₂₇Cl₁N₄O₄S₁ 526.1441, found 526.1441.

### Example 30

### 4-Benzyl-6-chloro-2-(4-methoxy-phenyl)-8-(4-methyl-piperazin-1-yl)-1,1-dioxo-1,4-dihydro-2H-1λ⁶-benzo[1,2,4]thiadiazin-3-one

(yield, 66%), white solid; m.p. 115-116° C.; ¹H NMR (200 MHz, CDC₃) δ 2.30 (s, 3H, NCH₃), 2.59-2.61 (m, 4H, NCH₂×2), 3.13-3.18 (m, 4H, NCH₂×2), 3.84 (s, 3H, OCH₃), 5.28 (s, 2H, NCH₂Ar), 6.89-7.02 (m, 5H, ArH), 7.26-7.40 (m, 6H, ArH); MS(EI) m/e 526 [M⁺]; HRMS m/e Cacld. for C₂₆H₂₇N₄O₄S₁Cl₁ 526.1442, found 526.1440.

### Example 31

### 6-Chloro-2,4-dimethyl-8-(4-methyl-piperazin-1-yl)-1,1-dioxo-1,4-dihydro-2H-1λ⁶-benzo[1,2,4]thiadiazin-3-one

(yield, 72%), white solid; m.p.; ¹H NMR (200 MHz, CDC₃) δ 2.41 (s, 3H, NCH₃), 2.66-2.71 (m, 4H, NCH₂×2), 3.19-3.24 (m, 4H, NCH₂×2), 3.35 (s, 3H, NCH₃), 3.49 (s, 3H, NCH₃), 6.92 (d, J=1.6 Hz, 2H, ArH), 6.95 (d, J=1.6 Hz, 2H, ArH); HRMS m/e Cacld. for C₁₄H₁₉Cl₁N₄O₃S 358.0858, found 358.0866.

### Example 32

### 4-Benzyl-6-chloro-2-methyl-8-(4-methyl-piperazin-1-yl)-1,1-dioxo-1,4-dihydro-2H-1λ⁶-benzo[1,2,4]thiadiazin-3-one

(yield, 48%), white solid; m.p.; ¹H NMR (200 MHz, CDC₃) δ 2.39 (s, 3H, NCH₃), 2.57-2.69 (m, 4H, NCH₂×2), 3.14-3.19 (m, 4H, NCH₂×2), 3.39 (s, 3H, NCH₃), 5.24 (s, 2H, NCH₂Ar), 6.81 (d, J=1.6 Hz, 1H, ArH), 6.89 (d, J=1.6 Hz, 1H, ArH), 7.22-7.41 (m, 5H, ArH); HRMS m/e Cacld. for C₂₀H₂₃Cl₁N₄O₃S₁ 434.1174, found 434.1179.

### Example 33

### 4-Benzyl-6-chloro-2-ethyl-8-(4-methyl-piperazin-1-yl)-1,1-dioxo-1,4-dihydro-2H-1λ⁶-benzo[1,2,4]thiadiazin-3-one

(yield, 75%), white solid; m.p. 167-169° C.; ¹H NMR (200 MHz, CDC₃) δ 2.36 (s, 3H, NCH₃), 2.62-2.67 (m, 4H, NCH₂×2), 3.12-3.17 (m, 4H, NCH₂×2), 3.95 (q, J=7.0 Hz, 2H, NCH₂), 5.23 (s, 2H, NCH₂Ar), 6.79 (d, J=1.6 Hz, 1H, ArH), 6.86 (d, J=1.6 Hz, 1H, ArH), 7.21-7.38 (m, 5H, ArH); HRMS m/e Cacld. for C₂₁H₂₅Cl₁N₄O₃S₁ 448.1334, found 448.1336.

### Example 34

### 4-Benzyl-6-chloro-8-(4-methyl-piperazin-1-yl)-2-propyl-1,1-dioxo-1,4-dihydro-2H-1λ⁶-benzo[1,2,4]thiadiazin-3-one

(yield, 59%), white solid; m.p. 91-93° C.; ¹H NMR (200 MHz, CDC₃) δ 0.93 (t, J=7.4 Hz, 3H, CH₃), 1.74-1.93 (m, 2H, CH₂), 2.38 (s, 3H, NCH₃), 2.64-2.69 (m, 4H, NCH₂×2), 3.14-3.18 (m, 2H, CH₂), 3.85 (t, J=7.8 Hz, 2H, NCH₂), 5.23 (s, 2H, NCH₂Ar), 6.80 (d, J=2.0 Hz, 1H, ArH), 6.87 (d, J=2.0 Hz, 1H, ArH), 7.21-7.40 (m, 5H, ArH); HRMS m/e Cacld. for C₂₂H₂₇Cl₁N₄O₃S₁ 462.1491, found 462.1492.

### Example 35

### 4-Benzyl-2-butyl-6-chloro-8-(4-methyl-piperazin-1-yl)-1,1-dioxo-1,4-dihydro-2H-1λ⁶-benzo[1,2,4]thiadiazin-3-one

(yield, 59%), white solid; m.p. 116-118° C.; ¹H NMR (200 MHz, CDC₃) δ 0.92 (t, J=7.4 Hz, 3H, CH₃), 1.40 (m, 2H, CH₂), 1.78 (m, 2H, CH₂), 2.38 (s, 3H, NCH₃), 2.66-2.68 (m, 4H, NCH₂×2), 3.12-3.18 (m, 4H, NCH₂×2), 3.89 (t, J=7.2 Hz, 2H, NCH₂), 5.23 (s, 2H, CH₂Ar), 6.79 (d, J=1.6 Hz, 1H, ArH), 6.87 (d, J=1.6 Hz, 1H, ArH), 7.22-7.39 (m, 5H, ArH); HRMS m/e Cacld. for C₂₃H₂₉ClN₄O₃S₁ 476.1655, found 476.1649.

### Example 36

### 4-Benzyl-6-chloro-2-cyclohexylmethyl-8-(4-methyl-piperazin-1-yl)-1,1-dioxo-1,4-dihydro-2H-1λ⁶-benzo[1,2,4]thiadiazin-3-one

(yield, 77%), white solid; m.p. 155-158° C.; ¹H NMR (200 MHz, CDC₃) δ 0.96-1.26 (m, 6H, CH₂×3 of cyclohexyl), 1.69-1.76 (m, 5H, CH₂×2 and CH of cyclohexyl), 2.37 (s, 3H NCH₃), 2.63-2.67 (m, 4H, NCH₂×2), 3.13-3.20 (m, 4H, NCH₂×2), 3.77 (d, J=7.2 Hz, 2H, NCH₂Cy), 5.22 (s, 2H, NCH₂Ar), 6.79 (d, J=1.6 Hz, 1H, ArH), 6.86 (d, J=1.8 Hz, 1H, ArH), 7.20-7.39 (m, 5H, ArH); HRMS m/e Cacld. for C₂₆H₃₃Cl₁N₄O₃S₁ 519.1956, found 516.1962.

### Example 37

### 4-Benzyl-6-chloro-8-(4-methyl-piperazin-1-yl)-1,1-dioxo-2-phenethyl-1,4-dihydro-2H-1λ⁶-benzo[1,2,4]thiadiazin-3-one

(yield, 48%), white solid; m.p. 151-152° C. ¹H NMR (200 MHz, CDC₃) δ 2.41 (s, 3H, NCH₃), 2.66-2.70 (m, 4H, NCH₂×2), 3.07-3.15 (m, 6H, NCH₂×2, CH₂Ar), 4.19 (t, J=7.8 Hz, 2H, NCH₂), 5.23 (s, 2H, NCH₂Ar), 6.79 (d, J=1.6 Hz, 1H, ArH), 6.89 (d, J=1.6 Hz, 1H, ArH), 7.18-7.38 (m, 10H, ArH); m.p. 151-152° C.; HRMS m/e Cacld. for C₂₇H₂₉Cl₁N₄O₃S₁ 524.1650, found 524.1649.

### Example 38

### 4-Benzyl-6-chloro-2-[3-(3,5-dimethyl-phenyl)-propyl]-8-(4-methyl-piperazin-1-yl)-1,1-dioxo-1,4-dihydro-2H-1λ⁶-benzo[1,2,4]thiadiazin-3-one

(yield, 44%), solid; m.p.; ¹H NMR (200 MHz, CDCl₃) δ 2.13 (m, 2H, CH₂), 2.29 (s, 6H, CH₂×2), 2.40 (s, 3H, NCH₃), 2.60-2.72 (m, 6H, NCH₂×2 & CH₂Ar), 3.17-3.21 (m, 4H, NCH₂×2), 4.01 (m, 2H, NCH₃), 5.25 (s, 2H, NCH₂Ar), 6.80 (d, J=1.8 Hz, 1H, ArH), 6.82 (d, J=2.8 Hz, 2H, ArH), 6.89 (d, J=1.8 Hz, 1H, ArH), 7.22-7.37 (m, 6H, ArH); HRMS m/e Cacld. for C₃₀H₃₅Cl₁N₄O₃S₁ 556.2133, found 556.2118.

### Example 39

### 4-Benzyl-6-chloro-8-(4-methyl-piperazin-1-yl)-2-octyl-1,1-dioxo-1,4-dihydro-2H-1λ⁶-benzo[1,2,4]thiadiazin-3-one

(yield, 58%), white solid; m.p. 88-91° C.; ¹H NMR (200 MHz, CDC₃) δ 0.84 (t, J=6.6 Hz, 3H, CH₃), d 1.26-1.33 (m, 10H, CH₂×5), 1.79 (m, 2H, CH₂), 2.37 (s, 3H, NCH₃), 2.63-2.67 (m, 4H, NCH₂×2), 3.15 (m, 2H, CH₂), 3.87 (t, J=7.2 Hz, 2H, NCH₂), 5.22 (s, 2H, NCH₂Ar), 6.79 (d, J=1.8 Hz, 1H, ArH), 6.86 (d, J=1.8 Hz, 1H, ArH), 7.21-7.38 (m, 5H, ArH); HRMS m/e Cacld. for C₂₇H₂₉Cl₁N₄O₃S₁ 532.2269, found 532.2275.

### Example 40

### 4-Benzyl-6-chloro-2-decyl-8-(4-methyl-piperazin-1-yl)-1,1-dioxo-1,4-dihydro-2H-1λ⁶-benzo[1,2,4]thiadiazin-3-one

(yield, 63%), pale yellow oil; ¹H NMR (200 MHz, CDCl₃) δ 0.83 (t, J=7.0 Hz, 3H, CH of decyl), 1.24-1.32 (m, 14H, CH₂×7 of decyl), 1.79 (m, 2H, CH₂ of decyl), 2.36 (s, 3H, NCH₃), 2.64-2.67 (m, 4H, NCH₂×2), 3.12-3.15 (m, 4H, NCH₂×2), 3.87 (t, J=7.6 Hz, 2H, NCH₂ of decyl), 5.22 (s, 2H, NCH₂Ar), 6.78 (d, J=1.6 Hz, 1H, ArH), 6.85 (d, J=1.6 Hz, 1H, ArH), 7.20-7.34 (m, 5H, ArH); MS(EI) m/e 560 [M⁺]; HRMS m/e Cacld. for C₂₉H₄₁N₄O₃S₁Cl1 560.2587, found 560.2581.

### Example 41

### 4-Benzyl-6-chloro-8-(4-methyl-piperazin-1-yl)-2-naphthalen-1-ylmethyl-1,1-dioxo-1,4-dihydro-2H-1λ⁶-benzo[1,2,4]thiadiazin-3-one

(yield, 78%), white solid; m.p. 114-115° C.; ¹H NMR (200 MHz, CDCl₃) δ 2.38 (s, 3H, NCH₃), 2.66-2.69 (m, 4H, NCH₂×2), 3.15-3.19 (m, 4H, NCH₂×2), 5.25 (s, 2H, NCH₂Ar), 5.66 (s, 2H, NCH₂Ar), 6.79 (d, J=1.6 Hz, 1H, ArH), 6.89 (d, J=1.6 Hz, 1H, ArH), 7.15-7.53 (m, 9H, ArH), 7.79-7.91 (m, 2H, ArH), 8.15 (m, 1H, ArH); MS(EI) m/e 560 [M⁺]; HRMS m/e Cacld. for C₃₀H₂₉N₄O₃S₁Cl₁ 560.1650, found 560.1647.

### Example 42

### 4-Benzyl-6-chloro-8-(4-methyl-piperazin-1-yl)-1,1-dioxo-2-pyridin-4-ylmethyl-1,4-dihydro-2H-1λ⁶-benzo[1,2,4]thiadiazin-3-one

(yield, 41%), pale yellow solid; m.p. 107-108° C.; ¹H NMR (200 MHz, CDCl₃) δ 2.36 (s, 3H, NCH₃), 2.62-2.66 (m, 4H, NCH₂×2), 3.12-3.16 (m, 4H, NCH₂×2), 5.07 (s, 2H, NCH₂Ar), 5.22 (s, 2H, NCH₂Ar), 6.82 (d, J=1.6 Hz, 1H, ArH), 6.91 (d, J=1.6 Hz, 1H, ArH), 7.15-7.36 (m, 7H, ArH), 8.56-8.59 (m, 2H, ArH); MS(EI) m/e 510 [M⁺−1]; HRMS m/e Cacld. for C₂₅H₂₆N₅O₃S₁Cl₁ 511.1444, found 511.1444.

### Example 43

### 4-Benzyl-6-chloro-2-(5-methyl-furan-2-ylmethyl)-8-(4-methyl-piperazin-1-yl)-1,1-dioxo-1,4-dihydro-2H-1λ⁶-benzo[1,2,4]thiadiazin-3-one

(yield, 55%), white solid; m.p. 130-131° C.; ¹H NMR (200 MHz, CDC₃) δ 2.25 (s, 3H, CH₃ of furan), 2.37 (s, 3H, NCH₃), 2.64-2.67 (m, 4H, NCH₂×2), 3.11-3.14 (m, 4H, NCH₂×2), 5.04 (s, 2H, NCH₂Ar), 5.22 (s, 2H, NCH₂Ar), 5.88 (d, J=2.8 Hz, 1H, CH of furanyl), 6.28 (d, J=2.8 Hz, 1H, CH of furanyl), 6.76 (d, J=1.6 Hz, 1H, ArH), 6.85 (d, J=1.6 Hz, 1H, ArH), 7.19-7.33 (m, 5H, ArH); MS(EI) m/e 514 [M⁺]; HRMS m/e Cacld. for C₂₅H₂₇N₄O₅S₁Cl₁ 514.1441, found 514.1426.

### Example 44

### 2,4-Dibenzyl-6-chloro-1,1-dioxo-8-piperazin-1-yl-1,4-dihydro-2H-1λ⁶-benzo[1,2,4]thiadiazin-3-one

(yield, 55%), white solid; m.p. 108-109° C.; ¹H NMR (200 MHz, CDCl₃) δ 3.12-3.15 (m, 8H, NCH₂×4), 5.14 (s, 2H, NCH₂Ar), 5.24 (s, 2H, NCH₂Ar), 6.81 (d, J=1.6 Hz, 1H, ArH), 6.90 (d, J=1.6 Hz, 1H, ArH), 7.17-7.21 (m, 3H, ArH), 7.27-7.39 (m, 5H, ArH), 7.48-7.52 (m, 2H, ArH); MS(EI) m/e 495 [M⁺−1]; HRMS m/e Cacld. for C₂₅H₂₅N₄O₃S₁Cl₁ 496.1336, found 496.1315.

### Example 45

### 4-Benzyl-6-chloro-2-(2-fluoro-benzyl)-1,1-dioxo-8-piperazin-1-yl-1,4-dihydro-2H-1λ⁶-benzo[1,2,4]thiadiazin-3-one

(yield, 66%), pale yellow solid; m.p. 120-121° C.; ¹H NMR (200 MHz, CDCl₃) δ 3.04-3.06 (m, 8H, NCH₂×4), 5.20 (s, 2H, NCH₂Ar), 5.22 (s, 2H, NCH₂Ar), 6.80 (d, J=1.2 Hz, 1H, ArH), 6.87 (d, J=1.2 Hz, 1H, ArH), 6.97-7.44 (m, 9H, ArH); MS(EI) m/e 514 [M⁺]; HRMS m/e Cacld. for C₂₅H₂₄N₄O₃F₁S₁Cl₁ 514.1241, found 514.1235.

### Example 46

### 4-Benzyl-6-chloro-2-(3-fluoro-benzyl)-1,1-dioxo-8-piperazin-1-yl-1,4-dihydro-2H-1λ⁶-benzo[1,2,4]thiadiazin-3-one

(yield, 64%), white solid; mp 154-155° C.; ¹H NMR (200 MHz, CDC₃) δ 3.08-3.10 (m, 8H, NCH₂×4), 5.08 (s, 2H, NCH₂Ar), 5.22 (s, 2H, NCH₂Ar), 6.79 (d, J=1.6 Hz, 1H, ArH), 6.88 (d, J=1.6 Hz, 1H, ArH), 6.92 (m, 1H, ArH), 7.15-7.37 (m, 8H, ArH); HRMS m/e Cacld. for C₂₅H₂₄Cl₁F₁N₄O₃S 514.1242, found 514.1233.

### Example 47

### 4-Benzyl-6-chloro-2-(2-chloro-benzyl)-1,1-dioxo-8-piperazin-1-yl-1,4-dihydro-2H-1λ⁶-benzo[1,2,4]thiadiazin-3-one

(yield, 60%), pale yellow solid; m.p. 126-127° C.; ¹H NMR (200 MHz, CDCl₃) δ 3.05-3.07 (m, 8H, NCH₂×4), 5.24-5.25 (m, 4H, NCH₂Ar×2), 6.84 (d, J=1.6 Hz, 1H, ArH), 6.89 (d, J=1.6 Hz, 1H, ArH), 7.19-7.35 (m, 9H, ArH); MS(EI) m/e 530 [M⁺]; HRMS m/e Cacld. for C₂₅H₂₄N₄O₃S₁Cl₂ 1530.0946, found 530.0947.

### Example 48

### 4-Benzyl-2-(2-bromo-benzyl)-6-chloro-1,1-dioxo-8-piperazin-1-yl-1,4-dihydro-2H-1λ⁶-benzo[1,2,4]thiadiazin-3-one

(yield, 51%), white solid; m.p. 144-145° C.; ¹H NMR (200 MHz, CDC₃) δ 3.03-3.05 (m, 8H, NCH₂×4), 5.21 (s, 2H, NCH₂Ar), 5.24 (s, 2H, NCH₂Ar), 6.84 (d, J=1.6 Hz, 1H, ArH), 6.89 (d, J=1.6 Hz, 1H, ArH), 7.09-7.32 (m, 8H, ArH), 7.53 (m, 1H, ArH); MS(EI) m/e 576 [M⁺+2]; HRMS m/e Cacld. for C₂₅H₂₄N₄O₃S₁Cl₁Br₁ 574.0441, found 574.0440.

### Example 49

### 4-Benzyl-6-chloro-2-(4-iodo-benzyl)-1,1-dioxo-8-piperazin-1-yl-1,4-dihydro-2H-1λ⁶-benzo[1,2,4]thiadiazin-3-one

(yield, 58%), white solid; m.p. 153-154° C.; ¹H NMR (200 MHz, CDC₃) δ 3.06-3.08 (m, 8H, NCH₂×4), 5.02 (s, 2H, NCH₂Ar), 5.20 (s, 2H, NCH₂Ar), 6.77 (d, J=1.6 Hz, 1H, ArH), 6.87 (d, J=1.6 Hz, 1H, ArH), 7.14-7.32 (m, 7H, ArH), 7.63-7.67 (m, 2H, ArH); MS(EI) m/e 622 [M⁺]; HRMS m/e Cacld. for C₂₅H₂₄N₄O₃S₁Cl₁I₁ 11622.0302, found 622.0306.

### Example 50

### 4-Benzyl-6-chloro-2-(2-methyl-benzyl)-1,1-dioxo-8-piperazin-1-yl-1,4-dihydro-2H-1λ⁶-benzo[1,2,4]thiadiazin-3-one

(yield, 58%), white solid; m.p. 70-72° C.; ¹H NMR (200 MHz, CDCl₃) δ 2.38 (s, 3H, CH₃), 3.07-3.09 (m, 8H, NCH₂×4), 5.23 (s, 2H, NCH₂Ar), 5.30 (s, 2H, NCH Ar), 6.82 (d, J=1.8 Hz, 1H, ArH), 6.88 (d, J=1.8 Hz, 1H, ArH), 7.14-7.31 (m, 9H, ArH); HRMS m/e Cacld. for C₂₆H₂₇Cl₁N₄O₃S₁ 510.1486, found 510.1492.

### Example 51

### 4-Benzyl-6-chloro-2-(2-methoxy-benzyl)-1,1-dioxo-8-piperazin-1-yl-1,4-dihydro-2H-1λ⁶-benzo[1,2,4]thiadiazin-3-one

(yield, 65%); white solids; m.p. 129-130° C.; ¹H NMR (200 MHz, CDC₃) δ 3.05-3.12 (m, 8H, NCH₂×4), 3.76 (s, 3H, OCH₃), 5.18 (s, 2H, NCH₂Ar), 5.22 (s, 2H, NCH₂ Ar), 6.79-6.89 (m, 4H, ArH), 7.16-7.32 (m, 7H, ArH); MS(EI) m/e 526 [M⁺].

### Example 52

### 4-Benzyl-6-chloro-2-(3-methoxy-benzyl)-1,1-dioxo-8-piperazin-1-yl-1,4-dihydro-2H-1λ⁶-benzo[1,2,4]thiadiazin-3-one

(yield, 61% %), white solid; m.p. 98-100° C.; ¹H NMR (200 MHz, CDC₃) δ 3.12-3.16 (m, 8H, NCH₂×4), 3.78 (s, 3H, OCH₃), 5.11 (s, 2H, NCH₂Ar), 5.24 (s, 2H, NCH₂ Ar), 6.81-6.91 (m, 3H, ArH), 7.03-7.07 (m, 2H, ArH), 7.18-7.34 (m, 6H, ArH); MS(EI) m/e 526 [M⁺].

### Example 53

### 4-Benzyl-6-chloro-2-(4-methoxy-benzyl)-1,1-dioxo-8-piperazin-1-yl-1,4-dihydro-2H-1λ⁶-benzo[1,2,4]thiadiazin-3-one

(yield, 67%), white solids; m.p.; ¹H NMR (200 MHz, CDC₃) δ 3.11-3.15 (m, 8H, NCH₂×4), 3.81 (s, 3H, OCH₃), 5.07 (s, 2H, NCH₂Ar), 5.24 (s, 2H, NCH₂Ar), 6.79 (d, J=1.6 Hz, 1H, ArH), 6.85-6.90 (m, 3H, ArH), 7.17-7.21 (m, 2H, ArH), 7.29-7.34 (m, 3H, ArH), 7.44-7.49 (m, 2H, ArH); MS(EI) m/e 526 [M⁺]; HRMS m/e Cacld. for C₂₆H₂₇N₄O₄S₁Cl₁ 526.1442, found 526.1446.

### Example 54

### 4-Benzyl-6-chloro-2-(3-hydroxy-benzyl)-1,1-dioxo-8-piperazin-1-yl-1,4-dihydro-2H-1λ⁶-benzo[1,2,4]thiadiazin-3-one

To a solution of Example 52 (0.05 g, 0.10 mmol) in methylene chloride (5□) was added boron tribromide (0.28 □, 1.0 M solution in methylene chloride) at −78° C. The resulting solution was warmed up to room temperature and stirred for 4 hours. The reaction mixture was poured into cold water (100□) and extracted with ethyl acetate (100 □×2). The organic layer was washed with brine, dried over anhydrous MgSO₄ and concentrated in vacuo. The residue was purified by a flash column chromatography (eluent, 10:1 mixture of CH₂Cl₂ and CH₃OH) to afford the pure title compound (0.031 g, 63%) as a white solid: m.p. 230-231° C.; ¹H NMR (200 MHz, CDC₃) δ 3.13-3.18 (m, 8H, NCH₂×4), 5.00 (s, 2H, NCH₂Ar), 5.20 (s, 2H, NCH₂Ar), 5.54 (br s, 1H, NH), 6.73-7.06 (m, 7H, ArH), 7.15-7.35 (m, 4H, ArH); MS(EI) m/e 512 [M⁺]; HRMS m/e Cacld. for.

### Example 55

### 4-Benzyl-6-chloro-2-(2-nitro-benzyl)-1,1-dioxo-8-piperazin-1-yl-1,4-dihydro-2H-1λ⁶-benzo[1,2,4]thiadiazin-3-one

(yield, 57%), pale yellow solid; mp 134-135° C.; ¹H NMR (200 MHz, CDC₃) δ 3.04-3.08 (m, 8H, NCH₂×4), 5.24 (s, 2H, NCH₂Ar), 5.54 (s, 2H, NCH₂Ar), 6.86 (d, J=1.2 Hz, 1H, ArH), 6.91 (d, J=1.2 Hz, 1H, ArH), 7.20-7.49 (m, 6H, ArH), 7.57-7.63 (m, 2H, ArH), 8.08 (m, 1H, ArH); HRMS m/e Cacld. for C₂₅H₂₄Cl₁N₅O₅S₁ 541.1187, found 541.1181.

### Example 56

### 4-(4-Benzyl-6-chloro-1,1,3-trioxo-8-piperazin-1-yl-3,4-dihydro-1H-1λ⁶-benzo[1,2,4]thiadiazin-2-ylmethyl)-benzoic acid methyl ester

(yield, 40%), pale yellow solid; m.p. 136-139° C.; ¹H NMR (200 MHz, CDCl₃) δ 3.12-3.15 (m, 8H, NCH₂×4), 3.89 (s, 3H, OCH₃), 5.14 (s, 2H, NCH₂Ar), 5.22 (s, 2H, NCH₂Ph), 6.81 (d, J=2.0 Hz, 1H, ArH), 6.89 (d, J=2.0 Hz, 1H, ArH), 7.14-7.36 (m, 5H, ArH), 7.49-7.57 (m, 2H, ArH), 7.98-8.05 (m, 2H, ArH); HRMS m/e Cacld. for C₂₇H₂₇Cl₁N₄O₅S₁ 554.1391, found 554.1388.

### Example 57

### 4-(4-Benzyl-6-chloro-1,1,3-trioxo-8-piperazin-1-yl-3,4-dihydro-1H-1λ⁶-benzo[1,2,4]thiadiazin-2-ylmethyl)-benzonitrile

(yield, 58%), shiny white solid; mp 135-137° C.; ¹H NMR (200 MHz, CDC₃) δ 3.06-3.09 (m, 8H, NCH₂×4), 5.12 (s, 2H, NCH₂Ar), 5.22 (s, 2H, NCH₂Ar), 6.81 (d, J=1.6 Hz, 1H, ArH), 6.90 (d, J=1.6 Hz, 1H, ArH), 7.15-7.19 (m, 2H, ArH), 7.26-7.33 (m, 3H, ArH), 7.56-7.67 (m, 4H, ArH); HRMS m/e Cacld. for C₂₆H₂₄Cl₁N₅O₃S₁ 521.1288, found 521.1282.

### Example 58

### 4-Benzyl-6-chloro-1,1-dioxo-2-[(R)-1-phenyl-ethyl]-8-piperazin-1-yl-1,4-dihydro-2H-1λ⁶-benzo[1,2,4]thiadiazin-3-one

(yield, 55%), white solid; m.p.; ¹H NMR (200 MHz, CDC₃) δ 2.04 (d, J=6.8 Hz, 3H, CH₃), 3.08-3.18 (m, 8H, NCH₂×4), 5.02 (d, J=16.3 Hz, 1H, NCHHAr), 5.24 (d, J=16.3 Hz, 1H, NCHHAr), 5.83 (q, J=6.8 Hz, 1H, NCHMeAr), 6.73 (d, J=2.0 Hz, 1H, ArH), 6.88 (d, J=2.0 Hz, 1H, ArH), 7.01-7.06 (m, 2H, ArH), 7.25-7.35 (m, 6H, ArH), 7.44-7.49 (m, 2H, ArH); MS(EI) m/e 510 [M⁺]; HRMS m/e Cacld. for C₂₆H₂₇Cl₁N₄O₃S₁ 510.1494, found 510.1492.

### Example 59

### 4-Benzyl-6-chloro-1,1-dioxo-2-[(S)-1-phenyl-ethyl]-8-piperazin-1-yl-1,4-dihydro-2H-1λ⁶-benzo[1,2,4]thiadiazin-3-one

(yield, 51%), white solid; m.p.; ¹H NMR (200 MHz, CDC₃) δ 2.03 (d, J=6.8 Hz, 3H, CH₃), 3.14 (m, 8H, NCH₂×4), 5.02 (d, J=16.4 Hz, 1H, NCHHAr), 5.22 (d, J=16.4 Hz, 1H, NCHHAr), 5.86 (q, J=6.8 Hz, 1H, NCHMeAr), 6.73 (d, J=1.6 Hz, 1H, ArH), 6.87 (d, J=1.6 Hz, 1H, ArH), 7.00-7.04 (m, 2H, ArH), 7.29-7.37 (m, 6H, ArH), 7.43-7.46 (m, 2H, ArH); MS(EI) m/e 510 [M⁺]; HRMS m/e Cacld. for C₂₆H₂₇Cl₁N₄O₃S₁ 510.1492, found 510.1488.

### Example 60

### 4-Benzyl-6-chloro-1,1-dioxo-2-phenyl-8-piperazin-1-yl-1,4-dihydro-2H-1λ⁶-benzo[1,2,4]thiadiazin-3-one

(yield, 61%), white solid; m.p. 275-277° C.; ¹H NMR (200 MHz, CDCl₃) δ 3.11 (m, 8H, NCH₂×4), 5.30 (s, 2H, CH₂Ar), 6.92-6.96 (m, 2H, ArH), 7.33-7.38 (m, 5H, ArH), 7.48-7.54 (m, 5H, ArH); MS(EI) m/e 482 [M⁺]; HRMS m/e Cacld. for C₂₄H₂₃Cl₁N₄O₃S₁ 482.1175, found 482.1179.

### Example 61

### 4-Benzyl-6-chloro-2-(2-methoxy-phenyl)-1,1-dioxo-8-piperazin-1-yl-1,4-dihydro-2H-1λ⁶-benzo[1,2,4]thiadiazin-3-one

(yield, 61%), white solid; m.p. 175-176° C.; ¹H NMR (200 MHz, CDC₃) δ 2.65-2.71 (m, 4H, NCH₂×2), 3.02-3.09 (m, 4H, NCH₂×2), 3.78 (s, 3H, OCH₃), 5.07 (d, J=16.8 Hz, 1H, NCHHAr), 5.37 (d, J=16.8 Hz, 1H, NCHHAr), 6.86-7.11 (m, 4H, ArH), 7.27-7.57 (m, 7H, ArH); MS(EI) m/e 512 [M⁺]; HRMS m/e Cacld. for C₂₅H₂₅N₄O₄S₁Cl₁ 1512.1285, found 512.1276.

### Example 62

### 4-Benzyl-6-chloro-2-(3-methoxy-phenyl)-1,1-dioxo-8-piperazin-1-yl-1,4-dihydro-2H-1λ⁶-benzo[1,2,4]thiadiazin-3-one

(yield, 59%), solids; m.p.; ¹H NMR (200 MHz, CDC₃) δ 3.02-3.18 (m, 8H, NCH 2×4), 3.85 (s, 3H, OCH₃), 5.30 (s, 2H, NCH₂Ar), 6.94 (m, 2H, ArH), 7.01-7.09 (m, 3H, ArH), 7.30-7.46 (m, 6H, ArH); MS(EI) m/e 512 [M⁺]; HRMS m/e Cacld. for.

### Example 63

### 4-Benzyl-6-chloro-2-(4-methoxy-phenyl)-1,1-dioxo-8-piperazin-1-yl-1,4-dihydro-2H-1λ⁶-benzo[1,2,4]thiadiazin-3-one

(yield, 65%), white solids; m.p. 155-156° C.; ¹H NMR (200 MHz, CDC₃) δ 3.06-3.09 (m, 8H, NCH₂×4), 3.84 (s, 3H, OCH₃), 5.28 (s, 2H, NCH₂Ar), 6.98-7.02 (m, 4H, ArH), 7.26-7.40 (m, 7H, ArH); MS(EI) m/e 512 [M⁺]; HRMS m/e Cacld. for C₂₅H₂₅N₄O₄S₁Cl₁ 1512.1285, found 512.1283.

### Example 64

### 4-Benzyl-6-chloro-2-ethyl-1,1-dioxo-8-piperazin-1-yl-1,4-dihydro-2H-1λ⁶-benzo[1,2,4]thiadiazin-3-one

(yield, 43%), white solid; m.p. 158-161° C.; ¹H NMR (200 MHz, CDC₃) δ 1.38 (t, J=7.0 Hz, 3H, CH₃), 3.09 (m, 8H, NCH₂×4), 3.96 (q, J=7.0 Hz, 2H, NCH₂), 5.24 (s, 2H, NCH₂Ph), 6.79 (d, J=1.6 Hz, 1H, ArH), 6.86 (d, J=1.6 Hz, 1H, ArH), 7.22-7.40 (m, 5H, ArH); HRMS m/e Cacld. for C₂₀H₂₃Cl₁N₄O₃S₁ 434.1179, found 434.1179.

### Example 65

### 4-Benzyl-6-chloro-2-propyl-1,1-dioxo-8-piperazin-1-yl-1,4-dihydro-2H-1λ⁶-benzo[1,2,4]thiadiazin-3-one

(yield, 54%), white solid; m.p. 148-150° C.; ¹H NMR (200 MHz, CDC₃) δ 0.93 (t, J=7.2 Hz, 3H, CH₃), 1.78-1.89 (m, 2H, CH₂), 3.09 (m, 8H, NCH₂×4), 3.86 (t, J=7.4 Hz, 2H, NCH₂), 5.32 (s, 2H, NCH₂Ph), 6.81 (d, J=2.0 Hz, 1H, ArH), 6.87 (d, J=2.0 Hz, 1H, ArH), 7.22-7.35 (m, 5H, ArH); HRMS m/e Cacld. for C₂₁H₂₅Cl₁N₄O₃S₁ 448.1336, found 448.1338.

### Example 66

### 4-Benzyl-6-chloro-2-[3-(3,5-dimethyl-phenyl)-propyl]-1,1-dioxo-8-piperazin-1-yl-1,4-dihydro-2H-1λ⁶-benzo[1,2,4]thiadiazin-3-one

(yield, 75%), white solid; m.p. 226-228° C.; ¹H NMR (200 MHz, CDC₃) δ 2.07-2.18 (m, 2H, CH₂), 2.29 (s, 6H, CH₂×2), 2.65 (t, J=7.9 Hz, 2H, CH₂Ar), 3.42 (m, 8H, NCH₂×4), 4.01 (m, 2H, NCH₃), 5.26 (s, 2H, NCH₂Ar), 6.84 (m, 3H, ArH), 6.89 (d, J=2.2 Hz, 1H, ArH), 6.92 (d, J=2.2 Hz, 1H, ArH), 7.22-7.40 (m, 5H, ArH); HRMS m/e Cacld. for C₂₉H₃₃Cl₁N₄O₃S₁ 552.1962, found 552.1979.

### Example 67

### 4-Benzyl-6-chloro-2-decyl-1,1-dioxo-8-piperazin-1-yl-1,4-dihydro-2H-1λ⁶-benzo[1,2,4]thiadiazin-3-one

(yield, 64%), colorless oil; ¹H NMR (200 MHz, CDCl₃) δ 0.84 (t, J=7.0 Hz, 3H, CH₃ of decyl), 1.25-1.33 (m, 12H, CH₂×6 of decyl), 1.77-1.79 (m, 4H, CH₂×2 of decyl), 3.07-3.09 (m, 8H, NCH₂×4), 3.87 (t, J=7.6 Hz, 2H, NCH₂ of decyl), 5.22 (s, 2H, NCH₂Ar), 6.78 (d, J=1.6 Hz, 1H, ArH), 6.85 (d, J=1.6 Hz, 1H, ArH), 7.21-7.39 (m, 5H, ArH); HRMS m/e Cacld. for C₂₈H₃₉Cl₁N₄O₃S 546.2431, found 546.2428.

### Example 68

### 4-Benzyl-6-chloro-2-naphthalen-1-ylmethyl-1,1-dioxo-8-piperazin-1-yl-1,4-dihydro-2H-1λ⁶-benzo[1,2,4]thiadiazin-3-one

(yield, 68%), white solid; m.p. 164-166° C.; ¹H NMR (200 MHz, CDC₃) δ 3.00-3.16 (m, 8H, NCH₂×4), 5.22 (s, 2H, NCH₂Ar), 5.64 (s, 2H, NCH₂Ar), 6.77 (d, J=1.8 Hz, 1H, ArH), 6.85 (d, J=1.8 Hz, 1H, ArH), 7.12-7.16 (m, 2H, ArH), 7.26-7.34 (m, 3H, ArH), 7.36-7.50 (m, 4H, ArH), 7.75-7.87 (m, 2H, ArH), 8.12 (m, 1H, ArH); HRMS m/e Cacld. for C₂₉H₂₇Cl₁N₄O₃S 546.1492, found 546.1496.

### Example 69

### 4-Benzyl-6-chloro-2-(2-chloro-benzyl)-8-morpholin-4-yl-1,1-dioxo-1,4-dihydro-2H-1λ⁶-benzo[1,2,4]thiadiazin-3-one

(yield, 52%), white solid; m.p. 171-172° C.; ¹H NMR (200 MHz, CDC₃) δ 3.09 (m, 4H, CH₂N×2), 3.87 (m, 4H, CH₂O×2), 5.25 (s, 4H, NCH₂Ar×2), 6.87 (d, J=1.5 Hz, 1H, ArH), 6.90 (d, J=1.5 Hz, 1H, ArH), 7.19-7.38 (m, 9H, ArH); HRMS m/e Cacld. for C₂₅H₂₃Cl₂N₃O₄S₁ 531.0786, found 531.0802.

### Example 70

### 4-Benzyl-6-chloro-2-(2-chloro-benzyl)-1,1-dioxo-8-(4-pyridin-2-yl-piperazin-1-yl)-1,4-dihydro-2H-1λ⁶-benzo[1,2,4]thiadiazin-3-one

(yield, 55%), white solid; m.p. 159-161° C.; ¹H NMR (200 MHz, CDC₃) δ 3.23 (m, 4H, CH₂NPy×2), 3.76 (br, 4H, CH₂NAr×2), 5.28 (s, 4H, NCH₂Ar×2), 6.64-6.71 (m, 2H, ArH), 6.89-6.96 (m, 2H, ArH), 7.22-7.41 (m, 9H, ArH), 7.47-7.56 (m, 1H, ArH), 8.20-8.24 (m, 1H, ArH); HRMS m/e Cacld. for C₃₀H₂₇Cl₂N₅O₃S₁ 607.1212, found 607.1235.

### Example 71

### 4-Benzyl-6-chloro-2-(2-chloro-benzyl)-8-[(R)-3-methyl-piperazin-1-yl ]-1,1-dioxo-1,4-dihydro-2H-1λ⁶-benzo[1,2,4]thiadiazin-3-one

(yield, 37%), white solid; m.p. 134-136° C.; ¹H NMR (200 MHz, CDC₃) δ 1.06 (d, J=6.6 Hz, 3H, CH₃), 2.45 (m, 1H, CHNH), 2.80 (m, 1H, CHHNH), 3.02 (m, 1H, CHHNH), 3.20 (m, 4H, CH₂N×2), 5.24 (s, 4H, NCH₂Ar×2), 6.84 (d, J=1.7 Hz, 1H, ArH), 6.89 (d, J=1.7 Hz, 1H, ArH), 7.20-7.37 (m, 9H, ArH); HRMS m/e Cacld. for C₂₆H₂₆Cl₂N₄O₃S₁ 544.1103, found 544.1124.

### Example 72

### 4-Benzyl-6-chloro-2-(2-chloro-benzyl)-8-[(S)-3-methyl-piperazin-1-yl ]-1,1-dioxo-1,4-dihydro-2H-1λ⁶-benzo[1,2,4]thiadiazin-3-one

(yield, 73%), white solid; m.p. 142-143° C.; ¹H NMR (200 MHz, CDC₃) δ 1.06 (d, J=6.1 Hz, 3H, CH₃), 2.44 (t, J=10.4 Hz, 1H, CHN), 2.82 (m, 1H, CHHNH), 2.99-3.31 (m, 5H, CH₂N×2 & CHHN), 5.25 (s, 4H, CH₂Ar×2), 6.84 (d, J=1.6 Hz, 1H, ArH), 6.89 (d, J=1.6 Hz, 1H, ArH), 7.19-7.38 (m, 9H, ArH); HRMS m/e Cacld. for C₂₆H₂₆Cl₂N₄O₃S₁ 544.1103, found 544.1091.

### Example 73

### 4-Benzyl-6-chloro-2-(2-chloro-benzyl)-8-[(6S)-1,4-diazabicyclo[4.3.0]non-4-yl]-1,1-dioxo-1,4-dihydro-2H-1λ⁶-benzo[1,2,4]thiadiazin-3-one

(yield, 48%), white solid; m.p. 143-144° C.; ¹H NMR (200 MHz, CDC₃) δ 1.28 (m, 1H, CHHCHN), 1.76-1.85 (m, 3H, CHHCHN & CH₂CH₂N), 2.20 (m, 2H, CH N), 2.61 (m, 2H, CH₂N), 2.99-3.10 (m, 3H, CH₂N & CHN), 3.35 (d, J=13.2 Hz, 1H, CHHN), 3.45 (d, J=10.7 Hz, 1H, CHHN), 5.24 (s, 4H, CH₂Ar×2), 6.88 (dd, J=1.5, 24.4 Hz, 2H, ArH), 7.19-7.37 (m, 9H, ArH); HRMS m/e Cacld. for C₂₈H₂₈Cl₂N₄O₃S₁ 570.1259, found 570.1266.

### Example 74

### 4-Benzyl-6-chloro-2-(2-chloro-benzyl)-8-[(6R)-1,4-diazabicyclo[4.3.0]non-4-yl]-1,1-dioxo-1,4-dihydro-2H-1λ⁶-benzo[1,2,4]thiadiazin-3-one

(yield, 46%), white solid; m.p. 114-116° C.; ¹H NMR (200 MHz, CDC₃) δ 1.70-1.92 (m, 3H, CHHCHN & CH₂CH₂N), 2.18-2.35 (m, 3H, CHHCHN & CH₂N), 2.54-2.71 (m, 2H, CH₂N), 2.95-3.14 (m, 3H, CH₂N & CHN), 3.32-3.47 (m, 2H, CH₂N), 5.22-5.27 (m, 4H, NCH₂Ph×2), 6.84 (d, J=1.6 Hz, 1H, ArH), 6.92 (d, J=1.6 Hz, 1H, ArH), 7.19-7.38 (m, 9H, ArH); HRMS m/e Cacld. for C₂₈H₂₈Cl₂N₄O₃S₁ 570.1259, found 570.1259.

### Example 75

### 4-Benzyl-6-chloro-2-(2-chloro-benzyl)-8-[(6R)-1,4-diazabicyclo[4.4.0]dec-4-yl]-1,1-dioxo-1,4-dihydro-2H-1λ⁶-benzo[1,2,4]thiadiazin-3-one

(yield, 35%), white solid; m.p. 157-159° C.; ¹H NMR (200 MHz, CDC₃) δ 1.28 (m, 2H, CH₂CH₂CH₂N), 1.50-1.74 (m, 4H, CH₂CHN & CH₂CH₂N), 2.19 (m, 2H, CH₂N), 2.60 (m, 2H, CH₂N), 2.81 (m, 2H, CH₂N), 3.03 (m, 1H, CHN), 3.17 (d, J=10.8 Hz, 1H, CHHN), 3.32 (d, J=11.4 Hz, 1H, CHHN), 5.25 (s, 4H, CH₂Ar×2), 6.84 (d, J=1.5 Hz, 1H, ArH), 6.89 (d, J=1.8 Hz, 1H, ArH), 7.19-7.37 (m, 9H, ArH); HRMS m/e Cacld. for C₂₉H₃₀Cl₂N₄O₃S₁ 584.1416, found 584.1422.

Structures of compounds prepared by the above examples are listed in Table 1.

TABLE 1

| Example | Structure |
|---|---|
| 1 | 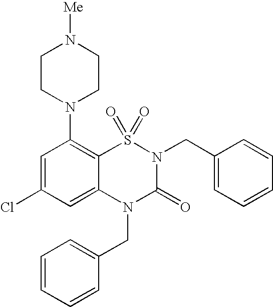 |
| 2 | 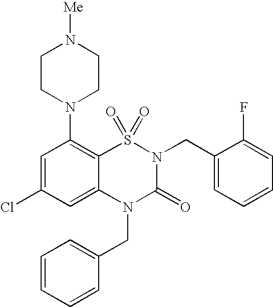 |
| 3 | 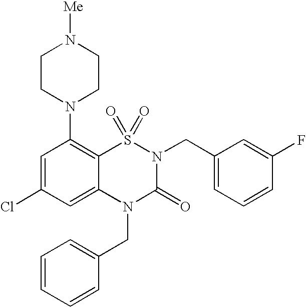 |
| 4 | 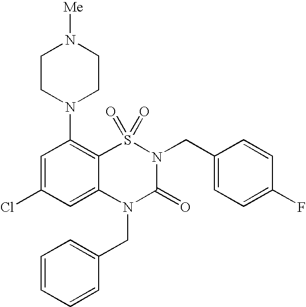 |
| 5 | 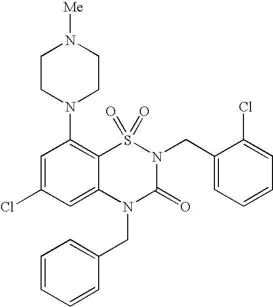 |
| 6 | 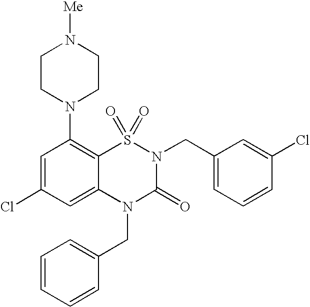 |
| 7 | 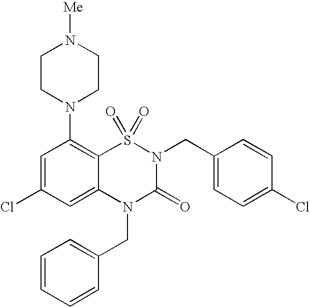 |
| 8 | 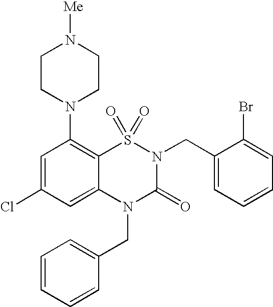 |
| 9 | 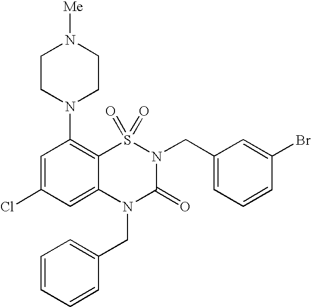 |
| 10 | 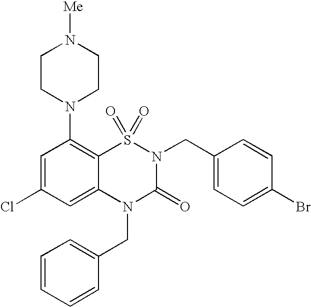 |
| 11 | 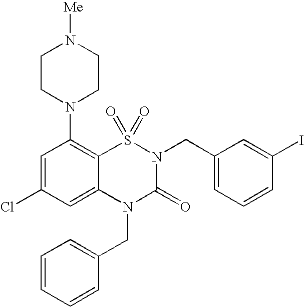 |
| 12 | 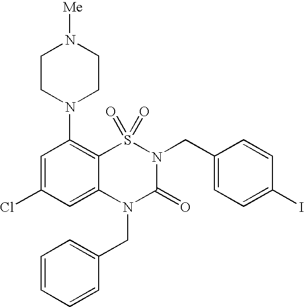 |
| 13 | 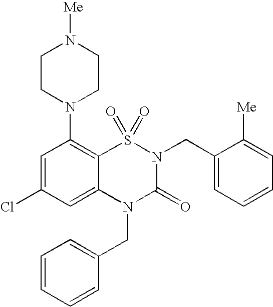 |
| 14 | 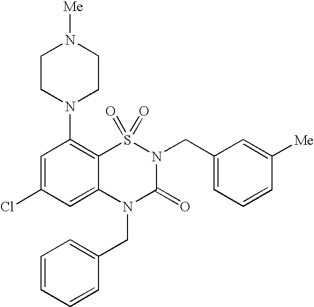 |
| 15 | 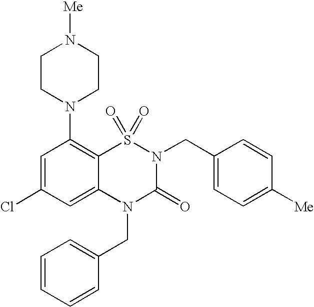 |
| 16 | 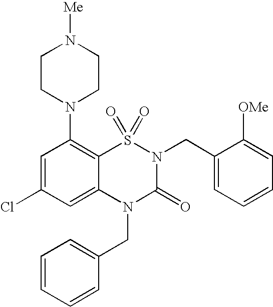 |
| 17 | 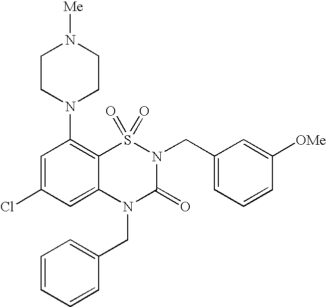 |
| 18 | 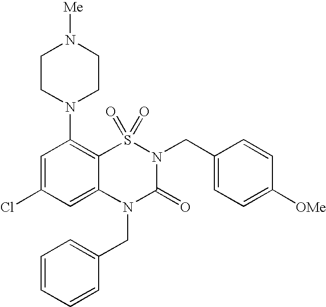 |
| 19 | 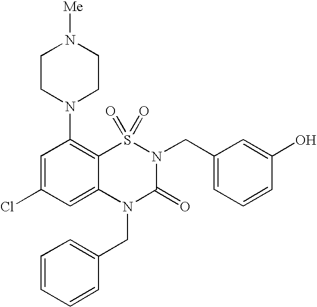 |
| 20 | 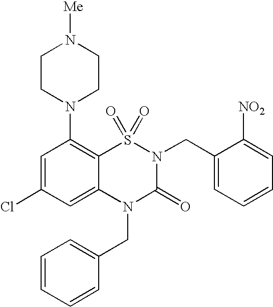 |
| 21 | 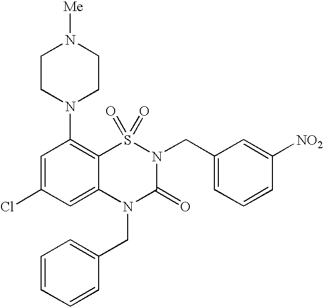 |
| 22 | 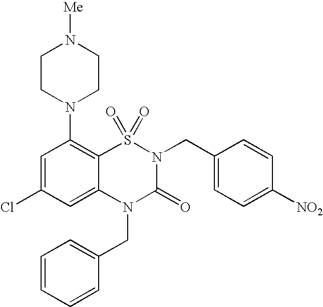 |
| 23 | 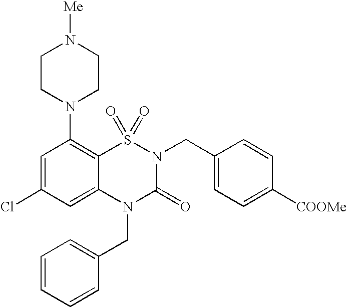 |
| 24 | 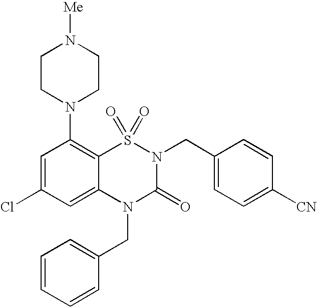 |
| 25 | 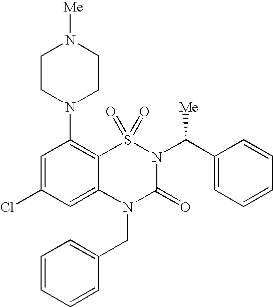 |
| 26 | 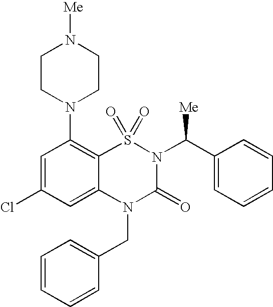 |
| 27 | 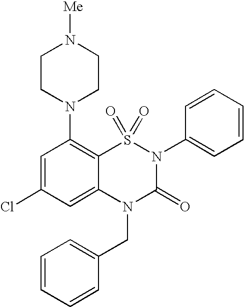 |
| 28 | 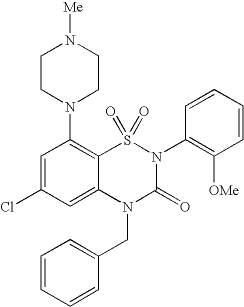 |
| 29 | 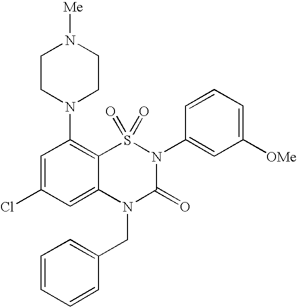 |
| 30 | 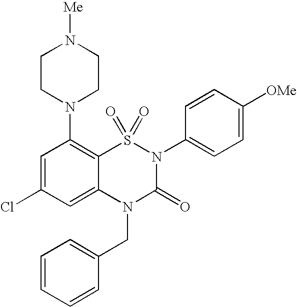 |
| 31 | 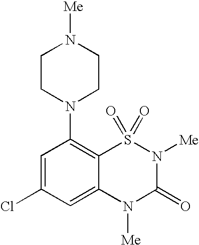 |
| 32 | 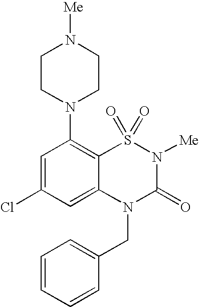 |
| 33 | 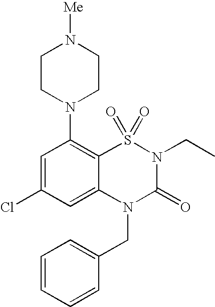 |
| 34 | 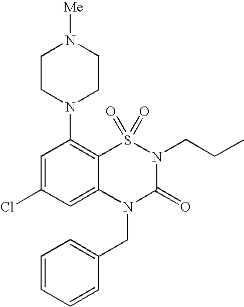 |
| 35 | 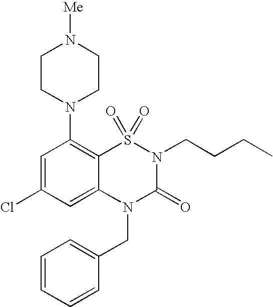 |
| 36 | 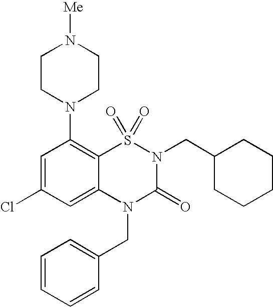 |
| 37 | 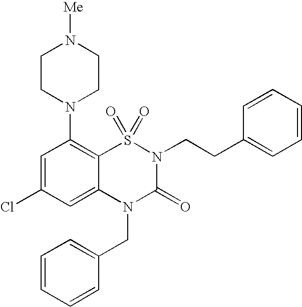 |
| 38 | 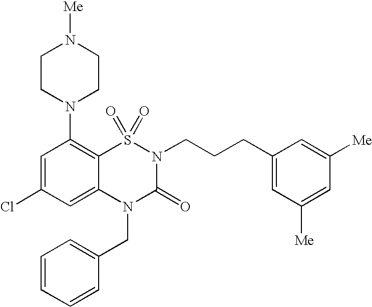 |
| 39 | 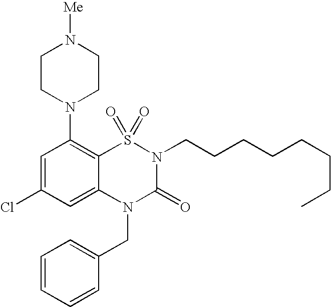 |
| 40 | 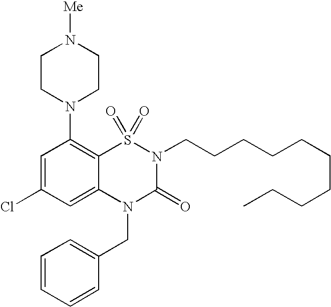 |
| 41 | 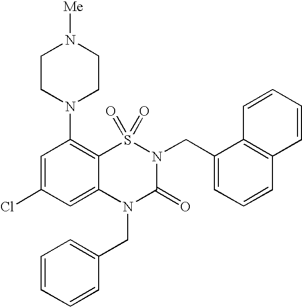 |
| 42 | 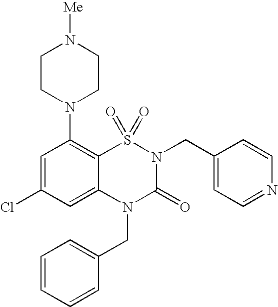 |
| 43 | 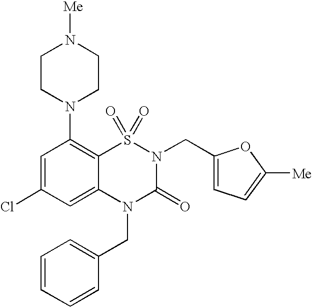 |
| 44 | 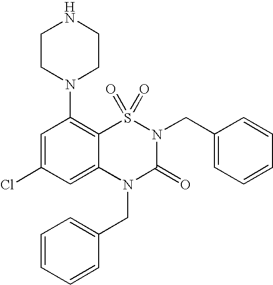 |
| 45 | 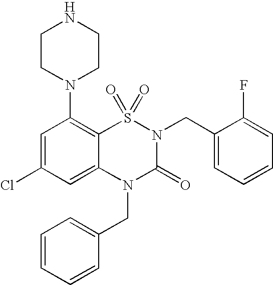 |
| 46 | 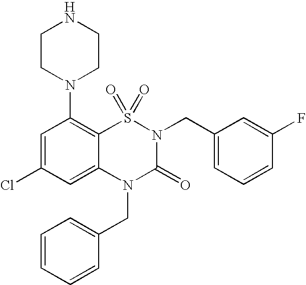 |
| 47 | 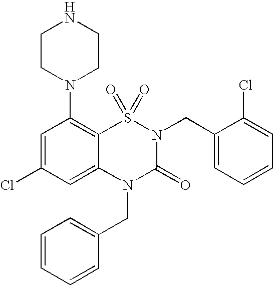 |
| 48 | 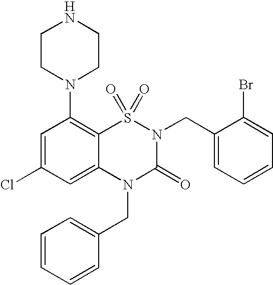 |
| 49 | 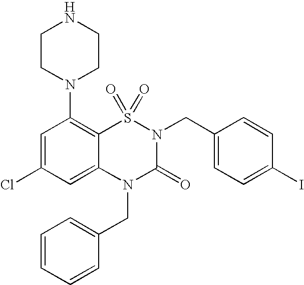 |
| 50 | 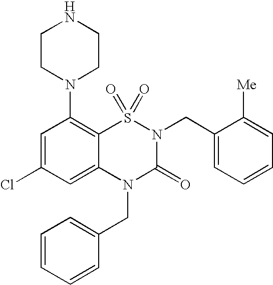 |
| 51 | 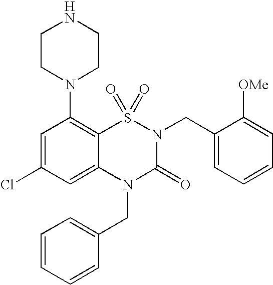 |
| 52 | 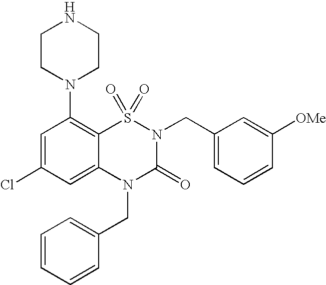 |
| 53 | 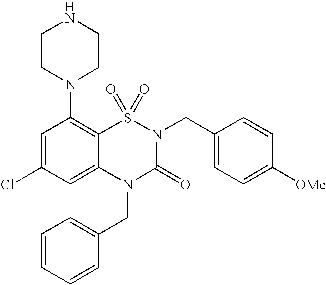 |
| 54 | 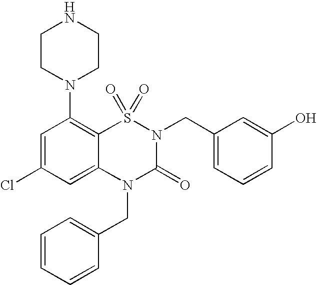 |
| 55 | 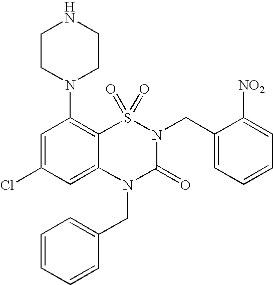 |
| 56 | 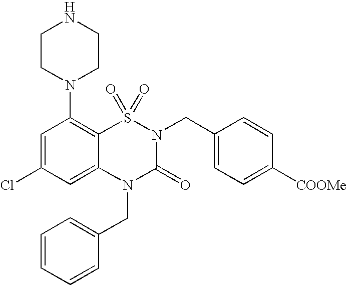 |
| 57 | 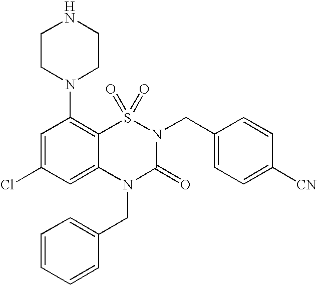 |
| 58 | 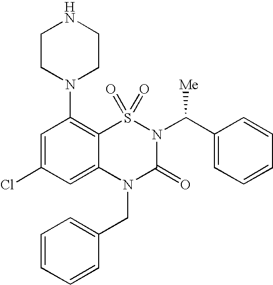 |
| 59 | 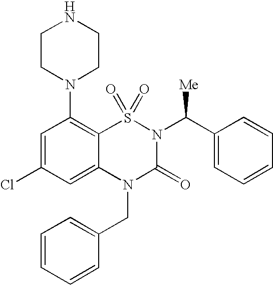 |
| 60 | 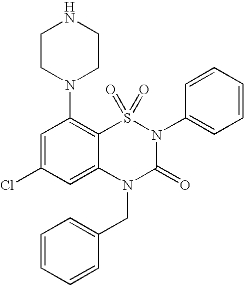 |
| 61 | 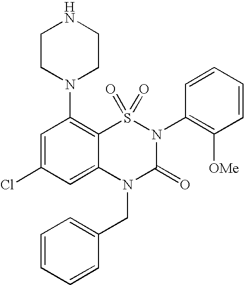 |
| 62 | 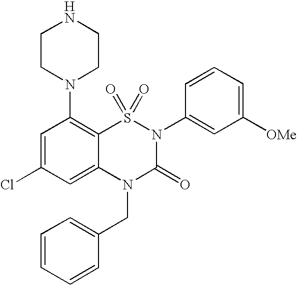 |
| 63 | 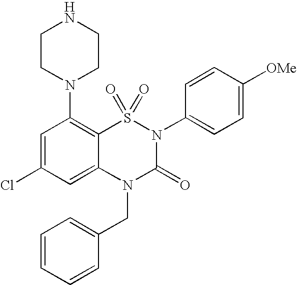 |
| 64 | 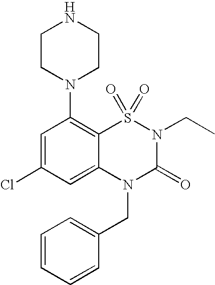 |
| 65 | 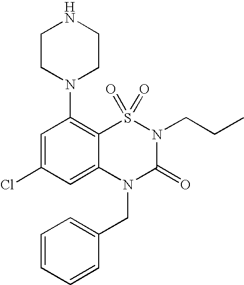 |
| 66 | 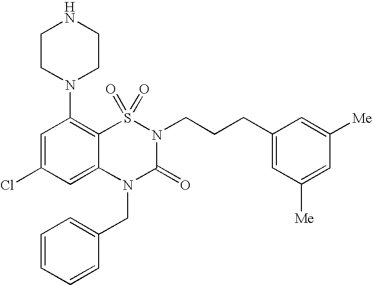 |
| 67 | 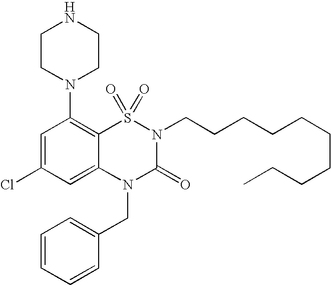 |
| 68 | 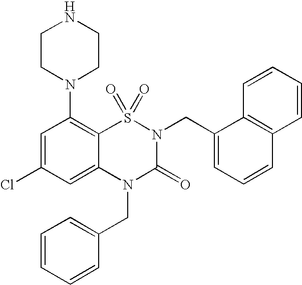 |
| 69 | 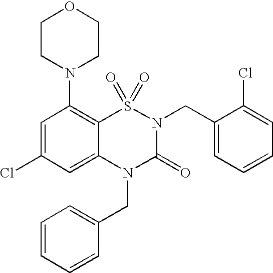 |
| 70 | 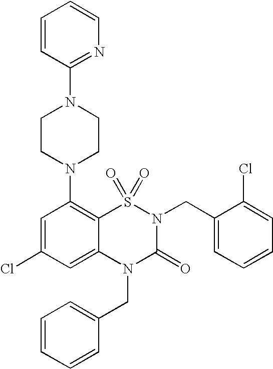 |
| 71 | 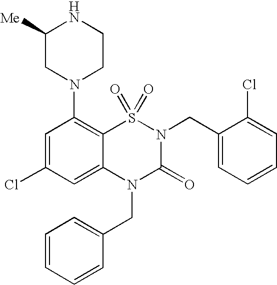 |
| 72 | 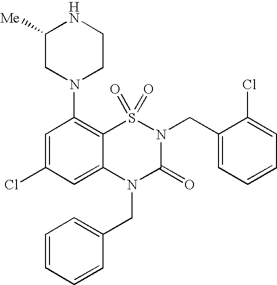 |
| 73 | 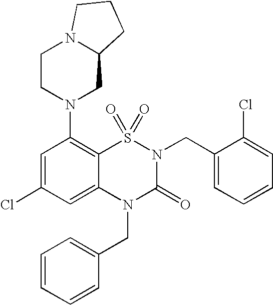 |
| 74 | 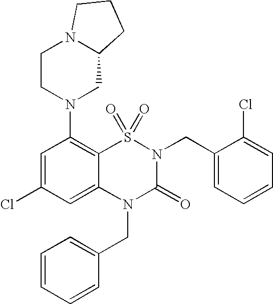 |
| 75 | 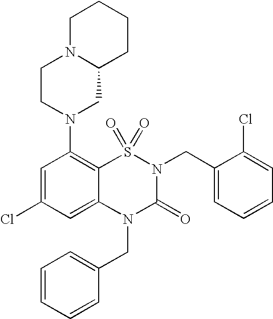 |

### Experimental Example 1

### Binding Affinity of the Compounds According to the Present Invention to 5-HT6 Receptors

1-1: Expression of Human Serotonin 5-HT6 Receptor

Human serotonin 5-HT6 receptor protein was expressed in insect cell as described below. Human 5-HT6 cDNA was cloned from human brain cDNA library (Clontech, Palo Alto, USA) by PCR amplification using 5′-TCATCTGCTTTCCCGCCACCCTAT-3′ for forward and 5′-TCAGGGTCTGGGTTCTGCTCAATC-3′ for reverse. Amplified cDNA fragments were introduced into pGEMT easy vector (Promega, Madison, USA) and then DNA sequencing was performed to confirm receptor DNA sequence. Serotonin 5-HT6 clone was subcloned into insect cell expression vector BacPAK8 (Clontech). pBacPAK8/5-HT6 was transfected into insect Sf21 cell (Clontech) and protein expression of 5-HT6 receptor was confirmed by SDS PAGE and receptor binding assay. Cell lysis was performed by sonication for 2 minutes at 4° C. and cell debris was discarded by centrifugation for 10 min at 3,000×g. Membrane fraction was purified partially from supernatant above by centrifugation for 1 hr at 100,000×g.

1-2: Measurement of Binding Affinity to the Cloned 5-HT6 Receptors

The binding affinity of the compound according to the present invention to 5-HT6 receptor using the cloned 5-HT6 receptor as following.

[³H]LSD (lysergic acid diethylamide) binding assay was performed in 96-well plate to test the binding affinities of the compounds according to the present invention on 5-HT6 receptor. The cloned receptor membranes (9□/well) were used in a final volume of 0.25□ reaction mixture and incubated at 37° C. for 60 min with 50 mM Tris-HCl buffer (pH 7.4) involving 10 mM MgCl₂ and 0.5 mM EDTA. For drug screening, testing compounds were incubated as described above, in a reaction mixture containing 1.87 nM of [³H]LSD. After incubation, the reaction was terminated by the rapid filtration and washed with ice-cold 50 mM Tris-HCl buffer using a Inotech harvester (Inotech, Switzerland) through Wallac GF/C glass fiber filter (Wallac, Finland) which was presoaked in 0.5% PEI. The filter was covered with MeltiLex, sealed in a sample bag followed by drying in the oven, and counted by MicroBeta Plus (Wallac, Finland). Competition binding studies were carried out with 7-8 concentrations of the compound according to the present invention run in duplicate tubes, and isotherms from three assays were calculated by computerized nonlinear regression analysis (GraphPad Prism Program, San Diego, Canada) to yield median inhibitory concentration (IC₅₀) values. Non-specific binding was determined in the presence of 10 μM methiothepin. All testing compounds were dissolved in dimethylsulfoxide (DMSO), and serially diluted to various concentrations for binding assays. 5-HT6 receptor binding affinities of the compounds according to the present invention were shown in Table 2.

TABLE 2
Binding affinity of the compounds according to the present
invention to the 5-HT6 receptor

| Example | IC₅₀ (nM) |
|---|---|
| 1 | 1.9 |
| 2 | 1.9 |
| 3 | 0.7 |
| 4 | 5.4 |
| 5 | 1.3 |
| 6 | 5.2 |
| 7 | 2.3 |
| 8 | 8.9 |
| 9 | 2.2 |
| 10 | 6.1 |
| 11 | 4.4 |
| 12 | 1.4 |
| 13 | 1.6 |
| 14 | 11.6 |
| 15 | 5.4 |
| 16 | 4.6 |
| 17 | 10.6 |
| 18 | 3.6 |
| 19 | 21.3 |
| 20 | 12.3 |
| 21 | 4.2 |
| 22 | 8.1 |
| 23 | 28.8 |
| 24 | 6.4 |
| 25 | 1.1 |
| 26 | 2.4 |
| 27 | 3.8 |
| 28 | 7.1 |
| 29 | 1.7 |
| 30 | 5.2 |
| 31 | 745.3 |
| 32 | 4.2 |
| 33 | 0.3 |
| 34 | 0.4 |
| 35 | 1.9 |
| 36 | 1.5 |
| 37 | 6.9 |
| 38 | 0.6 |
| 39 | 1.8 |
| 40 | 4.5 |
| 41 | 0.8 |
| 42 | 7.7 |
| 43 | 75.9 |
| 44 | 0.9 |
| 45 | 18.7 |
| 46 | — |
| 47 | 15.9 |
| 48 | 46.0 |
| 49 | 17.1 |
| 50 | 22.2 |
| 51 | 48.9 |
| 52 | 5.8 |
| 53 | 11.8 |
| 54 | 1.0 |
| 55 | 33.0 |
| 56 | 8.4 |
| 57 | 18.0 |
| 58 | 2.3 |
| 59 | 3.0 |
| 60 | 15.5 |
| 61 | 50.7 |
| 62 | 10.2 |
| 63 | 28.0 |
| 64 | 4.6 |
| 65 | — |
| 66 | 3.8 |
| 67 | 3.0 |
| 68 | 6.9 |
| 69 | >1000 |
| 70 | >1000 |
| 71 | 147.3 |
| 72 | 6.8 |
| 73 | 16.0 |
| 74 | — |
| 75 | 396.0 |
| methiothepin | 1.2 |

—: Not determined

As shown in the Table 2, the most compounds prepared by Example 1 to 75 had low IC₅₀. Thus, it was confirmed that the compounds of the present invention had good binding affinities at 5-HT6 receptor labeled by [³H]LSD,

### Experimental Example 2

### Radioligand Binding Studies for 5-HT6 Receptorselectivity

The following tests were performed to survey how much the compound showing excellent affinity to 5-HT6 receptor in the above experimental example 1 has selectivity for 5-HT6 receptor, compared to other 5-HT receptors and dopamine receptors.

2-1: Binding Assays of 5-HT Receptor Family

Radioligand bindings were performed according to the test method provided by the supplier of receptor membrane (Euroscreen/BioSignal Packard Inc.). The detailed assay conditions and the results were shown in the following Table 3 and Table 4, respectively.

TABLE 3
Assay condition

| | 5-HT1a | 5-HT2a | 5-HT2c | 5-HT7 |
|---|---|---|---|---|
| Origin | Stable CHO-K1 cell strain expressing human recombinant receptors | | | |
| | (Euroscreen/BioSignal) | | | |
| Binding buffer | 50 mM Tris-HCl(pH 7.4) | 50 mM Tris-HCl(pH 7.4) | 50 mM Tris-HCl(pH 7.7) | 50 mM Tris-HCl(pH 7.4) |
| solution | 10 mM MgSO₄ | | 0.1% ascorbic acid | 10 mM MgSO₄ |
| | 0.5 m MEDTA | | 10 μM Pargyline | 0.5 mM EDTA |
| | 0.1% ascorbic acid | | | |
| Final volume | 250 □ | 250 □ | 250 □ | 250 □ |
| Membrane | 40 □ | 15 □ | 4 □ | 10 □ |
| content | | | | |
| Radioligand | [³H]8-OH-DPAT | [³H]Ketanserin | [³H]Mesulergine | [³H] LSD 3 nM |
| | 0.5 nM | 1 nM | 1 nM | |
| Non-specific | methiothepin 0.5 M | Mianserin 1 M | methiothepin 10 M | methiothepin 10 M |
| Binding | | | | |
| Incubation | 27° C., 60 min | 37° C., 15 min | 37° C., 30 min | 27° C., 120 min |
| Filtration | GF/C, 0.3% PEI | GF/C, 0.05% Brij | GF/C, 1% BSA | GF/C, 0.3% PEI |

2-2: Binding Assays of Dopamine Receptor Family

The radioligands used were [³H]spiperone (for hD_{2L} and hD₃ receptors, 1 nM) and [³H] YM-09151-2 (for hD_{4.2} receptor, 0.06 nM). Radioligand bindings were performed by the protocols provided by the supplier of receptor membranes (BioSignal Packard Inc., Montreal, Canada). Briefly, the buffer used in D₂ or D₃ receptor binding assay was 50 mM Tris-HCl (pH 7.4), 10 mM MgCl₂, 1 mM EDTA, or 50 mM Tris-HCl (pH 7.4), 5 mM MgCl₂ 5 mM EDTA, 5 mM KCl, 1.5 mM CaCl₂, 120 mM NaCl, respectively. In [³H] YM-09151-2 receptor binding assays, the buffer containing 50 mM Tris-HCl (pH 7.4), 5 mM MgCl₂, 5 mM EDTA, 5 mM KCl and 1.5 mM CaCl₂ was used. Nonspecific binding was determined with haloperidol (10 μM) or clozapine (10 μM) for D₂ and D₃, and D₄ receptors, respectively. Competition binding studies were carried out with 7-8 concentrations of the test compound run in duplicate tubes, and isotherms from three assays were calculated by computerized nonlinear regression analysis (GraphPad Prism Program, San Diego, Canada) to yield median inhibitory concentration (IC₅₀) values.

The other serotonin receptor subtypes and dopamine selectivity of compounds according to the present invention was shown in Table 4.

TABLE 4
The other serotonin receptor subtypes and dopamine selectivity of compounds
according to the present invention

| | Binding affinity, IC₅₀ (nM) | | | | | | | | |
|---|---|---|---|---|---|---|---|---|---|
| Example | 5-HT6 | 5-HT1a | 5-HT2a | 5-HT2c | 5-HT7 | D1 | D2 | D3 | D4 |
| 1 | 1.9 | 997 | 354 | 785 | 6583 | 3054 | 5807 | 633 | >10000 |
| 2 | 1.9 | 2300 | 335 | 578 | >10000 | — | >10000 | 390 | >10000 |
| 3 | 0.7 | 5979 | 846 | 2186 | >10000 | — | >10000 | 537 | >10000 |
| 5 | 1.3 | 3005 | 396 | 240 | >10000 | — | 5090 | 93 | >10000 |
| 7 | 2.3 | 3911 | 488 | 1539 | >10000 | — | >10000 | 555 | >10000 |
| 17 | 10.9 | 1955 | 318 | 899 | >10000 | 4734 | 4865 | 537 | >10000 |
| 18 | 3.6 | 3417 | 563 | 990 | >10000 | >10000 | 6583 | 1216 | >10000 |
| 25 | 1.1 | >10000 | 350 | 691 | 8560 | 963 | 1923 | 265 | >10000 |
| 26 | 2.4 | 589 | 145 | 183 | 8252 | 1760 | 6583 | 715 | >10000 |
| 27 | 3.8 | 2084 | 4313 | 2576 | >10000 | 2688 | >10000 | 5954 | >10000 |
| 33 | 0.3 | 2616 | 119 | 1242 | >10000 | — | >10000 | 3168 | >10000 |
| 34 | 0.4 | 2393 | 436 | 203 | >10000 | — | >10000 | 1864 | >10000 |
| 38 | 0.6 | 3417 | 359 | 508 | 9138 | 918 | >10000 | 336 | >10000 |
| 39 | 1.8 | 894 | 1321 | 1051 | 3173 | — | >10000 | 130 | >10000 |
| 41 | 0.8 | >10000 | 365 | 257 | >10000 | — | >10000 | 420 | >10000 |
| 44 | 1.0 | 5399 | 1160 | 1882 | >10000 | 6392 | 667 | 1209 | >10000 |
| 52 | 2.0 | 1861 | 1003 | 1351 | 6784 | 3137 | 3216 | 531 | >10000 |
| 53 | 11.8 | 3255 | 651 | 961 | 6583 | >10000 | 2440 | 844 | >10000 |
| 54 | 5.8 | 621 | 1154 | 1795 | >10000 | 4105 | 2277 | 804 | >10000 |
| 58 | 2.3 | >10000 | 549 | 424 | 6392 | 1779 | 4497 | 398 | >10000 |
| 59 | 3.0 | 1403 | 272 | 508 | 2301 | 4099 | 4645 | 403 | >10000 |
| 62 | 10.2 | 926 | 3165 | 4959 | >10000 | 5592 | >10000 | 2368 | >10000 |
| 66 | 3.8 | 1836 | 710 | 498 | 4533 | 1183 | 1643 | 488 | >10000 |
| SB-271046 | 0.8 | 313 | 4651 | 3963 | 3498 | 9138 | >10000 | 4119 | >10000 |

—: Not determined

As shown in Table 4, the compounds according to the present invention had much lower IC levels for 5-HT6 receptor than other 5-HT receptors and dopamine receptors, and it was confirmed that the compounds had very excellent binding affinities to 5-HT6 receptor compared to other 5-HT receptors and other family receptors.

### Experimental Example 3

### In Vitro Functional Studies

By a method (2000) disclosed by Rutledge et al. of MDS Pharma Service (Bothell, Wash., USA, MDSPS PT #1037161), activity of adenylil cyclase in HeLa cell having transfected with human 5-HT6 receptor was measured.

Details of the assay conditions were shown in Table 5. The assay mixture consisted of Hanks' balanced salt solution (HBSS, pH 7.4) containing: 1 mM MgCl₂, 1 mM CaCl₂, 100 mM 1-methyl-3-isobutylxanthine. Incubation was started by addition of membrane suspension and compounds according to the present invention. Following the a 20 minutes incubation at 37° C., intracellular cAMP levels were measured by EIA (enzyme-immunoassay), and a compound showing inhibitory effects on serotonin(5-HT)-stimulated cAMP accumulation was classified into an antagonist. And methiothepin was used as reference 5-HT antagonist for comparison.

TABLE 5
Assay conditions of adenylyl cyclase activity in
HeLa cells transfected with human 5-HT6 receptor

| | |
|---|---|
| Target | Human HeLa cells |
| Vehicle | 0.4% DMSO |
| Incubation time/temp | 20 min at 37° C. |
| Incubation buffer | HBSS (pH. 7.4), 1 mM MgCl₂, 1 mM CaCl₂, |
| | 100 mM IBMX |
| Quantitation method | EIA quantitation of cAMP accumulation |
| Significance criteria- | ≧50% inhibition of serotonin (0.3 μM)- |
| Antagonist | induced cAMP increase |
| Significance criteria- | ≧50% increase in cAMP relative to serotonin |
| Agonist | response |

The results were shown in FIG. 1.

As shown in FIG. 1, the human 5-HT6 receptor showed a 5-HT concentration-dependent increase in cAMP levels with an EC₅₀=16.9 nM, and this increase in cAMP level was inhibited by Example 44 or methiothepin, a reference 5-HT6 antagonist. Particularly, Example 44 of 0.001, 0.01, 0.1, 1 and 10 μM potently inhibited the 0.3 μM serotonin (5-HT)-induced increase in cAMP levels by 0, 8, 63, 100 and 100%, respectively. And the IC₅₀ of Example 44 was 67.8 nM, which was demonstrating significant antagonist activity. In addition, Example 44 did not show any cytotoxicity at the concentrations tested in HeLa cells transfected with the human 5-HT6 receptor.

### Experimental Example 4

### In Vivo Study of the Effect on Apomorphine-Induced Disruption of Prepulse Inhibition (PPI) in Rats

To assay antipsychotic properties of the compounds according to the invention, prepulse inhibition (PPI) of acoustic startle in animals was performed.

Startle response was measured using SR-LAB startle chamber (San Diego Instruments, San Diego, USA).

The animal enclosure was housed in a ventilated and sound-attenuated startle chamber with 60 dB ambient noise level, and consisted of a Plexiglas cylinder 40 □ in diameter on a platform, connected to a piezoelectric accelerometer which detects and transducer motion within the cylinder. Acoustic noise bursts were presented through a loudspeaker mounted 24□ above the animal.

Behavioral testing was performed between 10 a.m. and 5 p.m., during the light phase by a modified Mansbach et al's method [Mansbach R S, Brooks E W, Sanner M A, Zorn S H, Selective dopamine D4 receptor antagonists reverse apomorphine-induced blockade of prepulse inhibition, Psychopharmacology (Berl), 135:194-200, 1998]. Each startle session began with a 5-min acclimatization period in the chamber to 68 dB background noises. The test session consisting of the following four different trial types was carried for all experiments: a 40 ms broadband 120 dB burst (P; pulse alone trial), P preceded 100 ms earlier by a 20 ms noise burst 10 dB above background (pP; prepulse+pulse trial), a 40 ms broadband 78 dB burst (prepulse alone trial), and a no stimulus trial (background). Eight trials of each type were presented in a pseudorandom order (total 32 trials) with an average interval of 15 sec. separating each trial. An extra 5 pulse-alone trials were presented at the beginning and end of each test session, but were not used in the calculation of PPI values. PPI was defined as the percent reduction in startle amplitude in the presence of prepulse compared to the amplitude in the absence of the prepulse using the following Math Equation 1.

PPI (%)=[100−(100×startle amplitude on pP trial/startle amplitude on P trial)] <Math Equation 1>

The rats were administered (i.p.) with either Example 1, Example 44, SB-271046 or vehicle, 30 min before the injection of apomorphine (2 mg/kg, i.p.), and were placed in the startle chamber 30 min after the apomorphine injection for testing. Example 1, 44 and SB-271046 were suspended in 3% Tween 80 solution.

Statistical significance of the results was evaluated by one-way analysis of variance (ANOVA) with Dunnett's post-hoc tests for comparing control to treatment. Differences were considered significant at P<0.05. Statistical analyses were conducted using SigmaStat software (SigmaStat, Jandel Co., San Rafael, Calif.). The data were expressed as means±SEM.

The results were shown in FIG. 2.

As shown in FIG. 2, the Example 1 (2.5, 5, 10 & 20 mg/kg, i.p.) or Example 44 (2.5, 5, 10 & 20 mg/kg, i.p.) alone had no significant effect on PPI when compared to vehicle control in rats. However, the disruption of PPI by apomorphine (2 mg/kg, i.p.) was reversed significantly by pretreatment with Example 1 (P=0.038) and 44 (P=0.014) (FIG. 3), indicating significant antipsychotic activity. There were no significant differences in mean startle amplitude of Example 1 and Example 44 administered 30 min before apomorphine when compared with that of apomorphine control group. However, SB-271046 (2.5, 5, 10 and 20 mg/kg, ip) did not reverse the disruption of PPI induced by apomorphine.

### Experimental Example 5

### Effect on Rotarod Deficit in Mice

The mouse was placed on a 1 inch diameter knurled plastic rod rotating at 6 rpm (Ugo-Basile, Milano, Italy), and the rotarod deficit (%) was obtained by counting the number of animals fallen from the rotating rod within 1 min (Dunham et al., 1957) at 30, 60, 90 and 120 min after the injection of test compound. The median neurotoxic dose (TD₅₀) was determined as the dose at which 50% of animals showed rotarod deficit. Example 1, 44 or SB-271046 was suspended in 3% Tween 80 solution, and was administered (ip) 30 min before the testing.

The result was shown in Table 6.

TABLE 6
Effect on rotarod deficit in mice of the compounds
according to the present invention

| Example | rotarod deficit TD₅₀ (□/□, p.o.) |
|---|---|
| 1 | >>300 |
| 44 | 161 |
| SB-271046 | 112 |

As shown in Table 5, a single administration of Example 1 did not show any rotarod ataxia at the doses up to 300 □/□ for 120 min after the treatment. Thus, their median neurotoxic dose (TD₅₀) was calculated to more than 300 □/□ (ip) in mice, demonstrating that Example 1 has much lower liability to induce extrapyramidal side effects than SB-271046, which showed 112 □/□ of TDvalue. Meanwhile, Example 44 produced moderate rotarod deficit showing 161 □/□ of TD50 value. However, this TD value is about 8 times higher than effective doses obtained in the PPI test, indicating Example 44 may also be a relatively safe drug.

### Formulation Example 1

### Pharmaceutical Formulations

1-1. Preparation of Powder

The compound according to the present invention, a pharmaceutically acceptable salt or a prodrug thereof 2 g

Lactose 1 g

Powder product was prepared by mixing the above ingredients and filling an airtight package therewith.

1-2. Preparation of Tablet

The compound according to the present invention, a pharmaceutically acceptable salt or a prodrug thereof 100 □

Corn starch 100 □

Lactose 100 □

Magnesium stearate 2 □

Tablets were prepared by mixing the above ingredients and tabletting by a conventional method.

1-3. Preparation of Capsule

The compound according to the present invention, a pharmaceutically acceptable salt or a prodrug thereof 100 □

Corn starch 100 □

Lactose 100 □

Magnesium stearate 2 □

Capsules were prepared by mixing the above ingredients and filling a gelatin capsule by a conventional method.

However, the Formulation containing the compounds of Formula 1 according to the present invention are not limited to the above-listed compounds.

### INDUSTRIAL APPLICABILITY

The compounds of substituted-1,1-dioxo-benzo[1,2,4]thiadiazin-3-ones according to the present invention have excellent binding affinity to the 5HT6 receptor, excellent selectivity for the 5HT6 receptor over other receptors, the inhibitory effect of the serotonin(5-HT)-stimulated cAMP accumulation and an effect on apomorphine(2 □/□, i.p.)-induced disruption of prepulse inhibition (PPI) in rats. Also, the compounds of the present invention of effective dose don't show any rotarod deficits in mice. Therefore, The compounds of substituted-1,1-dioxo-benzo[1,2,4]thiadiazin-3-ones according to the present invention may be useful to composition for treatment of a 5HT6 receptor relating disorders such as cognitive disorders, Alzheimers disease, anxiety, depression, schizophrenia, stress disorder, panic disorder, phobic disorder, obsessive compulsive disorder, post traumatic stress disorder, immune system depression, psychosis, paraphrenia, mania, convulsive disorder, personality disorder, migraine, drug addiction, alcoholism, obesity, eating disorder, and sleep disorder.

1. A compound of substituted-1,1-dioxo-benzo[1,2,4]thiadiazin-3-one represented by the following formula 1 or a pharmaceutically acceptable salt thereof: 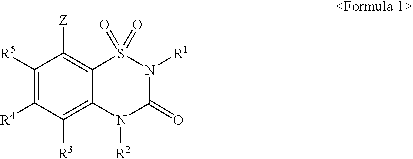
wherein,
R¹ represents hydrogen, C₁˜C₁₀ alkyl, C₃˜C₁₀ aryl, C₃˜C₇ cycloalkyl, arylalkyl, heteroaryl, or heteroarylalkyl;
R² represents hydrogen, C₁˜C₁₀ alkyl, C₃˜C₁₀ aryl, heteroaryl, aralkyl, heteroarylalkyl, amino or cyclic amino;
R³, R⁴ and R⁵ independently represent hydrogen, halogen, amino, cyclic amino, nitro, cyano, C₁˜C₁₀ alkyl, haloalkyl, C₁˜C₇ alkoxy, haloalkoxy, piperazinyl or N-methyl piperazinyl; and
Z represents saturated mono-, bi-, or tri-cyclic amines containing 1 to 3 nitrogen atoms and 5 to 12 carbon atoms in the ring.

2. The compound of substituted-1,1-dioxo-benzo[1,2,4]thiadiazin-3-one or a pharmaceutically acceptable salt thereof of according to claim 1, wherein,
R¹ represents C₁˜C₁₀ alkyl, C₃˜C₇ cycloalkyl, phenyl, benzyl, naphtalenyl, pyridinyl, furanyl C₃˜C₇ cycloalkyl substituted by one or two substituent selected from the group consisting of (C₁˜C₄) alkyl, (C₁˜C₄) alkoxy, halogen, nitro, amino, cyano, hydroxy and methyl carboxylate, phenyl substituted by one or two substituent selected from the group consisting of (C₁˜C₄) alkyl, (C₁˜C₄) alkoxy, halogen, nitro, amino, cyano hydroxy and methyl carboxylate, benzyl substituted by one or two substituent selected from the group consisting of (C₁˜C₄) alkyl, (C₁˜C₄) alkoxy, halogen, nitro, amino, cyano, hydroxy and methyl carboxylate, naphtalenyl substituted by one or two substituent selected from the group consisting of (C₁˜C₄) alkyl, (C₁˜C₄) alkoxy, halogen, nitro, amino, cyano, hydroxy and methyl carboxylate, pyridinyl substituted by one or two substituent selected from the group consisting of (C₁˜C₄) alkyl, (C₁˜C₄) alkoxy, halogen, nitro, amino, cyano, hydroxy and methyl carboxylate or furanyl substituted by one or two substituent selected from the group consisting of (C₁˜C₄) alkyl, (C₁˜C₄) alkoxy, halogen, nitro, amino, cyano, hydroxy and methyl;
R² represents C₁˜C₄ alkyl, phenyl or benzyl;
R³, R⁴ and R⁵ independently represent hydrogen, halogen or methoxy; and
Z represents piperazinyl, piperazinyl substituted by C₁˜C₄ alkyl or amine, morpholinyl, pyrrolyl, pyridinyl or diazabicycloalkyl.

3. The compound of substituted-1,1-dioxo-benzo[1,2,4]thiadiazin-3-one or a pharmaceutically acceptable salt thereof according to claim 2, wherein,
R¹ represents methyl, ethyl, propyl, n-butyl, octyl, decyl; phenyl, benzyl, furanyl; cyclohexylmethyl, phenethyl, (R)-1-phenyl-ethyl, (S)-1-phenyl-ethyl, phenylpropyl, methoxyphenyl, dimethylphenylpropyl, fluorobenzyl, chlorobenzyl, bromobenzyl, methylbenzyl, methoxybenzyl, iodobenzyl, hydroxybenzyl, nitrobenzyl, cyanobenzyl, methyl carboxylatebenzyl, naphtalenylmethyl or pyridinylmethyl;
R² represents benzyl;
R³, R⁴ and R⁵ independently represent hydrogen, chlorine, bromine or methoxy; and
Z represents piperazinyl, methylpiperazinyl, pyridinyl-piperazinyl, morpholinyl, diazabicyclononyl, diazabicyclodecyl or diazabicyclooctyl.

4. The compound of substituted-1,1-dioxo-benzo[1,2,4]thiadiazin-3-one or a pharmaceutically acceptable salt thereof according to claim 3 selected from the group consisting of:
(1) 2,4-Dibenzyl-6-chloro-8-(4-methyl-piperazin-1-yl)-1,1-dioxo-1,4-dihydro-2H-1λ⁶-benzo[1,2,4]-thiadiazin-3-one,
(2) 4-Benzyl-6-chloro-2-(2-fluoro-benzyl)-8-(4-methyl-piperazin-1-yl)-1,1-dioxo-1,4-dihydro-2H-1λ⁶-benzo[1,2,4]thiadiazin-3-one,
(3) 4-Benzyl-6-chloro-2-(3-fluoro-benzyl)-8-(4-methyl-piperazin-1-yl)-1,1-dioxo-1,4-dihydro-2H-1λ⁶-benzo[1,2,4]thiadiazin-3-one,
(4) 4-Benzyl-6-chloro-2-(4-fluoro-benzyl)-8-(4-methyl-piperazin-1-yl)-1,1-dioxo-1,4-dihydro-2H-1λ⁶-benzo[1,2,4]thiadiazin-3-one,
(5) 4-Benzyl-6-chloro-2-(2-chloro-benzyl)-8-(4-methyl-piperazin-1-yl)-1,1-dioxo-1,4-dihydro-2H-1λ⁶-benzo[1,2,4]thiadiazin-3-one,
(6) 4-Benzyl-6-chloro-2-(3-chloro-benzyl)-8-(4-methyl-piperazin-1-yl)-1,1-dioxo-1,4-dihydro-2H-1λ⁶-benzo[1,2,4]thiadiazin-3-one,
(7) 4-Benzyl-6-chloro-2-(4-chloro-benzyl)-8-(4-methyl-piperazin-1-yl)-1,1-dioxo-1,4-dihydro-2H-1λ⁶-benzo[1,2,4]thiadiazin-3-one,
(8) 4-Benzyl-2-(2-bromo-benzyl)-6-chloro-8-(4-methyl-piperazin-1-yl)-1,1-dioxo-1,4-dihydro-2H-1λ⁶-benzo[1,2,4]thiadiazin-3-one,
(9) 4-Benzyl-2-(3-bromo-benzyl)-6-chloro-8-(4-methyl-piperazin-1-yl)-1,1-dioxo-1,4-dihydro-2H-1λ⁶-benzo[1,2,4]thiadiazin-3-one,
(10) 4-Benzyl-2-(4-bromo-benzyl)-6-chloro-8-(4-methyl-piperazin-1-yl)-1,1-dioxo-1,4-dihydro-2H-1λ⁶-benzo[1,2,4]thiadiazin-3-one,
(11) 4-Benzyl-6-chloro-2-(3-iodo-benzyl)-8-(4-methyl-piperazin-1-yl)-1,1-dioxo-1,4-dihydro-2H-1λ⁶-benzo[1,2,4]thiadiazin-3-one,
(12) 4-Benzyl-6-chloro-2-(4-iodo-benzyl)-8-(4-methyl-piperazin-1-yl)-1,1-dioxo-1,4-dihydro-2H-1λ⁶-benzo[1,2,4]thiadiazin-3-one,
(13) 4-Benzyl-6-chloro-2-(2-methyl-benzyl)-8-(4-methyl-piperazin-1-yl)-1,1-dioxo-1,4-dihydro-2H-1λ⁶-benzo[1,2,4]thiadiazin-3-one,
(14) 4-Benzyl-6-chloro-2-(3-methyl-benzyl)-8-(4-methyl-piperazin-1-yl)-1,1-dioxo-1,4-dihydro-2H-1λ⁶-benzo[1,2,4]thiadiazin-3-one,
(15) 4-Benzyl-6-chloro-2-(4-methyl-benzyl)-8-(4-methyl-piperazin-1-yl)-1,1-dioxo-1,4-dihydro-2H-1λ⁶-benzo[1,2,4]thiadiazin-3-one,
(16) 4-Benzyl-6-chloro-2-(2-methoxy-benzyl)-8-(4-methyl-piperazin-1-yl)-1,1-dioxo-1,4-dihydro-2H-1λ⁶-benzo[1,2,4]thiadiazin-3-one,
(17) 4-Benzyl-6-chloro-2-(3-methoxy-benzyl)-8-(4-methyl-piperazin-1-yl)-1,1-dioxo-1,4-dihydro-2H-1λ⁶-benzo[1,2,4]thiadiazin-3-one,
(18) 4-Benzyl-6-chloro-2-(4-methoxy-benzyl)-8-(4-methyl-piperazin-1-yl)-1,1-dioxo-1,4-dihydro-2H-1λ⁶-benzo[1,2,4]thiadiazin-3-one,
(19) 4-Benzyl-8-chloro-2-(3-hydroxy-benzyl)-6-(4-methyl-piperazin-1-yl)-1,1-dioxo-1,4-dihydro-2H-1λ⁶-benzo[1,2,4]thiadiazin-3-one,
(20) 4-Benzyl-6-chloro-8-(4-methyl-piperazin-1-yl)-2-(2-nitro-benzyl)-1,1-dioxo-1,4-dihydro-2H-1λ⁶-benzo[1,2,4]thiadiazin-3-one,
(21) 4-Benzyl-6-chloro-8-(4-methyl-piperazin-1-yl)-2-(3-nitro-benzyl)-1,1-dioxo-1,4-dihydro-2H-1λ⁶-benzo[1,2,4]thiadiazin-3-one,
(22) 4-Benzyl-6-chloro-8-(4-methyl-piperazin-1-yl)-2-(4-nitro-benzyl)-1,1-dioxo-1,4-dihydro-2H-1λ⁶-benzo[1,2,4]thiadiazin-3-one,
(23) 4-[4-Benzyl-6-chloro-8-(4-methyl-piperazin-1-yl)-1,1,3-trioxo-3,4-dihydro-1H-1λ⁶-benzo[1,2,4]thiadiazin-2-ylmethyl]-benzoic acid methyl ester,
(24) 4-[4-Benzyl-6-chloro-8-(4-methyl-piperazin-1-yl)-1,1,3-trioxo-3,4-dihydro-2H-1λ⁶-benzo[1,2,4]thiadiazin-2-ylmethyl]-benzonitrile,
(25) 4-Benzyl-6-chloro-8-(4-methyl-piperazin-1-yl)-1,1-dioxo-2-[(R)-1-phenyl-ethyl]-1,4-dihydro-2H-1λ⁶-benzo[1,2,4]thiadiazin-3-one,
(26) 4-Benzyl-6-chloro-8-(4-methyl-piperazin-1-yl)-1,1-dioxo-2-[(S)-1-phenyl-ethyl]-1,4-dihydro-2H-1λ⁶-benzo[1,2,4]thiadiazin-3-one,
(27) 4-Benzyl-6-chloro-8-(4-methyl-piperazin-1-yl)-1,1-dioxo-2-phenyl-1,4-dihydro-2H-1λ⁶-benzo[1,2,4]thiadiazin-3-one,
(28) 4-Benzyl-6-chloro-2-(2-methoxy-phenyl)-8-(4-methyl-piperazin-1-yl-1,1-dioxo-1,4-dihydro-2H-1λ⁶-benzo[1,2,4]thiadiazin-3-one,
(29) 4-Benzyl-6-chloro-2-(3-methoxy-phenyl)-8-(4-methyl-piperazin-1-yl)-1,1-dioxo-1,4-dihydro-2H-1λ⁶-benzo[1,2,4]thiadiazin-3-one,
(30) 4-Benzyl-6-chloro-2-(4-methoxy-phenyl)-8-(4-methyl-piperazin-1-yl)-1,1-dioxo-1,4-dihydro-2H-1λ⁶-benzo[1,2,4]thiadiazin-3-one,
(31) 6-Chloro-2,4-dimethyl-8-(4-methyl-piperazin-1-yl)-1,1-dioxo-1,4-dihydro-2H-1λ⁶-benzo[1,2,4]thiadiazin-3-one,
(32) 4-Benzyl-6-chloro-2-methyl-8-(4-methyl-piperazin-1-yl)-1,1-dioxo-1,4-dihydro-2H-1λ⁶-benzo[1,2,4]thiadiazin-3-one,
(33) 4-Benzyl-6-chloro-2-ethyl-8-(4-methyl-piperazin-1-yl)-1,1-dioxo-1,4-dihydro-2H-1λ⁶-benzo[1,2,4]thiadiazin-3-one,
(34) 4-Benzyl-6-chloro-8-(4-methyl-piperazin-1-yl)-2-propyl-1,1-dioxo-1,4-dihydro-2H-1λ⁶-benzo[1,2,4]thiadiazin-3-one,
(35) 4-Benzyl-2-butyl-6-chloro-8-(4-methyl-piperazin-1-yl)-1,1-dioxo-1,4-dihydro-2H-1λ⁶-benzo[1,2,4]thiadiazin-3-one,
(36) 4-Benzyl-6-chloro-2-cyclohexylmethyl-8-(4-methyl-piperazin-1-yl)-1,1-dioxo-1,4-dihydro-2H-1λ⁶-benzo[1,2,4]thiadiazin-3-one,
(37) 4-Benzyl-6-chloro-8-(4-methyl-piperazin-1-yl)-1,1-dioxo-2-phenethyl-1,4-dihydro-2H-1-benzo[1,2,4]thiadiazin-3-one,
(38) 4-Benzyl-6-chloro-2-[3-(3,5-dimethyl-phenyl)-propyl]-8-(4-methyl-piperazin-1-yl)-1,1-dioxo-1,4-dihydro-2H-1λ⁶-benzo[1,2,4]thiadiazin-3-one,
(39) 4-Benzyl-6-chloro-8-(4-methyl-piperazin-1-yl)-2-octyl-1,1-dioxo-1,4-dihydro-2H-1λ⁶-benzo[1,2,4]thiadiazin-3-one,
(40) 4-Benzyl-6-chloro-2-decyl-8-(4-methyl-piperazin-1-yl)-1,1-dioxo-1,4-dihydro-2H-1λ⁶-benzo[1,2,4]thiadiazin-3-one,
(41) 4-Benzyl-6-chloro-8-(4-methyl-piperazin-1-yl)-2-naphthalen-1-ylmethyl-1,1-dioxo-1,4-dihydro-2H-1λ⁶-benzo[1,2,4]thiadiazin-3-one,
(42) 4-Benzyl-6-chloro-8-(4-methyl-piperazin-1-yl)-1,1-dioxo-2-pyridin-4-ylmethyl-1,4-dihydro-2H-1λ⁶-benzo[1,2,4]thiadiazin-3-one,
(43) 4-Benzyl-6-chloro-2-(5-methyl-furan-2-ylmethyl)-8-(4-methyl-piperazin-1-yl)-1,1-dioxo-1,4-dihydro-2H-1λ⁶-benzo[1,2,4]thiadiazin-3-one,
(44) 2,4-Dibenzyl-6-chloro-1,1-dioxo-8-piperazin-1-yl-1,4-dihydro-2H-1λ⁶-benzo[1,2,4]thiadiazin-3-one,
(45) 4-Benzyl-6-chloro-2-(2-fluoro-benzyl)-1,1-dioxo-8-piperazin-1-yl-1,4-dihydro-2H-1λ⁶-benzo[1,2,4]thiadiazin-3-one,
(46) 4-Benzyl-6-chloro-2-(3-fluoro-benzyl)-1,1-dioxo-8-piperazin-1-yl-1,4-dihydro-2H-1λ⁶-benzo[1,2,4]thiadiazin-3-one,
(47) 4-Benzyl-6-chloro-2-(2-chloro-benzyl)-1,1-dioxo-8-piperazin-1-yl-1,4-dihydro-2H-1λ⁶-benzo[1,2,4]thiadiazin-3-one,
(48) 4-Benzyl-2-(2-bromo-benzyl)-6-chloro-1,1-dioxo-8-piperazin-1-yl-1,4-dihydro-2H-1λ⁶-benzo[1,2,4]thiadiazin-3-one,
(49) 4-Benzyl-6-chloro-2-(4-iodo-benzyl)-1,1-dioxo-8-piperazin-1-yl-1,4-dihydro-2H-1λ⁶-benzo[1,2,4]thiadiazin-3-one,
(50) 4-Benzyl-6-chloro-2-(2-methyl-benzyl)-1,1-dioxo-8-piperazin-1-yl-1,4-dihydro-2H-1λ⁶-benzo[1,2,4]thiadiazin-3-one,
(51) 4-Benzyl-6-chloro-2-(2-methoxy-benzyl)-1,1-dioxo-8-piperazin-1-yl-1,4-dihydro-2H-1λ⁶ benzo[1,2,4]thiadiazin-3-one,
(52) 4-Benzyl-6-chloro-2-(3-methoxy-benzyl)-1,1-dioxo-8-piperazin-1-yl-1,4-dihydro-2H-1λ⁶-benzo[1,2,4]thiadiazin-3-one,
(53) 4-Benzyl-6-chloro-2-(4-methoxy-benzyl)-1,1-dioxo-8-piperazin-1-yl-1,4-dihydro-2H-1λ⁶-benzo[1,2,4]thiadiazin-3-one,
(54) 4-Benzyl-6-chloro-2-(3-hydroxy-benzyl)-1,1-dioxo-8-piperazin-1-yl-1,4-dihydro-2H-1λ⁶-benzol [1,2,4]thiadiazin-3-one,
(55) 4-Benzyl-6-chloro-2-(2-nitro-benzyl)-1,1-dioxo-8-piperazin-1-yl-1,4-dihydro-2H-1λ⁶-benzo[1,2,4]thiadiazin-3-one,
(56) 4-(4-Benzyl-6-chloro-1,1,3-trioxo-8-piperazin-1-yl-3,4-dihydro-1H-1λ⁶-benzo[1,2,4]thiadiazin-2-ylmethyl)-benzoic acid methyl ester,
(57) 4-(4-Benzyl-6-chloro-1,1,3-trioxo-8-piperazin-1-yl-3,4-dihydro-1H-1λ⁶-benzo[1,2,4]thiadiazin-2-ylmethyl)-benzonitrile,
(58) 4-Benzyl-6-chloro-1,1-dioxo-2-[(R)-1-phenyl-ethyl]-8-piperazin-1-yl-1,4-dihydro-2H-1λ⁶-benzo[1,2,4]thiadiazin-3-one,
(59) 4-Benzyl-6-chloro-1,1-dioxo-2-[(S)-1-phenyl-ethyl]-8-piperazin-1-yl-1,4-dihydro-2H-1λ⁶-benzo[1,2,4]thiadiazin-3-one,
(60) 4-Benzyl-6-chloro-1,1-dioxo-2-phenyl-8-piperazin-1-yl-1,4-dihydro-2H-1λ⁶-benzo[1,2,4]thiadiazin-3-one,
(61) 4-Benzyl-6-chloro-2-(2-methoxy-phenyl)-1,1-dioxo-8-piperazin-1-yl-1,4-dihydro-2H-1λ⁶-benzo[1,2,4]thiadiazin-3-one,
(62) 4-Benzyl-6-chloro-2-(3-methoxy-phenyl)-1,1-dioxo-8-piperazin-1-yl-1,4-dihydro-2H-1λ⁶-benzo[1,2,4]thiadiazin-3-one,
(63) 4-Benzyl-6-chloro-2-(4-methoxy-phenyl)-1,1-dioxo-8-piperazin-1-yl-1,4-dihydro-2H-1λ⁶-benzo[1,2,4]thiadiazin-3-one,
(64) 4-Benzyl-6-chloro-2-ethyl-1,1-dioxo-8-piperazin-1-yl-1,4-dihydro-2H-1λ⁶-benzo[1,2,4]thiadiazin-3-one,
(65) 4-Benzyl-6-chloro-2-propyl-1,1-dioxo-8-piperazin-1-yl-1,4-dihydro-2H-1λ⁶-benzo[1,2,4]thiadiazin-3-one,
(66) 4-Benzyl-6-chloro-2-[3-(3,5-dimethyl-phenyl)-propyl]-1,1-dioxo-8-piperazin-1-yl-1,4-dihydro-2H-1λ⁶-benzo[1,2,4]thiadiazin-3-one,
(67) 4-Benzyl-6-chloro-2-decyl-1,1-dioxo-8-piperazin-1-yl-1,4-dihydro-2H-1λ⁶-benzo[1,2,4]thiadiazin-3-one,
(68) 4-Benzyl-6-chloro-2-naphthalen-1-ylmethyl-1,1-dioxo-8-piperazin-1-yl-1,4-dihydro-2H-1λ⁶-benzo[1,2,4]thiadiazin-3-one,
(69) 4-Benzyl-6-chloro-2-(2-chloro-benzyl)-8-morpholin-4-yl-1,1-dioxo-1,4-dihydro-2H-1λ⁶-benzo[1,2,4]thiadiazin-3-one,
(70) 4-Benzyl-6-chloro-2-(2-chloro-benzyl)-1,1-dioxo-8-(4-pyridin-2-yl-piperazin-1-yl)-1,4-dihydro-2H-1λ⁶-benzo[1,2,4]thiadiazin-3-one,
(71) 4-Benzyl-6-chloro-2-(2-chloro-benzyl)-8-[(R)-3-methyl-piperazin-1-yl]-1,1-dioxo-1,4-dihydro-2H-1λ⁶-benzo[1,2,4]thiadiazin-3-one,
(72) 4-Benzyl-6-chloro-2-(2-chloro-benzyl)-8-[(S)-3-methyl-piperazin-1-yl]-1,1-dioxo-1,4-dihydro-2H-1λ⁶-benzo[1,2,4]thiadiazin-3-one,
(73) 4-Benzyl-6-chloro-2-(2-chloro-benzyl)-8-[(6S)-1,4-diazabicyclo[4.3.0]non-4-yl]-1,1-dioxo-1,4-dihydro-2H-1λ⁶-benzo[1,2,4]thiadiazin-3-one,
(74) 4-Benzyl-6-chloro-2-(2-chloro-benzyl)-8-[(6R)-1,4-diazabicyclo[4.3.0]non-4-yl]-1,1-dioxo-1,4-dihydro-2H-1λ⁶-benzo[1,2,4]thiadiazin-3-one, and
(75) 4-Benzyl-6-chloro-2-(2-chloro-benzyl)-8-[(6R)-1,4-diazabicyclo[4.4.0]dec-4-yl]-1,1-dioxo-1,4-dihydro-2H-1λ⁶-benzo[1,2,4]thiadiazin-3-one.

5. A method for preparing the compound of substituted-1,1-dioxo-benzo[1,2,4]thiadiazin-3-one ones of claim 1 as following Scheme 1, comprising the steps of:
(a) preparing an intermediate I by reaction of the compounds 2 and an amine in the presence of a base;
(b) preparing an intermediate II by cyclization of the compound of the intermediate I;
(c) preparing an intermediate III by substitution reaction on intermediate II in the presence of a base; and
(d) preparing a compound of Formula I by nucleophilic substitution reaction of the intermediate III using an amine. 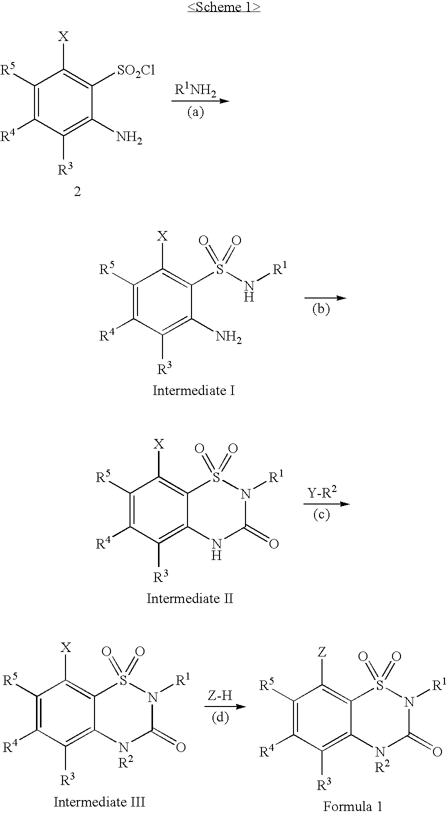
(wherein,
R¹˜R⁵ and Z are same as the aforementioned definition in claim 1;
X is a fluorine, chlorine, bromine, iodine or trifluoroacetate; and
Y is chlorine, bromine, iodine, methanesulfonate or p-toluenesulfonate).

6-8. (canceled)

9. The method according to claim 5, wherein cyclization of the step (b) is conducted by di- or tri-phosgen.

10. A pharmaceutical composition containing a compound of claim 1, or a pharmaceutically acceptable salt thereof.

11-12. (canceled)

13. A method for treating central nervous system disorders in a subject, comprising administering a pharmaceutically effective amount of the compound of claim 1 or a pharmaceutically acceptable salt thereof to the subject in need thereof.

14. The method according to claim 13, wherein the central nervous system disorder is selected from the group consisting of cognitive disorders, Alzheimers disease, anxiety, depression, schizophrenia, stress disorder, panic disorder, phobic disorder, obsessive compulsive disorder, post traumatic stress disorder, immune system depression, psychosis, paraphrenia, mania, convulsive disorder, personality disorders, migraine, drug addiction, alcoholism, obesity, eating disorders, and sleep disorders.

15. The method according to claim 13, wherein the central nervous system disorder is 5-HT6 receptor mediated central nervous system disorder.

16. A method for treating 5-HT6 receptor mediated disorders in a subject, comprising administering a pharmaceutically effective amount of the compound of claim 1 or a pharmaceutically acceptable salt thereof to the subject in need thereof.


SEQUENCE LISTING (2 sequences)

SEQ ID NO: 1 - DNA, 24 bp, Artificial Sequence
  forward primer for serotonin 5HT receptor
tcatctgctttcccgccaccctat

SEQ ID NO: 2 - DNA, 24 bp, Artificial Sequence
  reverse primer for serotonin 5HT receptor
tcagggtctgggttctgctcaatc